(19) United States
(12) Patent Application Publication
(10) Pub. No.: US 2013/0046171 A1
(43) Pub. Date: Feb. 21, 2013

(54) DEVICE AND METHOD FOR ACCESSING AND TREATING DUCTS OF MAMMARY GLANDS

(71) Applicants: Jerald A. Johansen, Aliso Viejo, CA (US); Roman Slizynski, Foothill Ranch, CA (US); David Licata, Los Angeles, CA (US); Troy M. Bremer, Dana Point, CA (US); Susan M. Love, Pacific Palisades, CA (US)

(72) Inventors: Jerald A. Johansen, Aliso Viejo, CA (US); Roman Slizynski, Foothill Ranch, CA (US); David Licata, Los Angeles, CA (US); Troy M. Bremer, Dana Point, CA (US); Susan M. Love, Pacific Palisades, CA (US)

(21) Appl. No.: 13/456,782

(22) Filed: Apr. 26, 2012

Related U.S. Application Data
(63) Continuation of application No. 12/424,506, filed on Apr. 15, 2009, now abandoned.
(60) Provisional application No. 61/145,946, filed on Jan. 20, 2009.
(60) Provisional application No. 61/045,241, filed on Apr. 15, 2008.

(51) Int. Cl. A61M 25/00; A61B 6/03; A61B 6/02; A61B 6/00; A61B 5/055

(52) U.S. Cl. 600/420; 600/434; 604/264; 604/500

(57) ABSTRACT

This application relates to a device for the local delivery of a substance to a mammary duct by ductal cannulation via an orifice on a nipple, wherein the substance may any substance or combinations of substances, such as for example, compositions capable of forming a solid or semisolid gel within the ducts, a marker, and/or an active agent which is preferably effective in treating and/or preventing breast cancer. More particularly, a device is disclosed comprising a probe for locating the orifice on the nipple, wherein the device is further configured to cannulate the duct and allow local delivery of a substance(s).

### RELATED APPLICATIONS

This application is a continuation of U.S. patent application Ser. No. 12/424,506, filed Apr. 15, 2009, which claims priority under 35 U.S.C. §119(e) to U.S. provisional application No. 61/045,241, filed Apr. 15, 2008 and provisional application No. 61/145,946 filed Jan. 20, 2009. The full disclosures of the prior applications of which are incorporated herein by reference in their entirety.

### BACKGROUND OF THE INVENTION

1. Field of the Invention

This application relates to a device for the local delivery of a substance to a mammary duct by ductal cannulation via an orifice on a nipple, wherein the substance may any substance or combinations of substances, such as for example, compositions capable of forming a solid or semisolid gel within the ducts, a marker, and/or an active agent which is preferably effective in treating and/or preventing breast cancer. More particularly, a device is disclosed comprising a probe for locating the orifice on the nipple, wherein the device is further configured to cannulate the duct and allow local delivery of a substance(s).

2. Description of the Related Art

Mammary glands are exocrine glands, which are glands that release a secretion external to or at the surface of an organ by means of a canal or duct. In addition to mammary glands, examples of exocrine glands include the prostate, liver, gall bladder, pancreas, kidneys, sweat glands, and salivary glands. Cancers of exocrine glands pose a major health problem, frequently resulting in death. Currently, cancers of the breast and prostate are among the leading causes of death among women and men, respectively.

The mature human breast comprises from six to nine major ducts, which emanate from the nipple, serially branch into ducts and terminate in lobuloalveolar structures (Russo et al., Lab. Invest. 62(3): 244-278 (1990)). This branching network of ducts is composed of epithelial cells in a supporting matrix of connective tissue and endothelial cells.

Analysis of ductal secretions from human breasts has been used to diagnose biological conditions of the breast ducts (1-4; see list of references hereinafter). Most of these studies pooled secretions or nipple aspirate fluid (NAF) from several ducts for the analysis. Thus, the secretions of an individual duct are not identified in these studies. More recently, breast duct access has been incorporated into ductal fluid analyses and study protocols (5-7). Various methods have been employed in order to identify the breast ducts for purposes including duct fluid analysis (8-15). Additionally, breast ducts have been accessed by ductscope (15, 16). Ductal cannulation is proposed for delivery of agents (WO 97/05898 and (20) incorporated herein by reference in their entirety). Ductography, or mammary duct contrast examination, involves cannulation and injection of a ductal orifice (17-19), a process that is generally painless and devoid of complications (20).

Ductal access is also required for performing lavage procedures on a milk duct to deliver an agent and/or to retrieve cells from the duct for analysis. The challenge for the procedure can be finding a duct, or multiple ducts, for access. In one conventional technique, the identification of ducts has been a separate process from accessing the ducts using a cannula or the like.

Tissues removed from the human female breast during surgery and autopsy have been examined in numerous studies directed to the nature and site of origin of neoplastic growth. Subgross sampling and histological confirmation have enabled pathological characterization of entire breasts, leading to the postulation of the existence of four major possible sites of origin of mammary carcinomas, namely ducts, terminal ducts, ductules, and acini (Russo et al., supra). Ductal origin is supported by the presence of more extensive epithelial proliferations, which are presumed to be preneoplastic, in surgically removed cancerous breasts as compared to nonmalignant breasts removed during autopsies (Russo et al., supra).

With a cumulative lifetime risk of a woman developing breast cancer estimated to be 1 in 9, there is an urgent need to develop therapeutic methods of treatment that are more effective, less invasive and accompanied by fewer side effects and prophylactic methods of treatment that are more effective than increased and intensified physical monitoring and less extreme than radical mastectomy. In spite of the recent discovery of the heritable breast cancer susceptibility loci, BRCA1 (Miki et al., Science 266:66-71 (1994)) and BRCA2, and other cancer susceptibility loci, and the increasing ability of physicians to identify women with elevated breast cancer risk, prophylactic methods are still currently limited to physical monitoring and prophylactic mastectomy.

### SUMMARY OF THE INVENTION

An apparatus is disclosed for intraductal delivery of an agent to a target duct, comprising a catheter having a distal tip and a lumen, the distal tip comprising a probe for locating a ductal orifice associated with the target duct, wherein the catheter is further sized and configured for cannulating the target duct via the orifice.

The probe may be operably coupled to a processing unit. The operable coupling may comprise a conductor or transmitter for relaying a signal from the probe to the processing unit. The processing unit may be configured to generate a user signal when the relayed signal is characteristic of a probe position over the ductal orifice. The user signal can be seen and/or heard by the user.

The agent may be toxic to at least some epithelial and/or non-epithelial cells associated with the duct. The agent may be a conventional chemotherapy drug. The agent may be selected from the group consisting of genistein, okadaic acid, 1-β-D-arabinofuranosyl-cytosine, arabinofuranosyl-5-aza-cytosine, cisplatin, carboplatin, actinomycin D, asparaginase, bis-chloro-ethyl-nitroso-urea, bleomycin, chlorambucil, cyclohexyl-chloro-ethyl-nitroso-urea, cytosine arabinoside, daunomycin, etoposide, hydroxyurea, melphalan, mercaptopurine, mitomycin C, nitrogen mustard, procarbazine, teniposide, thioguanine, thiotepa, vincristine, 5-fluorouracil, 5-fluorocytosine, adriamycin, cyclophosphamide, methotrexate, vinblastine, doxorubicin (and liposomal doxorubicin), leucovorin, taxol, anti-estrogen agents such as tamoxifen, intracellular antibodies against oncogenes, the flavonol quercetin, Guan-mu-tong extract, retinoids, nontoxic retinoid analogs, sclerosants, immunotherapeutics (such as HERCEPTIN™) and monoterpenes. The agent may further comprise a carrier. The agent may be a vector comprising a gene. The agent may be a cytolytic virus. The agent may comprise a cytokine or a hematopoietic growth factor.

In preferred variations to the apparatus, the distal tip may further comprise an atraumatic configuration. The apparatus may also comprise an infusion port for administering the agent, and wherein the distal tip is sealed.

A breast duct access device is disclosed in accordance with another aspect of the invention. The device comprises: a circuit apparatus configured to: complete an electrical circuit when placed in electrical contact with the breast and measure an electrical value on the circuit; and a duct accessing apparatus for accessing the breast duct when the electrical value is a characteristic value.

The circuit apparatus may comprise a test electrode, which may further comprise an atraumatic tip. The device may further comprise a reference electrode configured to engage against a body surface. The device may further comprise an electrical potential source in electrical communication with the reference electrode and the test electrode.

The duct accessing apparatus of the device may comprise a catheter. The duct accessing apparatus may further comprise a guidewire. The guidewire may maintain a constant position relative to the catheter. The guidewire may be moveable relative to the catheter. The guidewire may be affixed to a hub, the hub being detachably engageable with the catheter.

The device may also comprise a marker tube having an inner diameter sized to accommodate the catheter therein and an outer diameter sized to be accommodated within an orifice of the duct.

The duct accessing apparatus may further comprise a source for an agent, the source configured to provide the agent to the duct through the duct accessing apparatus.

A kit is disclosed, comprising: an analyzer configured to indicate a characteristic electrical value in an electrical circuit comprising a duct of a mammary gland; a catheter operable to access the duct when the analyzer indicates the characteristic electrical value; and a therapeutically or prophylactically effective amount of an agent in a form capable of introduction through the catheter.

Another kit is disclosed for destroying mammary gland ductal epithelium, comprising: a duct accessing device comprising: a pair of electrodes, at least one of which is configured to establish electrical communication with a nipple surface; and a catheter sized to access the mammary gland; and an agent capable of introduction through the catheter and effective to destroy at least a portion of the ductal epithelium.

Another kit is disclosed, comprising: a nontoxic marker dye that indicates the location of ductal orifices when injected proximate to the ducts of the breast; a catheter operable to access the indicated ductal orifices; and a therapeutic agent capable of introduction through the catheter.

A method of treating or preventing breast cancer is disclosed, comprising: providing a catheter having an atraumatic tip and a lumen; locating multiple ductal orifices on a nipple using at least one of a duct locating instrument, a magnifying instrument, and a compound that visually enhances the location of the ductal orifices; cannulating at least a first ductal orifice by inserting the atraumatic tip through the first orifice and into a first milk duct; and delivering an agent through the lumen and into the first milk duct. The agent preferably destroys at least some epithelial cells exposed thereto.

The method may further comprise: removing the atraumatic tip from the first orifice; cannulating at least a second ductal orifice by inserting the atraumatic tip through the second orifice and into a second milk duct; and delivering an agent through the lumen and into the second milk duct.

The method may further comprise: providing a second catheter having an atraumatic tip and a lumen; cannulating at least a second ductal orifice by inserting the atraumatic tip of the second catheter into the second orifice; and delivering an agent through the lumen of the second catheter and into the second orifice. The duct locating instrument may comprise the atraumatic tip.

A method is disclosed for cannulating a duct of a mammary gland via a ductal orifice, comprising: providing an apparatus for accessing ductal orifices, the apparatus comprising a catheter having a distal tip and a lumen, the distal tip comprising a duct probe for identifying the presence of a ductal orifice; and cannulating at least a first ductal orifice by inserting the distal tip into the first orifice.

The method may further comprise: removing the distal tip from the first orifice; and cannulating at least a second ductal orifice by inserting the distal tip into the second orifice. The provided apparatus may further comprise a second catheter having a distal tip and a lumen, and further comprising: cannulating at least a second ductal orifice by inserting the distal tip of the second catheter into the second orifice. The method may further comprise: delivering an agent through the lumen and into the first orifice.

A method is disclosed for treating a mammary gland in need thereof, comprising: passing an electrical current or potential through the mammary gland and adjacent tissue; identifying at least one ductal orifice of the mammary gland based on measured values of the electrical current or potential; introducing a catheter having a distal tip and a lumen into at least a first ductal orifice; and administering, via the catheter, an agent which treats the mammary gland.

The method may further comprise: removing the catheter from the first orifice; cannulating at least a second ductal orifice by inserting the distal tip into the second orifice; and delivering an agent that destroys epithelial cells through the lumen and into the second orifice.

The method may further comprise: providing a second catheter having a distal tip and a lumen; cannulating at least a second ductal orifice by inserting the distal tip of the second catheter into the second orifice; and delivering an agent that destroys epithelial cells through the lumen and into the second orifice.

A method of distinguishing types of biological tissue is also disclosed. The method comprises: determining electrical characteristics of the biological tissue at least at a first frequency and at a second frequency, wherein the second frequency is different than the first frequency; calculating one or more relative relationships between the electrical characteristic at the first frequency and the electrical characteristic at the second frequency; and determining whether the biological tissue corresponds to a particular type of biological tissue at least partially based on the one or more relative relationships.

The method may further comprise comparing the one or more relative relationships to selected criteria, and determining whether the biological tissue corresponds to the particular type of biological tissue at least partially based on the comparison. The selected criteria may correspond to a multi-dimensional space. In one embodiment, at least a portion of the selected criteria is retrieved from a lookup table.

The step of calculating one or more relative relationships may comprise calculating at least one of a magnitude ratio, a phase ratio, or a phase difference.

The step of determining electrical characteristics of the biological tissue may comprise determining both a real component and an imaginary component of an admittance or an impedance of the biological tissue for both the first frequency and the second frequency. Alternatively, the step of determining electrical characteristics may comprise applying a voltage signal, detecting a corresponding current, and determining a magnitude and phase relationship between the voltage signal and the detected current. Alternatively, the step of determining electrical characteristics may comprise applying a current signal, detecting a corresponding voltage, and determining a magnitude and phase relationship between the current signal and the detected voltage.

In one embodiment, the method may further comprise repeatedly alternating between the first frequency and the second frequency.

In one embodiment, the method may further comprise generating a first sine wave having the first frequency and a second sine wave having the second frequency.

In one embodiment, the method may further comprise generating a coherent signal having both the first frequency and the second signal, wherein the signal is coherent.

In one embodiment of the method, the second frequency is at least twice the first frequency. In another embodiment, the second frequency is a multiple of the first frequency and less than about 1 MHz. In another embodiment, the second frequency is about 4 times the first frequency.

In one embodiment, the method may further comprise activating at least one of an audible, visual, or vibrating indication at least partly in response to the determination.

In one embodiment, the particular type of biological tissue corresponds to a milk duct of a human breast.

An apparatus for distinguishing types of biological tissue is disclosed. The apparatus comprises: a circuit analyzer configured to determine electrical characteristics of the biological tissue at least at a first frequency and at a second frequency, wherein the second frequency is higher than the first frequency; a processing circuit configured to calculate one or more relative relationships between the electrical characteristic at the first frequency and the electrical characteristic at the second frequency and to determine whether the biological tissue corresponds to a particular type of biological tissue at least partially based on the one or more relative relationships.

The processing circuit may be further configured to compare the one or more relative relationships to selected criteria, and to determine whether the biological tissue corresponds to the particular type of biological tissue at least partially based on the comparison. The selected criteria preferably correspond to a multi-dimensional space.

In one embodiment, the apparatus may further comprise a lookup table, wherein the processing circuit is configured to retrieve at least a portion of the selected criteria from a lookup table. The processing circuit may be configured to calculate at least one of a magnitude ratio, a phase ratio, or a phase difference. Alternatively, the processing circuit may be configured to calculate a magnitude ratio, a phase ratio, and a phase difference.

The circuit analyzer may be configured to determine both a real component and an imaginary component of an admittance or an impedance of the biological tissue for both the first frequency and the second frequency. Alternatively, the circuit analyzer may be configured to generate a voltage signal, to detect a corresponding current, and to determine a magnitude and phase relationship between the voltage signal and the detected current. Alternatively, the circuit analyzer may be configured to apply a current signal, to detect a corresponding voltage, and to determine a magnitude and phase relationship between the current signal and the detected voltage. In one embodiment, the circuit analyzer is configured to repeatedly alternate between the first frequency and the second frequency. In one embodiment, the circuit analyzer is configured to generate a first sine wave having the first frequency and a second sine wave having the second frequency. In one embodiment, the circuit analyzer is configured to generate a coherent signal having both the first frequency and the second signal. The second frequency may be at least twice the first frequency. Alternatively, the second frequency is a multiple of the first frequency.

The apparatus preferably also comprises an audible, visual, or vibrating indicator, wherein the processing circuit is configured to activate the indicator at least partly in response to the determination.

The particular type of biological tissue may correspond to a milk duct of a human breast.

A method is disclosed for treating a mammary gland by delivering an agent within a hydrogel, comprising: cannulating a ductal orifice by inserting a catheter through the ductal orifice and into a milk duct; and delivering a liquid composition comprising an agent through the catheter and into the milk duct, wherein the liquid composition gels within the milk duct to form a hydrogel.

In another embodiment, a device and methods are disclosed for the local delivery of a gel composition to a mammary duct by ductal cannulation via an orifice on a nipple, wherein the gel composition is effective in augmenting breast aesthetics.

A kit is disclosed in accordance with an embodiment of the invention, comprising: an analyzer configured to indicate a characteristic electrical value in an electrical circuit comprising a duct of a mammary gland; a catheter operable to access the duct when the analyzer indicates the characteristic electrical value; and a gel composition in an amount effective to augment breast size which is capable of introduction through the catheter.

A method of breast augmentation is disclosed, comprising: cannulating a ductal orifice by inserting a catheter through the ductal orifice and into a milk duct; and delivering a liquid composition through the catheter and into the milk duct, wherein the liquid composition gels within the milk duct to form a hydrogel.

A method of aesthetically enhancing a mammary gland in need thereof is disclosed, comprising: passing an electrical current or potential through the mammary gland and adjacent tissue; identifying a ductal orifice of a milk duct of the mammary gland based on measured values of the electrical current or potential; introducing a catheter having an atraumatic distal tip and a lumen into the ductal orifice; and administering an effective amount of a gel composition into the milk duct through the catheter.

A method of breast augmentation is disclosed, the method comprising: determining electrical characteristics of a region of breast tissue at least at a first frequency and at a second frequency, wherein the second frequency is different than the first frequency; calculating one or more relative relationships between the electrical characteristic at the first frequency and the electrical characteristic at the second frequency; locating the region of breast tissue corresponding to a ductal orifice at least partially based on the one or more relative relationships; and infusing a gel composition through the ductal orifice.

In a first aspect, a kit is provided, the kit comprising an analyzer configured to indicate a characteristic electrical value in an electrical circuit comprising a duct of a mammary gland, a catheter operable to access the duct when the analyzer indicates the characteristic electrical value, wherein the catheter comprises an electrode functionally connected to the analyzer; and a therapeutically or prophylactically effective amount of an agent in a form capable of introduction through the catheter.

In an embodiment of the first aspect, the agent is effective to destroy at least a portion of the ductal epithelium.

In a second aspect, a method of delivering material to a mammary gland is provided, the method comprising passing an electrical current or potential through the mammary gland and adjacent tissue with an electrode, identifying at least one ductal orifice of the mammary gland based on determination of an electrical parameter determined by passing the electrical current or potential, wherein the determination is made by evaluating the characteristics of a resistance-inductance or a resistance-inductance-capacitance circuit, introducing a catheter having a distal tip and a lumen into at least a first ductal orifice, wherein the cannula acts as the electrode, and administering, via the catheter, a material to the mammary gland.

In an embodiment of the second aspect, the material comprises at least one of a hydrogel, a nanocarrier, a nanogel particle, an aggregated nanogel particle, an imaging agent, and a therapeutic or prophylactic agent.

In an embodiment of the second aspect, the material comprises a crosslinked PEG.

In an embodiment of the second aspect, the material comprises an imaging agent associated with a hydrogel or a nanocarrier or a nanogel particle or an aggregated nanogel particle.

In an embodiment of the second aspect, the material comprises an imaging agent covalently bonded to a hydrogel or a nanocarrier or a nanogel particle or an aggregated nanogel particle.

In an embodiment of the second aspect, the material comprises a dye or contrasting agent.

In an embodiment of the second aspect, the material is an imaging agent that is visible by visual observation or by radiographic, MRI, PET, or SPEC examination.

In an embodiment of the second aspect, the material is an epithelial cell destroying material.

In an embodiment of the second aspect, the material is a chemotherapeutic material.

In an embodiment of the second aspect, the material is selected from the group consisting of genistein, okadaic acid, 1-β-D-arabinofuranosyl-cytosine, arabinofuranosyl-5-aza-cytosine, cisplatin, carboplatin, actinomycin D, asparaginase, bis-chloro-ethyl-nitroso-urea, bleomycin, chlorambucil, cyclohexyl-chloro-ethyl-nitroso-urea, cytosine arabinoside, daunomycin, etoposide, hydroxyurea, melphalan, mercaptopurine, mitomycin C, nitrogen mustard, procarbazine, teniposide, thioguanine, thiotepa, vincristine, 5-fluorouracil, 5-fluorocytosine, adriamycin, cyclophosphamide, methotrexate, vinblastine, doxorubicin (and liposomal doxorubicin), leucovorin, taxol, anti-estrogen agents such as tamoxifen, intracellular antibodies against oncogenes, the flavonol quercetin, Guan-mu-tong extract, retinoids, nontoxic retinoid analogs, sclerosants, immunotherapeutics, HERCEPTIN™, and monoterpenes.

In an embodiment of the second aspect, the material forms a map of a ductal network.

In an embodiment of the second aspect, the material forms a map of a ductal network and at least a portion of the ductal network mapped is surgically removed.

In a third aspect, a method of distinguishing types of biological tissue is provided, the method comprising determining electrical characteristics of the biological tissue at least at a first frequency and at a second frequency, wherein the second frequency is different than the first frequency, utilizing a catheter which comprises an electrode; calculating one or more relative relationships between the electrical characteristic at the first frequency and the electrical characteristic at the second frequency, said relationships comprising at least one of calculating a magnitude ratio, a phase ratio, and a phase difference; and determining whether the biological tissue corresponds to a particular type of biological tissue at least partially based on the one or more relative relationships.

In an embodiment of the third aspect, the method further comprises comparing the one or more relative relationships to selected criteria, and determining whether the biological tissue corresponds to the particular type of biological tissue at least partially based on the comparison.

In an embodiment of the third aspect, the method further comprises comparing the one or more relative relationships to selected criteria, and determining whether the biological tissue corresponds to the particular type of biological tissue at least partially based on the comparison, wherein the selected criteria corresponds to a multi-dimensional space.

In an embodiment of the third aspect, the method further comprises comparing the one or more relative relationships to selected criteria, and determining whether the biological tissue corresponds to the particular type of biological tissue at least partially based on the comparison, wherein at least a portion of the selected criteria is retrieved from a lookup table.

In an embodiment of the third aspect, the method further comprises activating at least one of an audible, visual, or vibrating indication at least partly in response to the determination.

In an embodiment of the third aspect, the particular type of biological tissue corresponds to a milk duct of a human or animal breast.

In a fourth aspect, an apparatus for distinguishing types of biological tissue is provided, the apparatus comprising: a circuit analyzer configured to determine electrical characteristics of the biological tissue at least at a first frequency and at a second frequency, wherein the second frequency is higher than the first frequency; a catheter which comprises an electrode; a processing circuit configured to calculate one or more relative relationships between the electrical characteristic at the first frequency and the electrical characteristic at the second frequency and to determine whether the biological tissue corresponds to a particular type of biological tissue at least partially based on the one or more relative relationships, wherein determining characteristics comprises calculating at least one of a magnitude ratio, a phase ratio, or a phase difference.

In an embodiment of the fourth aspect, the processing circuit is further configured to compare the one or more relative relationships to selected criteria, and to determine whether the biological tissue corresponds to the particular type of biological tissue at least partially based on the comparison.

In an embodiment of the fourth aspect, the processing circuit is further configured to compare the one or more relative relationships to selected criteria, and to determine whether the biological tissue corresponds to the particular type of biological tissue at least partially based on the comparison, and the selected criteria corresponds to a multi-dimensional space.

In an embodiment of the fourth aspect, the processing circuit is further configured to compare the one or more relative relationships to selected criteria, and to determine whether the biological tissue corresponds to the particular type of biological tissue at least partially based on the comparison, and the apparatus further comprises a lookup table, wherein the processing circuit is configured to retrieve at least a portion of the selected criteria from a lookup table.

In an embodiment of the fourth aspect, the circuit analyzer is configured to repeatedly alternate between the first frequency and the second frequency.

In an embodiment of the fourth aspect, the circuit analyzer is configured to generate a first sine wave having the first frequency and a second sine wave having the second frequency.

In an embodiment of the fourth aspect, the apparatus further comprises an audible, visual, or vibrating indicator, wherein the processing circuit is configured to activate the indicator at least partly in response to the determination.

In an embodiment of the fourth aspect, the particular type of biological tissue corresponds to a milk duct of a human or animal breast.

In a fifth aspect, a method of treating a mammary gland by delivering an agent within a hydrogel is provided, the method comprising: identifying a breast duct through evaluation of electrical signals, cannulating a ductal orifice by inserting a catheter through the ductal orifice and into a milk duct, wherein the catheter comprises an electrode used in identifying the breast duct; and delivering an agent through the catheter.

### BRIEF DESCRIPTION OF THE DRAWINGS

FIG. 1 shows a schematic view of a breast duct accessing apparatus in accordance with an embodiment of the invention, shown in situ on a nipple of a breast.

FIG. 2 shows a schematic cutaway view of an embodiment of a catheter structure in which a probe electrode is mounted on a guidewire that is accommodated within the lumen of the catheter structure.

FIGS. 3A-3C show a side view of an embodiment of a catheter and electrode in which a guidewire is attached via a glue joint to a threaded hub.

FIG. 4 is a side view of an embodiment of an apparatus in which a flexible metal hypotube functions as a catheter and electrode.

FIG. 5 is a side view of an embodiment in which a metal hypotube is provided at the distal end of an ultraflexible catheter.

FIG. 6 is a side view of an embodiment in which a coil is provided within the distal end of a catheter so as to protrude beyond the distal end.

FIG. 7A is a side view of an embodiment in which a marker tube is advanced over a standard catheter body.

FIGS. 7B-7D show the stages of advancement of the marker tube into the ductal orifice.

FIG. 8A illustrates an embodiment in which the distal end of the catheter is sealed and a lateral infusion port is provided near the distal end of the catheter.

FIG. 8B illustrates an embodiment in which the physical properties of the catheter, such as wall thickness or outer diameter, change from the proximal end to the distal end thereof.

FIG. 8C illustrates an embodiment in which a hypotube having an atraumatic ball formed thereon is accommodated within the distal end of the catheter.

FIG. 8D illustrates an embodiment of a structure employed at the proximal end of the catheter.

FIG. 9 illustrates an equivalent circuit for tissue, such as tissue including a milk duct.

FIG. 10 is a chart illustrating magnitude ratios of admittance for fixed conductances, a 4:1 frequency ratio, and a range of capacitance values.

FIG. 11 is a chart illustrating phase ratios for admittance for fixed conductances, a 4:1 frequency ratio, and a range of capacitance values.

FIG. 12 is a chart illustrating phase differences for admittance for fixed conductances, a 4:1 frequency ratio, and a range of capacitance values.

FIG. 13 is a 3-dimensional chart (x, y, z) of actual test data taken from human breast tissue removed via mastectomy.

FIGS. 14 and 15 illustrate two views of the same example of using a 2-dimensional threshold to distinguish different types of tissue.

FIG. 16 is a block diagram of an analyzer according to an embodiment of the invention.

FIG. 17 illustrates a method for distinguish types of tissue based on electrical characteristics.

FIG. 18 depicts a cable assembly of a test device.

FIG. 19 depicts an extension cable of a test device.

FIG. 20 depicts an electronic sensing unit of a test device.

### DETAILED DESCRIPTION OF THE PREFERRED EMBODIMENTS

The present disclosure relates to methods, devices, and kits for locating individual ductal orifices on a nipple surface of a mammalian breast, typically a human female breast, for accessing the mammary or milk ducts once the ductal orifices have been located, and, in certain preferred embodiments, for administering therapeutic and/or prophylactic agents locally to the ducts via direct cannulation of the located orifice and associated duct.

Embodiments of the device involve the use of a catheter having a distal tip and a lumen, wherein the distal tip comprises a probe for precisely locating the ductal orifice. The probe can include a conductive tip surface employed as an electrode in order to detect characteristic impedance or capacitance values indicative of the presence of a ductal orifice as described below. In further embodiments, the conductive tip surface can also be atraumatic in order to avoid tissue damage when employing the probe for locating and subsequently cannulating the ductal orifice. When a flexible metal hypotube is employed as part of the catheter, a solder ball or a coil at the distal tip may be used as the conductive tip surface. Alternatively, when a polymer body is employed as part of the catheter, a short length of metal tubing or wire can be inserted in the distal end of the catheter body, and a solder ball or a coil can be attached at the distal tip thereof or the wire or tube can be melted to form a ball to provide the conductive tip surface.

In further embodiments, the probe may comprise a conductive structure situated at the distal end of a guidewire that is passed through the catheter. The structure can include, for example, a coil or a solder ball. Any other suitable conductive structure of a size to allow it to pass through the catheter along with the guidewire can also be employed. Alternatively, the end of the guidewire itself can be treated so as to render it atraumatic, for example by a slight rounding or flattening of the distal end of the wire or a melting of the distal end to create a ball shape.

In other embodiments, a marker tube structure can also be provided. Such marker tubes are sized so that at least a distal portion of the catheter, including e.g., the probe, can be accommodated within the tube, and the tube itself can be inserted into a ductal orifice after it is detected and located. The inserted marker tube serves to mark the location of the duct for later access. The marker tube can be relatively rigid, so as to remain open despite the pressure exerted by the surrounding ductal tissue, or can be very thin walled so as to collapse on removal of the device. In a preferred embodiment, at least one marker tube is provided on the outside of the probe, and when a ductal orifice is detected using the probe, the marker tube is advanced over the probe and inserted into the duct.

The duct probe described above may be connected to a processing unit by a conductor that conducts signals from the probe to a processing unit in order to detect the presence of a ductal orifice at the distal tip. The conductor may comprise a catheter body, when that body is made of a conductive material such as metal. Alternatively, the conductor may comprise a metal wire such as a guidewire. The signal transmitted from the duct probe to the processing unit is a characteristic electrical signal, such as an impedance value, a conductance value, or a reactance value.

The processing unit may comprise an indicator that displays the value of the characteristic electrical signal. The signal value itself may be indicated. Alternatively, the processing unit may determine whether the probe is currently located at a ductal orifice based on the characteristic electrical signal, and may indicate through an output module (e.g., an LCD display and/or speaker—or any other visual and audio output devices known in the art) the results of that determination through a visual and/or audible signal. The determination of whether the probe is currently located at a ductal orifice may be conducted by comparing the characteristic electrical signal or computations performed on the signals to preset reference values. Alternatively, the reference value may be one generated by placement of the probe on the breast where a ductal orifice is known not to be present. Alternatively, the reference value may be generated by placement of a separate reference electrode at one or more locations on the breast where a ductal orifice is known not to be present.

The outer diameter of the catheter is preferably within a range of 0.008-0.040 inches. More preferably, the outer diameter is within a range of 0.010-0.032 inches. Most preferably, the outer diameter is within a range of 0.012-0.030 inches. Furthermore, a catheter that is designed to be grasped at its proximal end with or without the aid of a grasping tool preferably has a length within a range of 2-20 inches. More preferably, the length is within a range of 4-18 inches. When the catheter is designed to be grasped by an ergonomic hub it preferably has a length within a range of 0.120-1.250 inches. More preferably, the length is within a range of 0.250-1.000 inches. Most preferably, the length is within a range of 0.375-0.750 inches.

In embodiments in which a hydrogel is to be delivered, the apparatus further comprises one or more reservoirs for a gel composition(s). The reservoir is preferably in fluid communication with the lumen of the catheter to facilitate administration of the gel composition to the duct once the catheter has been inserted into the orifice. The reservoir is also preferably coupled to a pressurization source to facilitate delivery of the gel composition through the lumen and into the cannulated duct. For example, the reservoir may be a conventional syringe and the pressurization source may be the syringe piston. Alternatively, the reservoir may comprise a peristaltic mechanism or gravity feed mechanism to provide pressure. In another variation, the pressurization source may be pressurized gas in communication with the reservoir. Of course, any other structural means known in the art for causing the gel composition in the reservoir to exit the catheter lumen and enter the duct may be employed.

In embodiments in which an agent used for the prophylaxis or treatment of breast cancer is administered to a duct after the ductal orifice is located, the apparatus may further comprise a reservoir for the agent. The reservoir is preferably in fluid communication with the lumen of the catheter to facilitate administration of the agent to the duct once the catheter has been inserted into the orifice. The reservoir is also preferably coupled to a pressurization source to facilitate delivery of the agent through the lumen and into the cannulated duct. For example, the reservoir may be a conventional syringe and the pressurization source may be the syringe piston. Alternatively, the reservoir may comprise a peristaltic mechanism or gravity feed mechanism to provide pressure. In another variation, the pressurization source may be pressurized gas in communication with the reservoir. Of course, any other structural means known in the art for causing the agent in the reservoir to exit the catheter lumen and enter the duct may be employed.

In a further embodiment, a device for accessing a breast duct is provided that includes a circuit apparatus configured to determine an electrical signal characteristic of a portion of a breast duct. The circuit apparatus may comprise one or more of a test electrode that may comprise a conductive atraumatic tip, a reference electrode configured to engage against a body surface, or an electrical potential source in electrical communication with the reference electrode and the test electrode. In further embodiments, the device may comprise a catheter or a guidewire or a combination of catheter and guidewire. The guidewire may maintain a constant position with respect to the catheter or be moveable relative to the catheter. The conductive tip of the catheter or guidewire or combination guidewire catheter may have an atraumatic tip. The device may also comprise a marker tube such as those described above, having an inner diameter sized to accommodate the catheter therein and an outer diameter sized to be accommodated within a duct. In another embodiment, the device may also be provided with a source for an agent that is configured to provide the agent to the duct through the duct accessing apparatus.

In another embodiment, an apparatus for providing an agent to a breast duct is provided that comprises a catheter having a distal tip and a lumen, the distal tip comprising a duct probe for identifying the presence of a ductal orifice, and a reservoir for the agent, the reservoir preferably being reversibly coupled to and in fluid communication with the lumen. The catheter may comprise any material known in the art for such devices, including polymers and metals such as stainless steel or nitinol. The duct probe may comprise a separate structure at the distal tip of the catheter, such as a solder ball or coil, or may be a part of the catheter itself, particularly where the catheter comprises a conductive material. In a further embodiment, the duct probe may be connected to a processing unit by a conductor that conducts signals from the probe to the processing unit, the processing unit comprising an indicator for a characteristic electrical signal. In certain embodiments, the conductor may be a wire such as a guidewire.

The characteristic electrical signal may be an impedance value, a capacitance value, a reactance value, or any other electrical value that can be employed to distinguish a ductal orifice from the surrounding tissue. In an embodiment, the duct probe transmits the characteristic electrical signal when the duct probe is located at a potential ductal orifice.

In certain embodiments, the agent may be effective to treat or prevent breast cancer; it may comprise an epithelium-destroying agent, a cytotoxic agent, a vector comprising a gene, a cytolytic virus, a cytokine or hematopoietic growth factor, or any other agent that may be employed to destroy or render quiescent breast duct epithelial cells or treat or prevent breast cancer. Because of the differential permeability of the epithelial lining of the duct with respect to the molecular size, charge and permeability characteristics of the particular agent(s) and/or carriers thereof, the agent(s) may not only contact the ductal epithelium, but may also traverse the epithelium and contact underlying stromal and other non-epithelial cells. Indeed, in some embodiments, the agent(s) may be modified and or associated (covalently or non-covalently) with other molecules in order to target delivery to selected cells.

A kit for treating or preventing cancer in a mammary gland is disclosed in accordance with certain embodiments. The kit may comprise an analyzer configured to indicate a characteristic electrical value in an electrical circuit comprising a duct of the mammary gland; a catheter operable to access the duct when the analyzer indicates the characteristic electrical value; and a therapeutic agent capable of introduction through the catheter and effective to treat or prevent the cancer. The analyzer may comprise one or more of a processing unit, a conductor, and an indicator for indicating when the characteristic electrical value has been provided. The catheter may comprise a distal duct probe for detecting the characteristic electrical value. In further embodiments, the catheter may be sized so as to be accommodated within a marker tube, which itself is sized to access the duct. In further embodiments, the kit may also comprise a guidewire, which may be fixed with respect to the catheter or capable of motion relative to the catheter. In other embodiments, the therapeutic agent may comprise one or more of an epithelium-destroying agent, a cytotoxic agent, a vector comprising a gene, a cytolytic virus, or a cytokine or hematopoietic growth factor.

In a further embodiment, a kit for destroying mammary gland ductal epithelium is provided that comprises: a duct accessing device comprising: a pair of electrodes, at least one of which is configured to establish electrical communication with the nipple surface; a catheter sized to access the mammary gland; and an agent capable of introduction through the catheter and effective to destroy at least a portion of the ductal epithelium. The electrodes may include a test electrode that is placed at putative sites of mammary gland ducts, and a reference electrode that is placed elsewhere on the body, such as at the base of the breast. The electrodes may function to detect a characteristic electrical value when the test electrode is placed at the site of a mammary gland duct. The characteristic electrical value may be one of a capacitance value, and impedance value, a reactance value, or any other value known in the art. In embodiments, the catheter may be sized so as to be accommodated within a marker tube, which itself is sized to access the duct. In other embodiments, the therapeutic agent may comprise one or more of an epithelium-destroying agent, a cytotoxic agent, a vector comprising a gene, a cytolytic virus, or a cytokine or hematopoietic growth factor. In further embodiments, the kit may also comprise a guidewire, which may be fixed with respect to the catheter or capable of motion relative to the catheter.

Other kit embodiments contemplate the use of a marker dye, and provide a kit for treating or preventing breast cancer, comprising: a nontoxic marker dye or ink that indicates the location of ductal orifices when injected into ducts of the breast; a catheter operable to access the indicated ductal orifices; and a therapeutic agent capable of introduction through the catheter and effective to treat or prevent the cancer. The dye or ink may be toluidine blue, methylene blue, indocyanine green, or any other dye known to be useful for the purpose temporarily or permanently marking the body. In further embodiments, the catheter may be sized so as to be accommodated within a marker tube, which itself is sized to access the duct. In other embodiments, the therapeutic agent may comprise one or more of an epithelium-destroying agent, a cytotoxic agent, a vector comprising a gene, a cytolytic virus, or a cytokine or hematopoietic growth factor. In further embodiments, the kit may also comprise a guidewire, which may be fixed with respect to the catheter or capable of motion relative to the catheter.

Embodiments of the methods include a method of treating or preventing breast cancer, comprising: providing a catheter having an atraumatic tip and a lumen; locating multiple ductal orifices on a nipple using at least one of a duct locating instrument and a compound that visually enhances the location of the ducts; cannulating at least a first ductal orifice by inserting the atraumatic tip into the first orifice; and delivering an agent through the lumen and into the first orifice. The duct locating instrument may comprise one or more of the atraumatic tip, a processing unit, a conductor, and an indicator for indicating when the instrument is located at a duct. In other embodiments, the duct locating instrument may comprise one or more electrodes. The compound may comprise a dye such as toluidine blue, methylene blue, or indocyanine green, or it may comprise another material, compound, or group suitable as an imaging agent. In an embodiment, the compound may be delivered by infusion or injection into the ducts. In other embodiments, the atraumatic tip may comprise a solder ball or a coil at the distal end of the catheter. In further embodiments, the catheter may be sized so as to be accommodated within a marker tube, which itself is sized to access the duct, and after insertion of the atraumatic tip into the ductal orifice, the marker tube may be advanced over the catheter and into the ductal orifice. Following delivery of the agent, the atraumatic tip may be removed from the ductal orifice, leaving the marker tube in place within the orifice. The agent should be one which functions to destroy at least some of the ductal epithelial cells, and in particular embodiments may comprise a cytotoxic agent, a vector comprising a gene, a cytolytic virus, a cytokine or hematopoietic growth factor, or any other agent known in the art to be used for this purpose.

In further embodiments, the method includes cannulating a second ductal orifice using the same catheter, and delivering an agent into the second orifice. In another embodiment, multiple catheters are used to cannulate the multiple ductal orifices, followed by the delivery of the agent to the duct. In other embodiments the multiple catheters are used to cannulate all of the required ducts for the sake of convenience or to avoid the need of using markers. In other words, the methods contemplated include both methods in which a single catheter is used to access multiple orifices for agent delivery, and methods in which a new catheter is used for each ductal orifice that is accessed.

In another embodiment, a method is provided for treating or preventing a condition associated with the epithelial cells along the mammary duct, wherein the method comprises the steps of providing an apparatus for locating and cannulating a ductal orifice, the apparatus comprising a catheter having a distal tip and a lumen, the distal tip comprising a duct probe for locating the ductal orifice; and subsequently cannulating the ductal orifice by inserting the distal tip into the orifice; and administering locally to the ductal epithelium a therapeutic and/or prophylactic agent. In other embodiments, the duct probe comprises at least one electrode. Further embodiments of the method include both methods in which a single catheter is used to locate and cannulate multiple orifices, and methods in which a new catheter is used for each ductal orifice that is accessed, as described above. Further embodiments include repeating the therapeutic and/or prophylactic procedure at selected time intervals, e.g., a single treatment regimen could include a single treatment or treatments spaced days, weeks or months apart and this regimen could be done once or repeated once a month, once every 3 months, once every 6 months, once a year and/or once every 2-10 years, depending among other indices on the presence of marker antigens, genes, clinical state of the patient and the risk/benefit analysis with respect to development of breast cancer.

In another embodiment, a method is provided for destroying ductal epithelium of a mammary gland, comprising: passing an electrical current or potential through the mammary gland and adjacent tissue; identifying multiple ductal orifices of the mammary gland based on measured values of the electrical current or potential; introducing a catheter having a distal tip and a lumen into at least a first ductal orifice; and administering, via the catheter, an agent which destroys epithelial cells. In further embodiments, the step of passing an electrical current or potential through the mammary gland and adjacent tissue is conducted by applying one or more electrodes to the surface of the nipple. In a further embodiment, an electrode is applied elsewhere on the body. In further embodiments, the agent functions to destroy at least a portion of the ductal epithelium, and in particular embodiments may comprise a vector comprising a gene, a cytolytic virus, a cytokine or hematopoietic growth factor, or any other agent known in the art to be used for this purpose. Further embodiments of the method include both methods in which a single catheter is used to access multiple orifices, and methods in which a new catheter is used for each ductal orifice that is accessed.

Certain embodiments of methods that involve identifying the individual ductal orifices comprise engaging at least one reference electrode against a body surface of the mammal (on the breast or elsewhere) and at least one test electrode against a test location on a surface of the nipple. An electrical current and/or potential is applied between the reference electrode and the test electrode, and a characteristic electric signal produced in response to the applied electrical current and/or potential is measured. The measured value is compared to a base or reference value in order to determine whether the test location is at or near a ductal orifice. Usually, the base electrical value will be a predetermined value, e.g., an average or typical value expected for nipple surface locations which do not comprise the ductal orifice. Alternatively, the base electrical value can be determined for each individual patient, e.g., as an average value determined over a large number of locations on the individual nipple surface where particular measured values at particular locations which differ from the average value determined for that patient will then be likely locations for ductal orifices. The reference electrode will usually be located on the body of the mammal at a location remote from the nipple surface, for example, at the base of the nipple, at the base of the breast, or elsewhere on an abdominal or chest surface of the mammal. Alternatively, in some instances, it will be possible (although generally not preferred) to measure the electrical characteristics between adjacent, laterally spaced-apart locations on the nipple surface in a bipolar manner. Such an approach will determine the surface electrical characteristics of the nipple. Generally, however, it will be preferred to measure electrical characteristic determined from the nipple surface through the “body” of the nipple to an external surface location remote from the nipple.

The test electrode(s) can be applied to the nipple surface in a variety of configurations. Most simply, the test electrode can comprise a single electrode element which may be contacted sequentially at multiple points on the nipple surface, either manually or using an automated positioning system. Electrical characteristic values can then be collected or displayed in a variety of ways. Electrical impedance or other values can be displayed to the person running the test. The person can then observe the value and note changes in electrical characteristics which denote the likelihood of the presence of a ductal orifice. Such manual or semi-automated approaches will be particularly valuable in conjunction with visual confirmation of the presence of an orifice. For example, detection of a change in the electrical characteristics may alert the person running the test to visually scan that area of the nipple more carefully for the presence of the orifice. Alternatively, the individual may simply collect data which is then transmitted to a data collection and analysis system, typically a digital analyzer such as a personal computer.

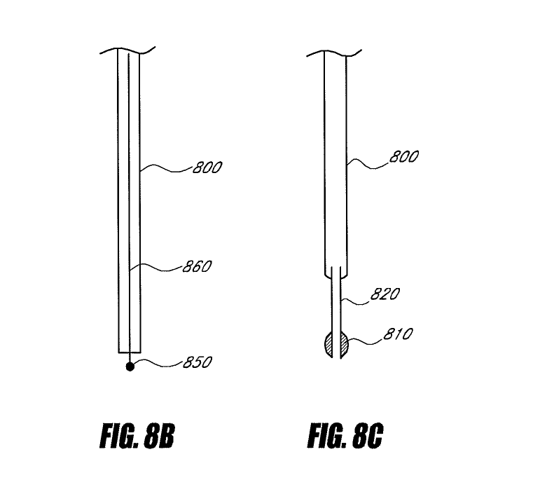

The electrical current and/or potential applied between the reference electrode(s) and test electrode(s) may comprise direct current, alternating current, high frequency alternating current, or any other type of electrical signal which can be applied to the patient in a safe and generally painless manner and produce a responsive signal that can be detected and which will vary depending on the proximity of a ductal orifice. In one embodiment, the electrical signal is a relatively low frequency signal, e.g., having a frequency in the range from 1 kHz to 10 kHz. The responsive signal measured is electrical impedance. In another embodiment to be described in greater detail later in connection with FIGS. 9-17, two or more frequencies are used.

The electrical impedance or other characteristic signal is usually measured using the reference electrode(s) and test electrode(s). For example, in the case of electrical impedance, measurements can be made based on the known voltage and current being applied between the test electrode and reference electrode. Other conventional systems for applying electrical energy and measuring characteristic responses are well-known in the patent and medical literature.

In certain embodiments, the duct probe may comprise means other than electrical for locating (identifying) the ductal orifice. Such alternative means may include for example, optical means, temperature means, mapping means, expressed fluid/dye means, etc. Optical means may include miniaturized endoscope type devices that employ an optical fiber imaging system like the optical imaging guidewire of Mihalcik et al. US2004/0034311A1, or a magnifying device for surveying the nipple topography and localizing a ductal orifice of Hung et al., U.S. Pat. No. 6,328,709; the disclosures of which are incorporated herein in their entirety by reference.

Also disclosed are kit embodiments including at least one reference electrode and one test electrode capable of being engaged against the nipple and other body surfaces as described above. Usually, the test kits will further comprise instructions for use setting forth any of the methods described above. Additionally, the kits may include elements for accessing a ductal orifice after it has been identified, elements for marking a ductal orifice after it has been identified, conductive gels for use in combination with electrode arrays, and the like. The kits may further comprise packaging for holding the kit components, usually in a sterile manner, instructions for use may be printed on separate package inserts, and/or be printed in whole or in part on the packaging itself. The kits may also comprise a therapeutic or prophylactic agent for administration to the ductal epithelium, such as those described below.

Breast augmentation or reconstruction of the breast requiring an implantable medical prosthesis has become fairly common practice in the art of plastic and reconstructive surgery. Typical permanent prostheses, which are often selected for these procedures, include round silicone shells, or envelopes, pre-filled with silicone gel or filled at the time of surgery with normal saline solution.

Unfortunately, there still exist many problems with breast augmentation surgery. Besides the general undesirability of an invasive surgical procedure, current implants can rupture, deflate or leak. Silicone-filled implants may release this foreign substance into the body. Saline-filled implants are considered preferable in that breakage will release only a sterile saline solution into the body. There is concern, however, that the saline-filled implants could support the growth of fungus and certain bacteria. Rupture or leakage would then release these potentially harmful organisms into the patient's body.

Besides implant failure and release of potentially toxic fillers, surgeons often observe undesirable alterations in the patient's breast shape during post-operative follow-up, including e.g., signs of skin and/or soft tissue deformation, commonly known to those skilled in the art as prosthesis wrinkling, knuckling or scalloping. These adverse effects usually occur at the upper or lower pole of the prostheses, along the perimeter of the prosthesis shell or at the base, i.e. the inferior portion closest to the infra-mammary fold, and become more evident when the recipient changes her anatomical position. Moreover, with the patient in an upright position, these unstable prostheses have been known to collapse or fold in the upper pole and knuckle in the lower pole, further increasing risk of deformed breast shape. Medical prostheses have been proposed in an attempt to eliminate these clinical problems, but adverse alterations in breast shape continue to exist.

Accordingly, there remains a substantial unmet need in the area of breast aesthetics for procedures that provide less invasive breast augmentation without serious health risks and adverse alterations in breast shape.

The following preferred embodiments and examples are offered by way of illustration and not by way of limitation.

The method is practiced to identify and access a milk duct orifice or orifices on a mammalian nipple surface. The mammal can be any mammal, e.g., a mammal having breasts with nipples, thus including e.g., humans, primates, pigs, dogs, cows, horses, rabbits, rats, cats and mice. Generally, the purpose of locating the ductal orifice is so that the duct can be accessed through the orifice, e.g., for delivering an agent to the duct and/or retrieving cellular and other materials from the duct. The agent can be, for example, saline for washing the duct and retrieving cells from the duct. In a further embodiment, the agent is a therapeutic or prophylactic agent.

The method comprises new uses for electrical technology for the purpose of locating and accessing ductal orifices. An electrical circuit is created using a reference electrode and a test electrode and an apparatus capable of generating an electrical current and/or potential between the two electrodes, or between more than one electrode and a reference electrode. The reference electrode is placed on the body someplace other than the location where the test electrode is placed. The test electrode is generally placed on the nipple surface in order to attempt to identify the location of a breast duct orifice. A characteristic electrical signal is generated upon placement of the test electrode on the nipple surface. The characteristic electrical signal can be e.g., values of impedance, resistance, capacitance, inductance or reactance, or any electrical signal capable of conveying information to differentiate a location of the test electrode at a ductal orifice or not at a ductal orifice on a nipple surface.

The characteristic electrical signal can be compared to a reference value in order to identify the likelihood that the test location is a ductal orifice or not. The reference value can be predetermined, and thus the reference value can be e.g., the results of a cumulative survey of values of the particular electrical signal for ductal orifices or non-ductal orifices in order to help determine when a test electrode is most likely at a ductal orifice. Thus, e.g., a reference value might indicate a value below which a test electrode is most likely at a ductal orifice and above which a test electrode is most likely not at a ductal orifice. A reference electrode is located at a different location on the body than the test electrode. For example, the reference electrode can be on the body of the mammal being studied at a location including e.g., the base of the breast being studied, the base of the nipple being studied, the surface of the nipple being studied (albeit at a different location on the nipple surface than the test electrode), the areola of the nipple being studied, or the trunk of the mammal. The reference electrode can be affixed to its location on the mammalian body in any way that is safe to the patient and provides continuous contact with the skin surface, e.g., an adhesive pad comprising the electrode that contacts the skin surface. The reference electrode can have a lead connected to it that provides connection to the machine source that both generates a potential and measures the characteristic electrical signal. The reference (or return or emitting) electrode can be positioned as described in U.S. Pat. No. 5,143,079, WO 96/12439, or U.S. Pat. No. 5,810,742 (incorporated herein by reference in their entirety) especially where descriptions are refined for studies of the human breast; which patent disclosures are incorporated herein in their entirety.

A test electrode is placed on the nipple surface. In an embodiment, a single test electrode is used, and in that case the test electrode is placed sequentially at different locations on the nipple surface and a value of the characteristic electrical signal is taken at each different location relative to a reference value, which can be a predetermined value, and using also a reference electrode placed at a reference point on the body of the mammal. The test electrode can be set up to “scan” the surface of the nipple and thus to contact in fast succession multiple locations on the nipple surface. The scanning process provides multiple electrical signals so that the test electrode can test a large portion of the nipple surface rapidly. An identification signal such as a visual or auditory signal can be established when the test electrode has identified a location on the nipple surface likely to be a ductal orifice.

Alternatively, the test electrode can comprise multiple electrodes placed at the surface of the nipple. In the case of multiple test electrodes, a potential is applied between the reference and test electrodes, where each test electrode participates in a circuit with the reference electrode in succession.

In a simple format, one embodiment of the invention contemplates a method of identifying and accessing a ductal orifice by applying an electrical potential and/or current to the mammalian breast using a reference electrode placed at a reference point on the body of the mammal, and then applying a test electrode to a first and then a second and/or subsequent locations on the nipple surface and measuring a first and then a second value of characteristic electrical signal. If the test electrode is a single electrode then the test electrode is placed at the first location and then physically picked-up and moved to a second location on the nipple surface for the second location. If there are multiple test electrodes sitting on the nipple surface, a switcher can switch the potential and/or current successively from a first electrode to a second electrode thus generating a first and second value of a characteristic electrical signal from a first and second location on the nipple surface. Ultimately, a comparison of values between the first and second locations can be generated such that a ductal orifice is identified upon detection of a differential value of characteristic electrical signal. Alternatively, and preferably, any given electrical signal at a given test location is compared to a predetermined reference value for making an absolute determination of likelihood that the electrode has identified a ductal orifice.

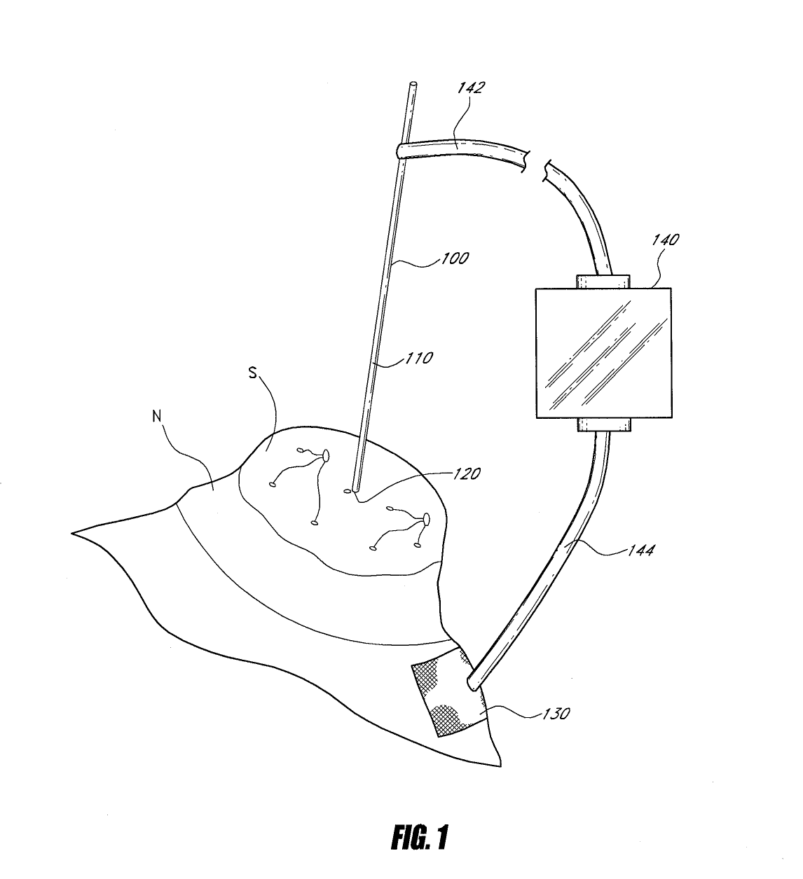

FIG. 1 illustrates a first embodiment of an apparatus of the invention. An electrode-containing catheter structure 100 is adapted for manual manipulation, e.g., it may be in the form of simple “pencil” structure having a shaft that can be manually grasped. Although a catheter is employed in the present embodiment, other accessing elements are known that are capable of accessing the ductal orifices of mammary glands. Examples thereof include, for example, appropriately sized cannulas. The catheter structure 100 is sized to permit it to be advanced within a ductal orifice. The outer diameter of the catheter is preferably within a range of 0.008-0.040 inches. More preferably, the outer diameter is within a range of 0.010-0.032 inches. Most preferably, the outer diameter is within a range of 0.012-0.030 inches. Furthermore, the catheter preferably has a length within a range of 0.25-20 inches. More preferably, the length is within a range of 0.75-18 inches. Furthermore, the catheter structure 100 is provided with a lumen 110 that allows a flow of an agent, such as a therapeutic or prophylactic agent, through the lumen 110 to a distal end of the catheter structure 100. The distal end of the catheter structure 100 forms a test electrode probe 120 that can be engaged directly against the surface S of the nipple N. The test electrode probe 120 may comprise a coil, a ball or other known type of electrode and has an atraumatic distal end that comes into contact with surface S. A separate surface or reference electrode 130 is provided and adapted for attachment to a body surface remote from the nipple. As shown in FIG. 1, the reference electrode 130 is mounted on the breast just below the nipple N. Reference electrode 130 can, of course, be mounted at any of the other body locations described elsewhere herein. The test electrode probe 120 is connected to an analyzer 140 via cable 142. Similarly, the reference electrode 130 is connected to the analyzer 140 via a cable 144. The analyzer 140 may have a wide variety of configurations as described elsewhere herein. Typically, the analyzer 140 will include at least a source of electrical current and circuitry for analyzing that current. The analysis circuitry may be included wholly within a single analyzer 140, or the analyzer 140 may be adapted to interface with other analyzer circuitry, such as in the form of a personal computer or other digital, programmable analyzer. Usually, at least the test electrode probe 120 and reference electrode 130 will be disposable and thus detachable from their respective cables 142 and 144. Alternatively, the entire cable structures may also be disposable and thus detachable from the analyzer 140 or the entire cable structures and analyzer will be disposable.

The test electrode probe 120 of FIG. 1 will usually be used manually, where an operator successively engages the test electrode probe 120 against different test locations on the surface S of the nipple N. At each location, an impedance or other electrical characteristic value, such as capacitance, is measured. The measured value may be displayed, recorded or both, and the acquired data is eventually relied on to determine those locations on the nipple surface which are most likely to comprise an individual ductal orifice O.

When the characteristic electrical value indicates that the test electrode probe 120 is located at a ductal orifice, the operator may then advance the catheter structure 100 through the ductal orifice and into the duct, thus accessing the duct. Liquid or gaseous agents may then be supplied from sources not depicted in FIG. 1 through the catheter into the interior of the duct for treatment or prophylaxis of diseases arising in the ductal epithelium.

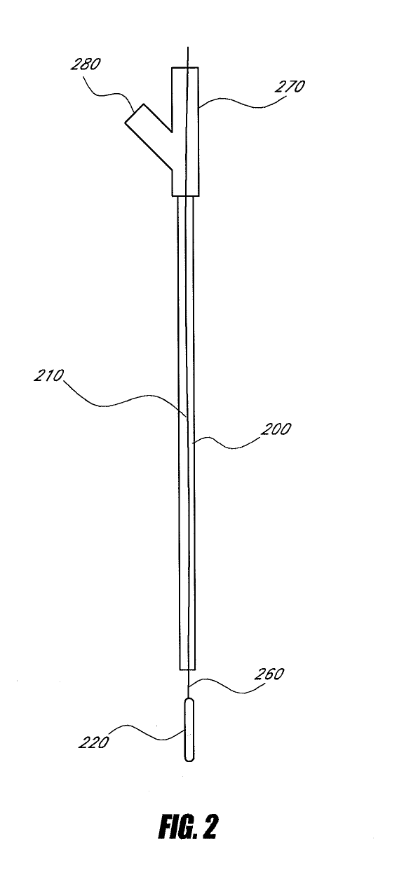

FIGS. 2-7 show alternative embodiments of the catheter and electrode structure. Although these embodiments focus on catheters, other known accessing elements, such as cannulas, may also be employed, as described above.

FIG. 2 shows a schematic cutaway view of an embodiment of the catheter structure 100 of FIG. 1. In this embodiment, a probe electrode 220 is mounted on a guidewire 260 that is accommodated within the lumen 210 of the catheter structure 200. The distal end of the electrode 220 forms an atraumatic end that comes into contact with the surface of the nipple. The lumen 210 is sized to admit guidewire 260 therein while simultaneously permitting the passage of therapeutic or prophylactic agents therethrough to the distal end of the catheter structure 200. The catheter structure 200 is also is provided with a hub 270 that allows communication via a syringe port 280 with a syringe or other device for supplying a therapeutic or prophylactic agent. The guidewire 260 may be affixed to the hub 270 via a glue joint or the like so that the guidewire does not move relative to the catheter structure 200. In this case, the guidewire advances into the interior of a duct after the ductal orifice thereof is located, and the prophylactic or therapeutic agent is administered to the interior of the duct via the catheter structure 200 while the guidewire 260 is in place. Alternatively, a fitting may be employed in hub 270 that permits relative motion of the guidewire 260 and the catheter structure 200. This may be accomplished by employing a Touhy Borst adapter or the like in hub 270. When such a structure is employed, the guidewire may be partially or completely removed from the flow path of the agent through the lumen 210 of the catheter structure 200 before the administration of an agent via the syringe port 280.

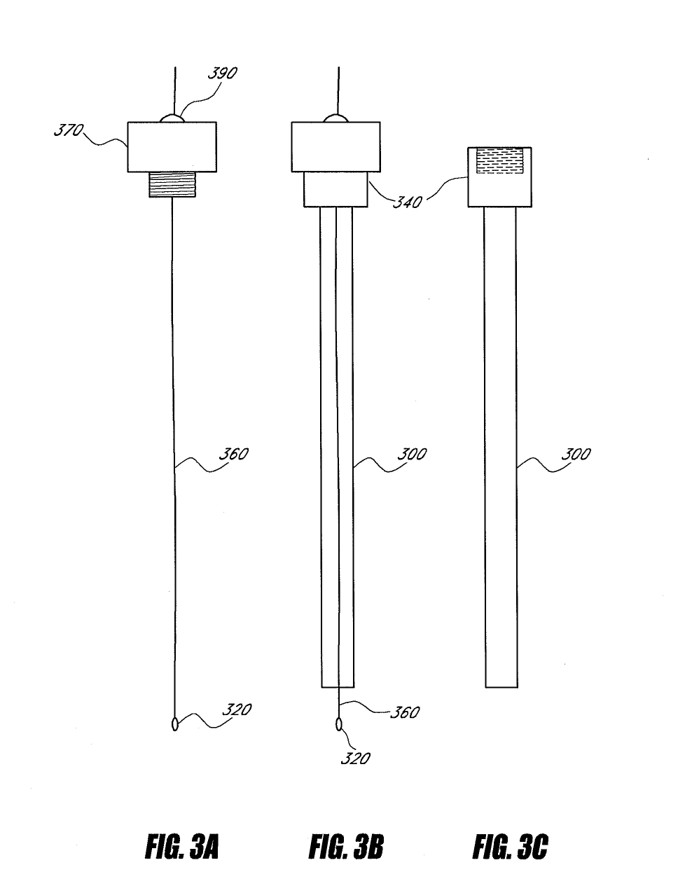

A third alternative embodiment of the catheter and electrode is illustrated in FIG. 3. As shown in FIG. 3A, a guidewire 360 is attached via a glue joint 390 to a threaded hub 370. The distal end of the guidewire 360 forms an atraumatic tip to which an electrode 320 is mounted so as to achieve electrical contact with the surface of the nipple when the distal end of the guidewire is brought into contact therewith. The electrode 320 may comprise a coil or any other electrode configuration known to those of skill in the art.

In use, the assembly containing the guidewire 360 and the threaded hub 370 is inserted into a catheter 300 having a thread 340 on its proximal end that is formed to screwably engage with the thread on the threaded hub 370. Before use, the catheter 300 is screwed into the threaded hub 370 so as to achieve a stable connection. This is shown in FIG. 3B. The length of the guidewire 360 extending distally of the threaded hub 370 and the length of the catheter 300 are preferably selected so that at least the entire length of electrode 320 protrudes beyond the distal end of the catheter 300 when the catheter 300 is engaged with the threaded hub 370. Once the threaded hub 370 and the catheter 300 are engaged, the electrode 320 is brought into contact with the surface of the nipple. When the characteristic electrical value measured indicates that the electrode is located at a ductal orifice, the combination of the catheter 300 and guidewire 360 is advanced into the duct. Thereafter, the threaded hub 370 to which the guidewire 360 is affixed is unscrewed from the catheter 300 and the guidewire 360 is removed, leaving the catheter 300 available as a conduit for the introduction of a prophylactic or therapeutic agent into the interior of the duct, as shown in FIG. 3C.

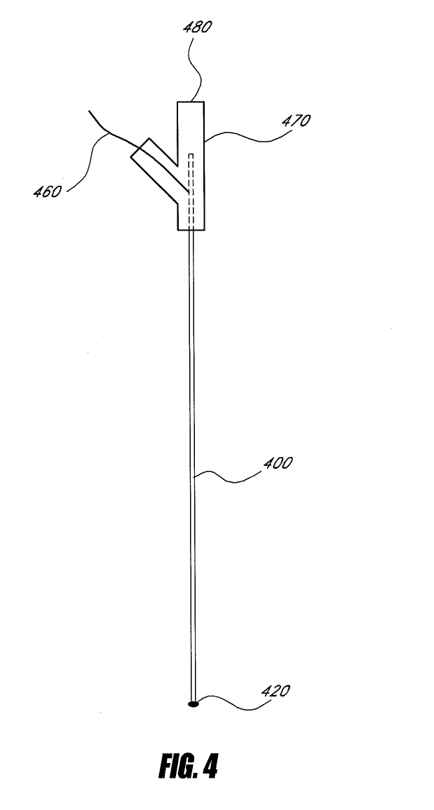

A fourth alternative embodiment is illustrated in FIG. 4. In this embodiment, the functions of the catheter and electrode are combined in a flexible metal hypotube 400. The tube 400 has very thin walls and comprises, for example, stainless steel or Nitinol. The distal end of the tube 400 is affixed to a solder ball 420 which renders the distal tip relatively atraumatic. The tube 400 is coated with an electrically insulating coating along its entire length with the exception of the solder ball 420 at the distal end. As shown in FIG. 4, the tube 400 is housed within a hub 470 having a syringe port 480 sized to fit a syringe (not depicted) for the introduction of an agent into the interior of the tube 400. The hub 470 also accommodates a wire 460 which allows electrical contact between the tube 400 and the remainder of the duct detection apparatus.

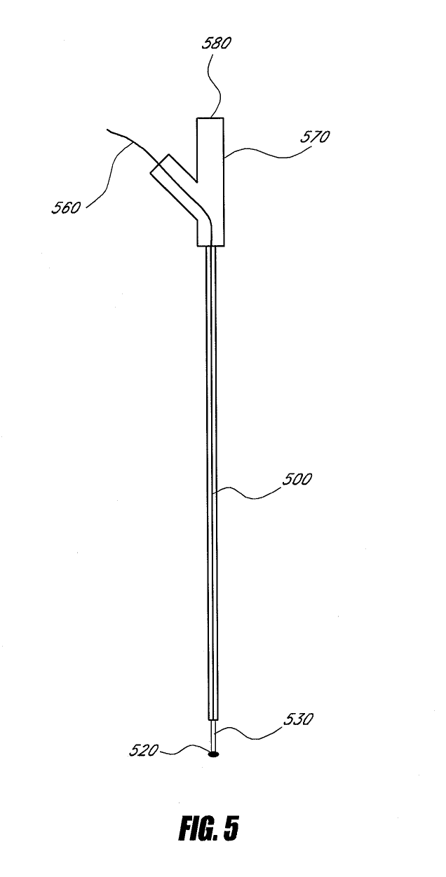

A fifth embodiment is illustrated in FIG. 5. In this embodiment, a metal hypotube 530 is provided at the distal end of an ultraflexible catheter 500. The catheter 500 may comprise a polymer or other material that is biocompatible and provides a high degree of flexibility. The distal end of the tube 530 terminates in a solder ball 520 that functions as an atraumatic electrode to make contact with the surface of the nipple. The tube 530 is in electrical contact with a wire 560 that provides contact with the remainder of the duct detecting apparatus. The catheter 500 is connected to a hub 570 that is provided with a syringe port 580 sized to fit a syringe (not depicted) for the introduction of an agent into the interior of the catheter 500.

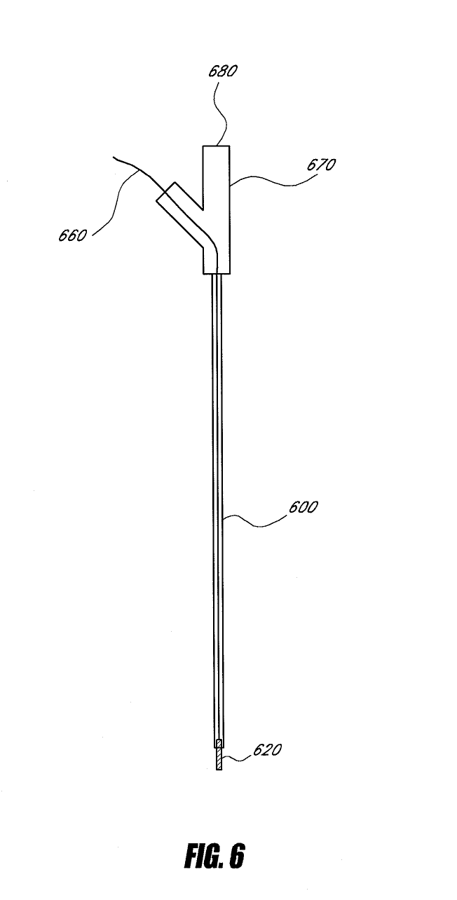

A sixth embodiment is illustrated in FIG. 6. In this embodiment, a catheter 600 comprising a polymer is provided. A coil 620 is provided within the distal end of the catheter 600 in such a manner that a portion of the coil 620 protrudes beyond the distal end of the catheter 600. The distal end of the coil 620 is formed with a blunt atraumatic tip to contact the surface of the nipple without causing damage thereto. The pitch of the coil 620 is set so that an agent provided through the lumen of the catheter 600 can exit through gaps in the coil 620. The coil 620 is in electrical contact with a wire 660 that provides contact with the remainder of the duct detecting apparatus. The catheter 600 is connected to a hub 670 that is provided with a syringe port 680 sized to fit a syringe (not depicted) for the introduction of an agent into the interior of the catheter 600.

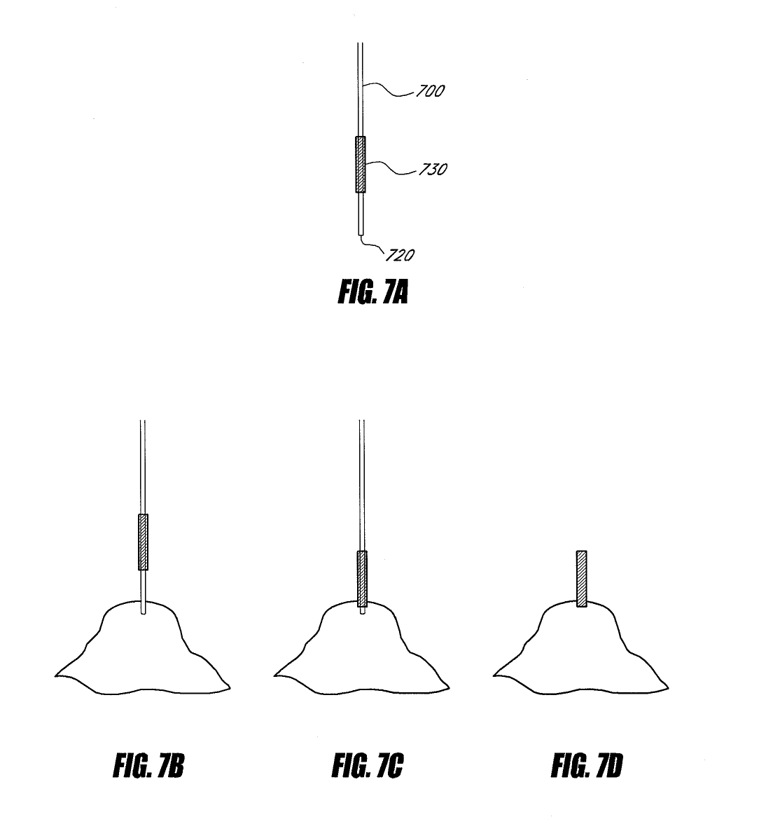

A seventh embodiment is illustrated in FIG. 7. In this embodiment, a marker tube 730 is advanced over a standard catheter body 700 having an electrode 720 formed at the distal end thereof. As shown in FIG. 7A, the marker tube 730 is sized so as to fit around the catheter body 700. When the ductal opening is detected using electrode 720, the catheter is inserted into the duct, as shown in FIG. 7B. As the catheter body is further inserted into the duct, the marker tube 730 enters the ductal opening, as shown in FIG. 7B. The outer diameter of the marker tube 730 is set so that it will become lodged in the ductal opening and remain there when the catheter is removed, as shown in FIG. 7C. The marker tube facilitates repeated access to the duct, as when different tools are to be used in treating the duct or when the duct is to be repeatedly treated. The marker tube 730 may be made rigid, so as to maintain its shape and a defined lumen when the catheter body 700 is removed. Alternatively, a thin-walled marker tube 730 may be employed that will collapse when the catheter body is removed.

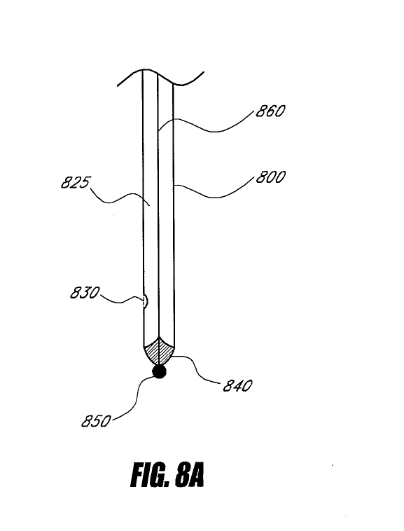

An eighth embodiment is shown in FIG. 8A. In this embodiment, the catheter 800 comprises a polymeric material. In a preferred embodiment, the polymer material is a block copolymer. In a particularly preferred embodiment, the block copolymer material is PEBAX® (polyether block amide). As shown in FIG. 8A, a solid or hollow conducting element 860 is accommodated within the catheter 800. The outside diameter of the catheter may be constant along the distal region or it may vary, e.g., tapered, such that it preferably exhibits flexibility and stiffness consistent with a cannulating catheter. The conducting element 860 may comprise any metal or conducting material, e.g., those commonly employed in guidewire applications. In a preferred embodiment, the conducting element 860 comprises a nickel-chromium-molybdenum steel that is cold worked and has a high tensile strength. In a particularly preferred embodiment, the conducting element comprises Hyten M steel. In a preferred embodiment, the conducting element is covered with a polymeric film. In a preferred embodiment, the film comprises PET (polyethylene terephthalate). In a particularly preferred embodiment, the PET film is shrinkwrapped onto the conducting element 860 before the conducting element 860 is placed, e.g., coaxially, into the catheter 800. The presence of this film substantially prevents contact between the conducting element 860 and any fluid, such as a therapeutic and/or prophylactic substance, within the infusion space 825 of the catheter 800.

As shown in FIG. 8A, the distal end of the conducting element 860 comprises an atraumatic member, e.g., a metal ball 850, which may be integral with or otherwise attached to the conducting element, e.g., by solder or welding. In a preferred embodiment, the ball comprises laser-treated metal. The ball preferably has a diameter within a range of approximately 0.01-0.0001 inches. More preferably, the ball has a diameter within a range of approximately 0.005-0.0005 inches.

Furthermore, the atraumatic member 850 is connected to catheter 800 via a polymeric bond 840. In a preferred embodiment, the bond is formed by heating the distal end of the catheter to form a bond between the polymeric material of the catheter 800, which comprises PEBAX in a preferred embodiment, and the polymeric film covering conducting element 860, which film comprises PET in one preferred embodiment. The bond between the polymeric materials seals the distal end of catheter 800. To permit exit of fluid, such as a therapeutic solution, introduced at the proximal end of catheter 800, an infusion port 830 is provided along the distal region of the catheter 900, preferably as illustrated in FIG. 8A, in the vicinity of the distal end of the catheter 800. The infusion port 830 is particularly advantageous where, as in this embodiment, the catheter is sealed at the distal end; however, one or more such ports may be advantageously provided along the distal region of the catheter in any of the embodiments of the present invention, and such modification of the disclosed embodiments is specifically contemplated.

A further embodiment is shown in FIG. 8B. In the embodiment shown in FIG. 8B, the catheter 800 is formed so that the physical properties thereof change over the length of the catheter. In one embodiment, for example, the thickness of the walls of the catheter 800 vary from the proximal end to the distal end thereof. In a preferred embodiment, the walls are thickest in the vicinity of the proximal end of the catheter, and become progressively narrower toward the distal end thereof, where the walls are at their thinnest. In a further embodiment, the outer diameter of the catheter is greatest at the proximal end thereof, and progressively decreases in the direction of the distal end. In a particularly preferred embodiment, the thickness of the catheter walls also decreases with the decrease in the outer diameter of the catheter. These characteristics are suggested in FIG. 8B by an exaggerated tapering of the catheter 800. In such an embodiment, the end of the catheter may be sealed and one or more lateral infusion ports provided, as shown in FIG. 8A, or alternatively, the distal end of the catheter may be left open as illustrated in FIG. 8B, wherein the conducting element 860 comprises a conventional guidewire modified to comprise an atraumatic member 850. In one embodiment, the wire may be tapered.

In a further embodiment, as shown in FIG. 8C, the catheter 800 terminates in a metal hypotube 820. In a preferred embodiment, the hypotube comprises a nickel-titanium (NiTi) alloy. In a particularly preferred embodiment, a NiTi ball 810 is formed on the distal end of the hypotube 820 to achieve an atraumatic tip. The hypotube may be connected to the proximal end via a conducting wire or the hypotube may extend through the length of the catheter.

A side view of an embodiment of a structure that may be employed at the proximal end of the catheter 800 is shown in FIG. 8D. As shown in FIG. 8D, the proximal end 870 of catheter 800 comprises two tubes 875 and 880 that project from the catheter 800. The tubes 875 and 880, as well as an anchoring base plate 890 are preferably part of a single integrally molded structure. In other embodiments, the tubes 875 and 880 may be attached to a common anchoring plate 890. In one embodiment, the tube 875 is connected to the catheter 800 at a point that is more distal than the point at which tube 880 is attached to the catheter, although the relative orientation of the tubes 875 and 880 may be reversed. Tube 875 terminates (as shown) or is otherwise operably coupled with an infusion port 877 at the proximal end thereof, and may be used for the introduction of fluids, such as therapeutic and/or prophylactic solutions, into the infusion space 825 of the catheter 800. The tube 875 is fluidly coupled with the infusion space 825. The infusion port 877 is preferably configured to accept conventional infusion devices, e.g., a conventional female luer lock coupling which is configured to fluidly couple to the tapered male luer lock coupling on a typical syringe. Tube 880 accommodates the conducting element (e.g., 860 from FIG. 8A) or a metal tube 885, such as a hypotube, that is connected to the mechanism used to probe the surface of the breast tissue for electrical signals characteristic of a ductal opening, as described elsewhere (such as the conducting element 860 (FIG. 8A), the guidewire embodiment of the conducting element 860 (FIG. 8B) or the hypotube 820 or wire extending from the hypotube 820 (FIG. 8C). In some embodiments, a substance such as a therapeutic solution may also be accommodated within this metal tube, e.g., where the hypotube is both the conducting element and the infusion space. To facilitate manipulation of the metal tube 885 and catheter 800, the metal tube 885 is accommodated within an insulating sleeve 895. This insulating sleeve 895 surrounds the metal tube 885 and protects the user against effects of temperature or electric currents that may pass through the metal tube 885.

In one preferred embodiment, the anchoring base plate 890 has a means disposed on one surface of the plate for anchoring the plate to the patient, e.g., an adhesive pad, such as those typically used for affixing ECG electrodes to a patient's skin. Any adhesive structure known in the art to be suitable for removable affixing to a patient's skin may be used, including e.g., tape. Since a nipple has a plurality of milk ducts, it may be advantageous to undertake a step-wise procedure, wherein the physician or technician: (1) cannulates one duct with a first catheter, while the conducting element 860 of the first catheter is operably coupled to an analyzer for detecting the characteristic signal and providing the user with a audible and/or visual signal indicating that the distal tip of the conducting element is located on a milk duct orifice; and then (2) affixes the proximal end of the first catheter (e.g., the anchoring base plate 890) to the patient. This procedure is then repeated using consecutive catheters until all of the milk ducts in the nipple have been cannulated and all of the proximal ends of the consecutive catheters are available at relatively fixed and identifiable locations on the patient's body for sequential infusion of therapeutic and/or prophylactic agents via the respective infusion ports 877.

The reference electrode may be on the nipple surface also, or may also be at the base of the breast or at the base of the nipple or at any other suitable reference point on the mammal being studied.

The reference and test electrodes can be made of e.g., silver, silver/silver chloride, platinum, titanium, stainless steel or other conductive material suitable for use as an electrode contacting a skin surface. Electrodes can be made as described in U.S. Pat. No. 5,217,014, U.S. Pat. No. 5,660,177, and WO 96/12439 and U.S. Pat. No. 5,810,742 (incorporated by reference herein in their entirety) with further modifications as described herein, including modifications to suit the particular purpose of an embodiment of the invention, e.g., to identify ductal orifices locations on a nipple surface; there references are incorporated herein in their entirety by reference. The test electrodes and the reference electrode can have lead (insulated conductive wire) to a machine capable of generating an electrical potential or current in an electrical circuit for generating the potential and measuring the characteristic electrical signal values. The machine capable of generating an electrical potential or current in an electrical circuit has the ability to generate potentials at various frequencies for making these measurements. One type of machine for generating the potential and measuring the characteristic electrical signal values can be an LCR machine (LCR stands for admittance, capacitance and resistance). The electrode wire size can be e.g., in the range of 1 μm to 1 mm diameter, e.g., a range of about 50 μm to 500 μm in diameter.

Measuring the characteristic electrical signal can comprise making measurements at a specific frequency or at a range of frequencies, using e.g., a machine capable of generating an electrical potential or current in an electrical circuit. The measurements can be made at a range of frequencies and the range can be predetermined. For example, a frequency can be in the range of 100 Hz to 1000 kHz. For a predetermined range, several frequencies can be selected, e.g., 1000 Hz, 10 kHz, 100 kHz, and 1000 kHz and tested in different locations on the surface of the nipple.

Thus, e.g., location A is probed at 2 to 10 frequencies, and location B is probed at the same 2 to 10 frequencies, and the characteristic electrical signal values at each location for each frequency can be compared. The range of frequencies used can be a random range or a predetermined range of frequencies. With regard to an embodiment having multiple test electrodes, the machine capable of generating an electrical potential or current in an electrical circuit can be connected to a switcher for relaying the potential and taking measurements at each electrode. The switcher can be connected to a machine for analyzing the electrical signals, e.g., a central processing unit (CPU) for analyzing (and capturing and storing) the data retrieved from the characteristic electrical signal measurements.

After placement of the reference electrode and the test electrode on their respective parts of the mammal to be analyzed, (e.g., the reference electrode is placed at the base of the breast, and the test electrode is placed at the nipple surface) a reference potential can be established between the two electrodes. Although an embodiment of the invention is not limited to theories of how the system works, it is proposed that a potential between a reference electrode and a test electrode will preferentially travel through the breast ducts with a lower characteristic electrical signal value. The electrodes can be connected to leads that connect to a machine (or analyzer), for example a machine capable of generating an electrical potential or current in an electrical circuit, capable of establishing a potential between the electrodes and of measuring characteristic electrical signal within the circuit.

The machine can be one essentially as described in U.S. Pat. No. 5,143,079, WO 96/12439, U.S. Pat. No. 5,810,742, U.S. Pat. No. 5,678,547, U.S. Pat. No. 4,458,694 or U.S. Pat. No. 4,291,708 (incorporated by reference herein in their entirety); which are incorporated herein in their entirety by reference. The machine can be capable of generating an electrical potential, and can also include a machine for analyzing the electrical signal, (e.g., a central processing unit (CPU)) for making analysis of the measurements that are taken from the potential generated between two electrodes. Characteristic electrical signal is a value that can be measured by virtue of the potential difference between the two electrodes. Other parameters that can be derived from a characteristic electrical signal measurement including resistance or capacitance can also be measured and used to compare the probe locations. Thus, for example, point A on the nipple surface may have a lower characteristic electrical signal measurement than point B on the nipple surface, indicating the likelihood that point A is a ductal orifice. It is also important to note that a ductal orifice may be indicated by higher characteristic electrical signal where the ductal orifice has a keratin plug, as keratin plugs will generally provide for higher characteristic electrical signal of the potential. One embodiment of the invention accordingly also provides that the nipple can be dekeratinized with an appropriately effective dekeratinizing agent before the electrical measurements are taken. Dekeratinizing agents include those known in the art, e.g., acetic acid at a dekeratinizing strength, empigen, cerumenex, and preparative agents containing alcohol.

To identify a ductal orifice, a test electrode may be moved across the nipple surface, testing for areas of characteristic electrical signal variation, including areas of low characteristic electrical signal and high characteristic electrical signal. At a particular area of low characteristic electrical signal, for example, (if low signal indicates a ductal orifice) the catheter can be inserted within a ductal orifice, and the catheter may be used to further evaluate the duct to determine if it is in need of treatment. As described above, in one preferred embodiment, once an ductal orifice has been cannulated by a catheter, the proximal end of that catheter may be removably affixed to the patient, and another catheter is then used to probe for and cannulate another ductal orifice, until all of the ductal orifices have been cannulated and all of the separate catheters are affixed in an orderly fashion on the patient for subsequent lavage or infusion of active agents. Alternatively, a marker tube may be inserted into the duct, as described above and shown in FIG. 7, and the procedure may be continued until all ducts have been located. The placement of the marker tube provides the practitioner with the opportunity to access the duct later, after the scanning of the nipple surface is complete and all the ducts have been identified. The marker tubes placed in the ducts also provide an opportunity to take a photograph of the nipple with marker tubes extending from it for recording the locations of the ductal orifices.

The ductal orifices can be imaged as described by a photograph of a visual marker such as an inserted marker tube, a tattoo or other mark at the orifice on the surface of the nipple. A current sensitive film (e.g., a liquid crystal film) can be placed on the nipple to show a map of the characteristic electrical signals on the nipple surface indicating areas of likely location of ductal orifices. The electronic values can be printed from the computer. Evidence of any characteristic electrical signals from the nipples surface can be imaged electronically, by photo, and recordation of numerical values corresponding to the signals.

The machine connected to the leads may also contain a machine for analyzing the electrical signal, (e.g., a central processing unit (CPU)) for processing the electrical signals generated by the measured values of characteristic electrical signals received from the test probe at the nipple surface. The machine for analyzing the electrical signal can generate electronic signals that provide an image or map of the nipple surface. Electronic imaging can provide a practitioner with a real time image to use in accessing the ducts. An electronic image can also provide the opportunity for generating a printed image of the nipple surface that indicates the location of the ductal orifices for the patient's records. The machine for analyzing the electrical signal can also capture the characteristic electrical signal values and store them for later access. The practitioner can work in real time from the electronic image, or can use the printed image to provide a map to the ductal orifices.

The ducts can be accessed for any purpose, including providing a medical treatment or procedure comprising therapeutic, prophylactic, diagnostic or prognostic purposes. For example, tissue or fluid can be removed from the duct, or agents can be delivered to a duct or ducts, as described in greater detail below. The test electrode can be operated while access of the ductal orifice is desired. Thus, upon identification of a ductal orifice, access can be made immediately using a catheter or cannula, as described above, or the ductal orifice can be marked with a marker tube. The purposes of access can include, e.g., removing fluid or administering an agent or marking the ducts as with marker tubes, or other analysis or manipulation of the duct. The probe can also be an imaging probe where desired, for example a ductscope or other imaging device capable of penetrating a breast duct once accessed.

Characteristic electrical signal measuring devices can be made essentially as described in WO 96/12439 to TransScan, U.S. Pat. No. 5,810,742 also to TransScan, U.S. Pat. No. 5,143,079 to Yeda Research and Development Co., U.S. Pat. No. 5,678,547 to Biofield Corp., and U.S. Pat. No. 5,660,177 to Biofield Corp; all of which are incorporated herein by reference in their entirety. Modifications can be made to the devices described in these patents and publications and herein, including adaptations made in order to read electrical potentials at the surface of a nipple, particularly those at ductal orifices. Thus, electrodes are placed at some point on the body, e.g., at the base of the breast, and on the nipple surface in order that characteristic electrical signal between these two points is measurable after a potential is applied between the electrodes completing a circuit. The devices include a reference electrode for placement at a point on the body other than the location of the test electrode, a test electrode for placement on the surface of the nipple, leads from each electrode to a machine capable of generating an electrical potential between the two electrodes and of measuring a value of a characteristic electrical signal existing at a given test spot, e.g., a machine capable of generating an electrical potential or current in an electrical circuit.

Such a device used in the method of identifying ductal orifices is used to take multiple comparable measurements of characteristic electrical signal at various points on the nipple surface. The ductal orifices are identified, e.g., by a characteristic electrical signal. Thus where a characteristic electrical signal is detected, a ductal orifice on the nipple surface can be identified. The detection is made based on characteristic electrical signal measurements at that spot as compared preferably to a reference value (e.g., a value established based on a survey of likely values for ductal orifices on nipple surfaces), also with reference to a potential and/or current created in a closed circuit with the reference electrode. Ultimately, whether a particular spot on the nipple surface is or is not a ductal orifice can be determined by whether it can be cannulated or accessed with a guidewire after identification.

The devices for use in an embodiment of the invention can also comprise an ability to image the characteristic electrical signal electronically, or the device can provide for a photograph to be taken of a marked nipple surface. An electronic image, a printed image, and an image as a result of exposure to sensitive film, including e.g., X-ray film can be generated from the device or upon generation of a potential and/or current through the breast by the device.

The electrodes useful for practicing the method using characteristic electrical signal can be made essentially as described in U.S. Pat. No. 5,217,014 to Biofield Corp., and WO 96/12439 (incorporated by reference herein in their entirety) with modifications appropriate to the diminutive size of the nipple surface and the ductal orifices relative to the items being detected in these patents and publications, and also with regard to the reduced conductance per ductal orifice that it is the object to identify; which patents/publications are herein incorporated in their entirety by reference thereto. The test electrodes can be designed as described herein, e.g., a single test electrode moved sequentially to locations on the nipple surface.

One embodiment of the invention includes a kit for identifying and accessing a breast nipple ductal orifice comprising a reference electrode and a test electrode capable of being connected to a machine capable of generating an electrical potential or current in an electrical circuit. The machine capable of generating an electrical potential or current in an electrical circuit can generate a potential between the two electrodes and measure a characteristic electrical signal value at a given location on a nipple surface. The kit further includes an accessing element for accessing the ductal orifice once identified. The accessing element can comprise, e.g., a guidewire, a cannula, a catheter, or a combination thereof. As described further herein, in preferred embodiments, the accessing element is the catheter and comprises the test electrode, such that one elements provides the function of localizing (test electrode), cannulating (accessing element) and then infusing (catheter) the individual milk duct. Accordingly, in one preferred embodiment of the kit, a plurality of catheters (e.g., those shown in FIGS. 8A-D) are provided such that all of the milk ducts in one or two nipples may be cannulated and treated. The number of catheters may vary between about 1 and 12, and more preferably from about 6 to 10.

The machine capable of generating an electrical potential or current in an electrical circuit can be operably connected to a machine for analyzing the electrical signal, (e.g., a central processing unit (CPU)) e.g., comprising a computer program capable of analyzing a plurality of characteristic electrical signal measurements taken from the nipple surface. The machine for analyzing the electrical signal can also be capable of capturing the characteristic electrical signal values as electronic information, storing this information, and imaging it, e.g., either on the computer screen, on a printed piece of paper, in a graph, or other useful image. In some embodiments, the kit can further comprise instructions for use of the kit. In some embodiments, the kit can further comprise an element to mark the duct location on the nipple surface once the orifice is identified, e.g., a marker tube that can be placed within the duct to mark the ductal orifice location. In some embodiments, the kit further comprises an amount of prophylactic and/or therapeutic agent(s) such as those described below, sufficient to infuse all of the target milk ducts. In one preferred embodiment, the CPU/ESU may be disposable. In another preferred embodiment, the device generates an auditory signal when the ductal orifice is localized.

In another preferred embodiment, the characteristic electrical signal is detected as described in detail below with reference to FIGS. 9-17.

### Delivery of Materials to Ducts

In some embodiments, a material such as an imaging agent, a prophylactic or therapeutic agent, or a hydrogel and combinations thereof, can be delivered to a breast duct by identifying and accessing the duct by techniques including devices or methods described herein, and injecting or otherwise instilling the material. In some embodiments, the material can also comprise a carrier, such as for example a liquid and/or a hydrogel that can be delivered in liquid form and which forms a more viscous, semisolid or solid carrier in situ.

Suitable carriers include low viscosity fluids (such as water and water based materials or solutions such as DI water, water for injection, saline, buffered saline, buffered water, etc. as well as other low viscosity fluids or solutions such as those comprising glycerol, oils, partial glycerides, etc.), polymeric based fluids or solutions (such as PEG, substituted PEG, peptide solutions, polysaccharide solutions, etc.), and hydrogel-based materials.

Carriers can be associated with agents (including imaging agents, therapeutic agents and prophylactic agents), as by chemically bonding the agent to one or more components of the carrier with covalent or ionic bonds. Agents can also be associated with one or more components of a carrier by attractive forces such as hydrogen bonding, van der Waals forces, hydrophobic or hydrophillic interactions, etc. Agents can also be physically entrapped by one or more components of a carrier, such as in a gel matrix or with another suitable entrapment system.

In some embodiments, a carrier can include a compound that has sufficient size, surface charge, and hydrophillic/hydrophobic characteristics to limit its passage through the ductal epithelium. In some embodiments, the passage limiting compound can be associated with an agent, such as by covalent linkage, ionic linkage, or sufficient hydrogen bonding, van der Waals interaction, or physical entrapment to serve to inhibit the passage of the agent into the ductal epithelium.

In some embodiments, carriers can include PEG scaffolds, such as PEG having multiple thiol or thiol reacting groups as well as copolymers of PEG and compounds having thiol groups can be used as scaffolds, or precursors/intermediates for hydrogels, agent delivery systems, nanogel particles, and aggregated nanogel particles. PEG scaffolds include polymers containing PEG-thiol groups, polymers containing PEG and peptide thiol groups, copolymers of PEG and compounds containing thiol groups, and materials including PEG and thiol-reactive groups.

PEG scaffolds, in various embodiments, can be complexed with one or more agents, such as a therapeutic or imaging agents, can be crosslinked with itself or another compound, crosslinked and aggregated, crosslinked and complexed with an agent, or crosslinked and aggregated and complexed with an agent.

Suitable PEG scaffolds include PEG polymers, block polymers, block copolymers and copolymers described below:

PEG Polymers with Thiol Groups—

Materials containing PEG polymer with multiple thiol terminus groups can serve as PEG scaffolds. Suitable materials include PEG having a molecular weight in the range of about 1,000 to about 100,000 Da, with more than 2 thiol groups. Multi-arm PEG and branched PEG are suitable as scaffolds, including multi-arm PEG having 2-, 3,-4-, or 8-arms, where two or more or even all of the arms have a thiol group. In some embodiments, the thiol group will be unbound, and available for replacement of the hydrogen with another group. In other embodiments, the thiol group can have a different group bound to it that is replaced with a desirable group during complexation, crosslinking, or some combination of the two.

In one preferred embodiment, a multi-arm thiol-terminated PEG nanocarrier such as the 8-arm thiol PEG shown below can be used as a scaffold. The central portion can include central junction with PEG moieties, linked to the central portion with ether linkages, or other suitable linkages, and terminating in a thiol group for at least some of the PEG moieties.

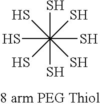

In some embodiments, multiple thiol groups can be achieved by branching a linear PEG, or by branching a multi-arm PEG, and terminating at least a portion of the branch PEG units with a thiol group. Through branching or a combination of branching and use of multi-arm PEG, the number of thiol groups desired, such as 2, 3, 4, 8, or more can be achieved.

PEG with Peptide Thiol Groups—

The PEG scaffold can include PEG polymer containing multiple units of peptide thiol groups, such as by incorporating multiple cysteine moieties into the structure, either together or separated. Some embodiments can have polycysteine sections incorporated into the structure of the scaffold. Suitable scaffolds can have a molecular weight in the range of about 1,000 to about 100,000 Da.

Copolymer Containing Thiol Groups—

The PEG scaffold can include copolymers of PEG and thiol-containing compounds, such as mercaptosuccinic acid as well as polymers and derivatives of mercaptosuccinic acid. The PEG portion of the copolymer can be functionalized to have thiol or thiol-reactive groups, or not. When the PEG portion is functionalized, the functionalization can occur prior to or after copolymerization. Other suitable copolymerization compounds include peptides having multiple thiol groups, such as contributed by cysteine moieties or provided by functionalizing an amino acid moiety to have a thiol group, or other suitable copolymerization compounds having functional groups suitable for copolymerization with a PEG or functionalized PEG, and providing multiple thiol groups between the PEG portions in the final copolymer.

PEG with Multiple Thiol Reactive Groups—

The PEG scaffold can include PEG with thiol reactive groups which form thiol ether or disulfide bonds when used in combination with, or in place of the thiol groups, as described above. When a thiol reactive group is utilized on the PEG scaffold, a compound used to attach an agent or to crosslink will generally utilize a thiol group or other suitable nucleophilic group to form a thiol ether or disulfide bond with the scaffold. Suitable thiol reactive groups include those that can be used in conjunction with a thiol-containing compound to attach an agent, or that can react with a thiol group to achieve crosslinking of the PEG scaffold or PEG scaffold with agent. Suitable thiol reactive groups include for example maleimide and vinylsulfone. PEG having multiple PEG reactive groups, where the groups are all the same, all different, or a mixture can also be utilized.

Other PEG hydrogels such as those described in U.S. Pat. Nos. 5,874,500, 7,176,256 and 7,151,135 can also be used in embodiments of the invention; these patents are incorporated herein in their entireties by reference. Any substances or combinations that can be infused in liquid form and which gel or otherwise react to form more viscous, semisolid or solid gel-like materials in situ may be used in accordance with certain embodiments of the invention.

In some embodiments, carriers can include PEG nanocarriers, such as those formed by complexing a PEG scaffold with a suitable agent. In some embodiments, the complexation is by covalent bond, such as by the formation of a thioether or disulfide bond with the agent or an intermediate, in some embodiments, the complexation can be by non-covalent techniques, such as by ionic bonding, hydrogen bonding, hydrophobic/hydrophillic interaction, van der waals interaction, physical entrapment, etc. Suitable agents include therapeutic agents, such as drugs or other compounds/materials used in the treatment of disease; diagnostic agents, such as those compounds/materials used to identify or evaluate medical conditions or diseases; and imaging agents, such as compounds/materials used in imaging of biological features, such as visually, microscopically, radiographically, electronically, sonographically, photographically etc.

In some embodiments, the PEG scaffold can have more than one agent molecule complexed to a PEG scaffold molecule. In some embodiments, there can be 2 or 3 or 4 or 5 or 6 or 7 or 8 or more agent molecules complexed to a PEG scaffold molecule. In some embodiments, the agent can be attached directly, such as through a covalent bond to the PEG scaffold. Suitable linkages include for example thioether and disulfide linkages. In some embodiments, the agent can be attached to the PEG scaffold through a linking compound, where the linking compound is bonded to both the PEG scaffold and the agent. Suitable linking compounds include for example peptides and compounds that have a peptide portion, such as an Arg-Gly-Asp-Cys sequence, a Leu-Gly sequence, or a Glu(Leu-Gly)₂ sequence.

In some embodiments, the attachment of the agent can favorably modify the functionality of the agent, such as by modifying the solubility, the time release, the stability, the bioavailability, or the targeting of the agent. Some agents, on their own, have only limited solubility in biologically relevant solvents, such as water, water for injection, saline, or buffered saline. Attachment of the agent to PEG scaffolds to form a nanocarrier can, in some embodiments, result in a higher solubility of the nanocarrier than for the agent alone on a gram/milliliter basis. In some embodiments, the increase in solubility may be sufficient to overcome the increase in molecular weight of the nanocarrier as compared to the agent, and provide a higher soluble dose. When more than one molecule or portion of agent is attached to a nanocarrier, higher agent dosing can be achieved with the same number of grams of nanocarrier as compared to a nanocarrier with only one molecule or portion of agent attached. However, as the amount of agent attached to a nanocarrier increases, the characteristics of the nanocarrier would generally be expected to become more like those of the agent itself, resulting in, for example, a decrease in concentration that can approach the solubility of the agent itself.

In some embodiments, such as in the use of a multi-arm PEG attached to agents having low solubility, such as an agent that is sparingly or slightly soluble, attachment of additional agents to the PEG scaffold results in only a limited effect on the solubility of the nanocarrier achieving adequate water solubility and, at the same time, a therapeutically relevant drug dose. In addition, catepsin B sensitivity and bioadhesive targeting can be combined by selection of additional groups to attach to the PEG scaffold, such as the use of Leu-Gly or Arg-Gly-Asp as a linker for the agent to the PEG scaffold.

In some embodiments, a carrier can be a PEG with functionality other than a thiol or a thiol-reactive group, which is suitable for PEGylation of an agent to the PEG backbone. Suitable linkages include those utilizing an ester, amide, ether, etc. linkage.

In some embodiments, a targeting capability can be incorporated into a carrier or an agents. A molecule or molecule with a subsection that selectively adsorbs or reacts with particular cells or moieties on the surface of particular cells can be incorporated into a carrier and associated with an agent to increase the localization of the delivery of the agent. In some embodiments, the targeting composition can be administered to a duct, and then drained or rinsed from the duct, resulting in an elevated concentration in the vicinity of the particular cells of interest.

### Imaging Agents

Imaging agents of various kinds can be provided to a duct in order to modify visual or machine imaging, such as to improve contrast; highlight particular features, such as diseased tissue, or disease precursor tissue; identify ductal networks, etc. In various embodiments, the imaging agent can absorb electromagnetic radiation of particular wavelengths, emit electromagnetic radiation of particular wavelengths, cause a change in the index of refraction, polarization, or other measurable characteristic of electromagnetic radiation or sound. Electromagnetic radiation that is absorbed, reflected, emitted or otherwise modified can include electromagnetic radiation in the visible, UV, IR, near IR, x-ray, gamma radiation, as well as other portions of the spectrum, whether visible or not, as well as combinations of different portions of the spectrum.

In some embodiments, an imaging agent can be used to improve imaging during sonic or ultrasonic examination, such as during an ultrasound, and can include the introduction of bubbles or microbubbles, or the generation in situ of bubbles/microbubbles. Bubbles or microbubbles can be in the form of solid, hollow, gas filled, or liquid filled spheres or other shaped objects. In some embodiments, ultrasound imaging agents can comprise shells that are rigid, semirigid or soft, and can comprise polybutyl-2-cyanoacrylate. In some embodiments, an imaging agent can improve imaging during radiographic examination. In some embodiments, an imaging agent can improve imaging during MRI, SPEC, PET, or scintigraphy examination.

In some embodiments, the imaging agent can be a bioreactive compound, such as one that reacts in-vivo to become more easily detected or less easily detected. Bioreactive compounds can provide improved imaging of particular anatomic features through the change of imaging quality that occurs in the vicinity or away from the vicinity of the anatomical feature being examined. In some embodiments, the bioreactive compound can react with a cell or a feature of a cell, such as a surface protein, etc. or with a compound secreted by a cell. In some embodiments, the bioreactive compound can react with a compound that is administered.

Suitable agent include, but are not limited to, dyes such as toluidine blue, methylene blue, or indocyanine green; X-ray attenuating materials such as those comprising barium, barium sulphate, iodine gastrograffin, or other suitable materials; materials having magnetic properties, such as diamagnetic, superparamagnetic or ferromagnetic, including those comprising oxygen, nitroxides, and ions of iron, magnesium, gadolinium, or dysprosium, such as gadolinium chelates, GD-DTPA, Gd-DTPA-BMA or GD-DOTA, etc.; technitium compounds, such as Tc-99; iodine compounds, such as triiodinated benzoic acid, diatrizoate, iothalamate, ioversol, iohexyl, iopamidol, etc.; Indium-111; Fluorine-18 compounds, such as fluorodeoxyglucose; 0-15; C-11; N-13; etc.

Agents can also include fluorescing compounds or fluorescing groups attached to other compounds. Suitable fluorescing compounds or groups include, for example, tris(4,4-diphenyl-2,2-bipyridine)ruthenium(II) chloride, tetramesitylporphyrin, tetra-t-butylazaporphine, nile blue, tris(2,2-bipyridyl)ruthenium(II) chloride, as well as other compounds and groups well known in the art, see, for example Fluorophores.org (lamp3.tugraz.at/˜fluorbase/ and lamp3.tugraz.at/˜fluorbase/BrowseSubstance.php, last viewed Apr. 15, 2009). Fluorescing compounds and groups can be associated with carriers or other compounds described herein, through covalent bonding, ionic bonding, hydrogen bonding, van der Waals association, physical entrapment, or other means as appropriate.

In some embodiments, the use of agents having higher toxicity than those normally used is possible due to the limited exposure due to localized delivery as well as the optional use of hydrogels or viscous materials which can limit the uptake or interaction of the agent with the body.

### Persistence of Agents and Hydrogels

In some embodiments, the agent, the carrier, or a combination of agent and carrier can provide a sustained presence of the agent, the carrier, or both within a duct. In some embodiments, sustained presence can be utilized to achieve a time release characteristic, such as for therapeutic or prophylactic agents. Sustained presence of agents can limit the rate of uptake of agents by the body resulting in a limitation in systemic activity of the agent while maintaining a relatively high local activity, which in some embodiments can be utilized to achieve local treatment of, for example, cancerous or precancerous cells including cancerous or precancerous ductal epithelial cells.

In some embodiments, a sustained presence can provide, for example, opportunity of follow-up examination hours, days, weeks, months, or longer after the initial introduction of the material to the breast. In some embodiments, an imaging agent with a carrier that has sustained presence can be introduced to the duct to create a ductal map prior to surgery where the ductal network is more visible than without the imaging agent and carrier. During surgery, the ductal map allows for improved surgical procedure by facilitating identification of the entire ductal network and by facilitating the identification of the tissue for removal. Sustained presence of the ductal map can allow evaluation days, weeks, months, or longer after the surgical procedure to determine if the entire ductal network was removed.

In some embodiments, a hydrogel can be formed in or administered in a duct that has extended persistence, such as by utilizing a PEG thiol crosslinked by HBVS. In some embodiments, a less persistent hydrogel can be formed or administered in a duct by utilizing PEG or derivitized PEG crosslinked by other crosslinking agents or by utilizing a different hydrogel formation system, such as one based on polysaccharides or peptides, such as ionically crosslinked polysaccharides or peptides, including alginates, gelan, pectin, carageenan, cellulosic, gelatin, collagen, hyaluronic acid, etc.

Persistence or sustained presence/sustained release can be achieved, for example by selection of the association system between an agent and the carrier. Generally, agents covalently linked to a carrier will be more persistent and/or have slower release, and/or have more local effect than those ionically bonded or associated by other techniques. Selection of other characteristics of carriers and agents can also increase or decrease the persistence of an agent, such as by selecting a crosslinked carrier that is itself more persistent. Other parameters that can affect the persistence of an agent include the relative hydrophillicity/hydrophobicity of the carrier and agent, the lability of the linkage between the agent and carrier, the concentration of the carrier (with higher carrier concentrations frequently resulting in greater persistence), and the biodegradability of the carrier or the agent itself.

### Breast Augmentation and Asthetic Applications

In the application of intraductal therapy to breast re-constructive or non-reconstructive augmentation or cosmesis, a variable amount of biomaterial may be injected into a varying number of individual ducts in the breast to achieve a variable volume and shape increase in the breast. The property of preferred gel compositions are that they are low viscosocity on infusion, but following a period of time, become more viscous, and then a solid material or a hydrogel. By using a biodegradable material, it may be demonstrated that the cycle of repeated infusion and degradation of the biomaterial within a duct will allow for a greater volume of material to be infused into the duct over time.

Hydrogels may have different physical mechanical and biochemical properties. The time for the components of a hydrogel in solutions to form the final material after mixing of solutions may be variable. Most commonly for biomedical applications, the set time is very short, resulting in rapid polymerization and a near solid material. However, in order to provide for extensive intraductal delivery, the set time must be longer. It has been demonstrated that the set time of polymers may by modified so that a 10 min or longer set time is achieved. Factors such as percentage volume loading of components for mixing, presence of interfering compounds, and number of molecular attachment points (# of crosslink's) may be used to change the set time of polymers.

The durability of the hydrogel may also be varied. Hydrogels may vary from nearly permanent, to rapidly degraded. The bond type and number of molecular attachment points may be varied to achieve hydrogels with different durability. The inclusion of interfering molecules may also alter the durability of the biodegradable polymer. It has been demonstrated that an activator with PEG results in a very long term stable hydrogel. Alternatively, using the same PEG, but selecting for a different bond type may shorten the durability of the hydrogel, as demonstrated by the S—S bond crosslink or the amide bond type crosslinked PEG polymer.

The material properties of the material may also be modified by changing the number of intramolecular crosslinks. For example 4-arm and 8-arm PEG may be commercially obtained, and used to create materials with different mechanical properties depending on the number of crosslinks made and the number of free arms.

Alternatively, a blend of materials may be made used that in isolation would have a very rapid set time, and a very slow set time that achieve an intermediate set time. Likewise a blend of materials may be used that have more and less rigid components before blending.

The type of materials that may be used for this application include alginate, PEG, and any of a number of other biopolymers which are capable of forming a solid or hydrogel.

The ability of the material to form a softer hydrogel instead of a more rigid material after setting, may provide for a decreased risk of perforation of the duct.

In some embodiments, suitable gel materials may include those capable of forming a viscous or otherwise relatively non-flowable material, including materials having shear thinning, thixotropic, plastic, bingham plastic, Newtonian, or not Newtonian characteristics serving to limit or reduce outflow from a duct. These materials may or may not exhibit a yield point where flow or cracking of the new gel matrix can occur only after a particular value of shear rate or stress is applied.

Some preferred materials include those having a polymeric component, including, but not limited to, polysaccharides, polypeptides, polyethers, and derivatives thereof. Frequently, the individual polymeric strands can interact with themselves and other strands through van der Waals, ionic, or covalent linkages, either directly or through additional chemical compounds. Preferred polysaccharides include alginates, gelan gum (deacylated, high acetyl, low acetyl, high glyceryl, low glyceryl, intermediate levels of acetyls and/or glyceryl, and mixtures thereof), cellulosics, pectins, cellulose (including microcrystalline cellulose, and bacterial cellulose), agar, fucoidan, chitan, chitosan, carageenans, hyaluronic acid, propylene glycol alginate: polysaccharides containing glucose, fructose, galactose, guluronate, mannuronate, 6-carbon sugars, acids of 6-carbon sugars, 5-carbon sugars, and acids of 5-carbon sugars; etc.

Polypeptides that may be useful include those having high proline content, such as gelatin and materials related to or derived from gelatin and/or collagen.

Embodiments of the gel composition include those which exhibit an increase in viscosity or reduced ability to flow once the material is placed in the duct or in the breast region, but selection of an appropriate rheological profile of the material for the dimensions of the duct or characteristics of the region for placement can also be employed.

Gelation mechanisms, or mechanisms for increasing the viscosity or reducing flow tendency can include use of monovalent ions, multivalent ions, acids, bases, or covalent reagents (including reagents such as glutaraldehyde or other carbonyl or polycarbonyl compounds) combined with the polymeric material or polymer solution prior to introduction to the breast or after introduction to the breast. In some embodiments, a slow release or time release aid can be used in conjunction with a gelling agent, such as glucono delta lactone. In some embodiments, the material may exhibit gelation, an increase in viscosity or a reduction in flow tendency upon heating or cooling. In such embodiments, a method of employing the material is to introduce a material that has a greater flow tendency at a temperature above or below body temperature, introduce the material while in an easier flowing state, and then to allow the material to thermally equilibrate with the body temperature. In other embodiments, the material may be introduced and then heated or cooled (with external heating/cooling or by other means, such as with an implantable device, such as a catheter or other suitable device) to effect gelling or viscosification.

Gel removal can be accomplished, for example through normal bodily functions, such as those that breakdown the material, or through the introduction of a material, either at the time of implanting the gel material or at another time, of a material that will reduce the viscosity, increase the flow tendency, or breakdown the material itself. Suitable materials include oxidizing agents, depolymerizing agents, acids, bases, enzymes, and the like. Preferred materials include enzymes such as proteases or polysaccharases; combinations of fatty acid-containing material, especially those having unsaturated fatty acids, and lipoxygenase or other suitable enzyme, where the fatty acid material can be present in the gel material when placed, or added later; or hydrogen peroxide or in situ generators of hydrogen peroxide. Other techniques for removal of the gel material can include heating or dilution of the material. In some embodiments, two or more of these techniques can be combined.

Some preferred gel compositions include those hydrogels disclosed by Sinko et al., in WO 2006/069344 and WO 2008/133918, which disclose controlled release hydrogel compositions for administration of therapeutic agents to a mammal, comprising a cross-linked polymer matrix; these publications are incorporated herein in their entirety by reference thereto. Although most of the hydrogel disclosures disclosed herein are directed to a controlled release platform for drug delivery, the polymers may be used in accordance with preferred aspects of the present invention without any drug delivery component. On the other hand, in some embodiments, the breast augmentation gel composition may contain an active agent, for example to provide prophylactic or therapeutic treatment of breast cancer (see, e.g., Sukumar, US 2006/0292113 A1; incorporated herein in its entirety by reference).

Hydrogels are three dimensional, hydrophilic, polymeric networks which swell in water without dissolving and retain large quantities of water. They typically comprise both natural polymers such as starches and cellulose derivatives and synthetic polymers and copolymers such as polyethylene glycol and poly(glutamic acid). The networks within the hydrogel can be crosslinked either chemically or physically by cohesive bonds such as ionic interaction, hydrogen bonding or hydrophobic interactions. Alginates and chitosans are examples of polymers that are cross-linked by ionic interactions. Hydrogels are suitable for numerous applications such as implants, contact lenses, membranes for isosensors and drug delivery devices.

Suitable hydrogels for use in accordance with aspects of the present invention can be formed from synthetic polymers such as polyethylene glycol, polyethylene oxide, polyvinyl alcohol, polyvinyl pyrrolidone, polyacrylates, poly(ethylene terephthalate), poly(vinyl acetate), and copolymers and blends thereof, as well as natural polymers such as cellulose and alginate.

Preferred polymers comprise at least two thiol groups, and may be a homopolymer or a copolymer.

The hydrogels include at least one cross-linkable polymer which may be cross-linked to entrap an encapsulated therapeutic agent. Any cross-linkable polymer, which bears two or more functional or reactive groups capable of participating in a cross-linking reaction to form a matrix of the invention may be used. Such functional groups include but are not limited to amino, carboxyl, thiol and hydroxyl groups, or combinations thereof; reactive groups include vinylsulfone, maleimide, pyridyldithio, and other moieties capable of reacting with the aforementioned functional groups, among others. A preferred polymer is one on which at least two thiol groups are present and is cross-linked with a thiol-reactive bifunctional cross-linking reagent in the presence of the encapsulated therapeutic agent.

Selection of the appropriate polymer, the concentration in the matrix, the extent of functional groups capable of participating in cross-linking, the type of cross-linking agent, and the extent of cross-linking, and other factors may be governed by the desired controlled release properties of the composition.

Examples of suitable polymers for the preparation of the polymer on which at least two thiol groups are present include both homopolymers or copolymers. By way of non-limiting example, suitable polymers, which may be chemically modified to comprise thiol groups, include polyalkylene oxides such as poly(ethylene glycol) &lsqb; also known as polyethylene glycol or PEG, polyethylene oxide or PEO&rsqb; carboxymethylcellulose, dextran, polyvinyl alcohol, N-(2-hydroxypropyl)methacrylamide, polyvinyl pyrrolidone, poly-1,3-dioxolane, poly-1,3,6-trioxane, polypropylene oxide, a copolymer of ethylene/maleic anhydride, a polylactide/polyglycolide copolymer, a polyaminoacid, a copolymer of poly(ethylene glycol) and an amino acid, or a polypropylene oxide/ethylene oxide copolymer. Such polymers are then derivatized or further polymerized to introduce thiol groups; chemical modification of the polymer may be necessary as a step prior to the further derivatization to incorporate thiol groups. In certain embodiments, for example, a polymer of the present invention may be derived from a poly(ethylene glycol) (PEG) derivative, for example, cz,o-dihydroxy-PEG or a,a-diamino-PEG, but other derivatives are embraced herein. In certain embodiments the polymer comprising thiol groups may be, for example, a polymer of a,n-diamino-poly(ethylene glycol) and thiomalic acid; a polymer of a,co-dihdropoly(ethylene glycol) and thiomalic acid; or a polymer of a,w-dicarboxy-PEG subunits and lysine wherein the free carboxy groups on the lysine residues are derivatized to form thiol groups. These polymers are only examples of possible choices, as the skilled artisan will be aware of numerous alternatives. The selection of the polymer, or combinations thereof, may be guided by the desired properties of the final product such as, for example, the duration of release the therapeutic agent and the release kinetics. In certain embodiments, a product of the invention may comprise more than one polymer component in order to provide two or more different release characteristics. In certain embodiments, more than one therapeutic agent may be included.

In certain preferred embodiments, a polymer of the present invention is derived from a poly(ethylene glycol) (PEG) derivative, for example, a,co-dihydroxy-PEG or a,w-diamino-PEG, but other derivatives are embraced herein. Examples of such polymers with particular molecular weights include a co-dihydroxy-PEG3,-400; a,w-dihydroxy-PEG1,ooo; a,co-diamino-PEG.-3,400; and a,a)-diamino-PEG1,ooo. PEG is known to be a particularly nontoxic polymer. These derivatized PEG subunit polymers may be used as amino- and hydroxy-containing polymers for cross-linking, or may be further derivatized, for example, to prepare the polymer on which at least two thiol groups are present by derivatization with thiomalic acid. Thiomalic acid (also known as mercaptosuccinic acid) may be replaced by dimercaptosuccinic acid, thereby doubling the number of sites available for cross-linking. Increasing the extent of cross-linking the matrix results in a gel with smaller pores.

In certain embodiments, the formulations of the invention are prepared from a mixture of polymer matrix and liposomes which comprises at least one therapeutic agent, preferably the polymer matrix including a polymer capable of being cross-linked, and forming cross-links between the polymer molecules to form a cross-linked matrix entrapping the liposomes included the therapeutic agent. In certain embodiments, the cross-linking can be performed before, during, or after the matrix is administered to a mammal. For example, the cross-linking reaction can be initiated in vitro, and the mixture, while undergoing cross-linking, may be injected into a bodily compartment of a mammal, wherein the injected bolus continues to cross-link and harden in situ. In certain embodiments, a cross-linked matrix after formation can be implanted or inserted into the location of the body from which delivery of the agent is desired. The compositions may also be introduced at either end of the gastrointestinal tract for transmucosal absorption.

In certain embodiments, the polymer moieties may be cross-linked by reagents capable of forming covalent bonds between the functional groups, such as but not limited to homobifunctional and heterobifunctional cross-linking agents. As described above, a preferred moiety is a thiol group. In certain embodiments, a preferred cross-linking agent is one that forms thioether bonds, such as a vinylsulfone or maleimide, but the invention is not so limiting. Other cross-linking reagents, such as a pyridyldithio-containing reagent, or oxidation, maybe used to generate reducible cross-links. Combinations of cross-linking reagents maybe used, to provide a ratio of cross-link types which generate the desired release characteristics of the composition. In certain embodiments, the preferred thiol-containing polymer has from 2 to about 20 thiol groups, preferably from about 3 to about 20 thiol groups, and most preferably from about 3 to about 8 thiol groups. In certain embodiments, the thiol groups on the polymer are sterically hindered.

Various conditions and/or reagents may be used to effect the cross-linking of the polymer, depending on the particular functional groups on the polymer. By way of non-limiting example, the conditions that cause cross-linking of the thiol groups on a thiol-containing polymer may be reaction in the presence of an oxidizing agent or reaction with a cross-linking agent. In the aspect of oxidation, the oxidizing agent may be by way of non-limiting example, molecular oxygen, hydrogen peroxide, dimethylsulfoxide, and molecular iodine. In the aspect where a cross-linking agent is used, the cross-linking agent may be a bifunctional disulfide-forming cross-linking agent or a bifunctional thioether-forming cross-linking agent. In a preferred embodiment, the cross-linking agent is a long-chain cross-linking agent, with a molecular weight of about 300 to about 5,000 Da. Non-limiting examples of suitable cross-linking agent include 1,4-di-&1sqb;3 t,2′-pyridyldithiopropionamido)butane&rsqb; a,co-di-O-pyridyldisulfidyl-poly(ethylene glycol); a vinyl sulfone such as a ci-divinylsulfone-poly(ethylene glycol); 1,11-bis-maleimidotetraethylene glycol; and a,co-diiodoacetamide-poly(ethylene glycol).

For other functional groups or a combination of a thiol group and another group, any appropriate bifunctional cross-linking agent may be selected which will achieve the desired cross-linking of the functional groups aid formation of the cross-linked polymer.

In another aspect, the polymer additionally comprises a functional group, which may derivatized for example with a label, such as a contrast/imaging agent, radionuclide, chromophore, fluorophore, red or near-infrared fluorophore, or nonradioactive isotope. In certain embodiments, the label is a metabolically stable polymer component that after piymer is detectable in the urine. In another embodiment, the cross-linking agent used to cross-link the polymer additionally comprises a functional group, such as a label.

In certain embodiments, the release rate degradation of the hydrogel may be regulated by the biodegradability of the cross-linked polymer matrix, the liposome or combination thereof. In certain embodiments, the degradation rate may be adjusted by varying the ratio or types of cross-links of the matrix, and the stability or lability thereof, in the composition. For example, preferably, the ratio of reducing agent-sensitive disulfide bonds, esterase-sensitive ester bonds, and stable thioether bonds may be selected to provide the desired release kinetics of one or more entrapped liposomes. In certain embodiments, the release rate is adjusted by adjusting the pore size of the pores in the hydrogel matrix which allows for the release of the liposome from the matrix. For example, compounds or liposomes with molecular weights greater than the pore size of the hydrogel matrix will be trapped within the hydrogel and release slowly. In preferred embodiments, the concentration of the copolymer the type and amount of cross-linker and the solution pH can be manipulated to form hydrogels of varying pore size.

In preferred embodiments, the gel composition may be selected to provide certain functional characteristics. For example, the rate of absorption may be varied by adjusting the composition of the gel. Thus, a patient may choose to have a breast augmentation procedure which lasts for a relatively short duration, e.g., 12-72 hours, for a party, anniversary or other special occasion. Longer durations, e.g., 1, 2, 3, 4, 6-12 weeks, or 4, 5, 6, 7, 8, 9, 10, 11 or 12-16 months, can be provided by gel compositions that degrade gradually. This temporary and adjustable nature of the augmentation through ductal administration of a gel composition provides great market advantages.

Besides flexible and customizable duration times, another advantage over surgical implants, is that the size of the breast can be increased gradually, so patients and their family, coworkers, and other acquaintances can grow accustomed to the size change, perhaps without notice, and certainly without noticing a dramatic and/or embarrassing increase in breast size.

Another advantage of the present augmentation through ductal administration of a gel composition over surgical implants is that the shape of the breast can be altered by administering different amounts of gel to different ducts. The location of the ductal orifice on the nipple is generally related to the location of the ductal system accessible via that orifice. Thus, for example an orifice identified on the left side of the nipple would be expected to open onto a milk duct that is oriented on the left side of the breast, whereas an orifice on the lower/bottom side of the nipple would be expected to open onto a milk duct that is oriented on the lower/bottom side of the breast, etc. Accordingly, one side (left, right, top, bottom, etc.) may be augmented to a greater degree than another side, in order to correct natural asymmetries in breast appearance. Lower ducts may be augmented to provide greater lift.

A kit for breast augmentation is disclosed in accordance with certain embodiments. The kit may comprise an analyzer configured to indicate a characteristic electrical value in an electrical circuit comprising a duct of the mammary gland; a catheter operable to access the duct when the analyzer indicates the characteristic electrical value; and a gel composition capable of introduction through the catheter and effective to swell the treated milk ducts. The analyzer may comprise one or more of a processing unit, a conductor, and an indicator for indicating when the characteristic electrical value has been provided. The catheter may comprise a distal duct probe for detecting the characteristic electrical value. In further embodiments, the catheter may be sized so as to be accommodated within a marker tube, which itself is sized to access the duct. In further embodiments, the kit may also comprise a guidewire, which may be fixed with respect to the catheter or capable of motion relative to the catheter. In other embodiments, the gel composition may comprise one or more biocompatible and biodegradable polymeric materials. In some variations, the kit may comprise several different vials containing different gel compositions, so that the physician or medical personnel administering the augmentation procedure can vary the functional characteristics based on the desired aesthetic effect.

In a further embodiment, a kit for breast augmentation is provided that comprises: a duct accessing device comprising: a pair of electrodes, at least one of which is configured to establish electrical communication with the nipple surface; a catheter sized to access the mammary gland; and a gel composition capable of introduction through the catheter and effective to augment the breast size and/or shape. The electrodes may include a test electrode that is placed at putative sites of mammary gland ducts, and a reference electrode that is placed elsewhere on the body, such as at the base of the breast. The electrodes may function to detect a characteristic electrical value when the test electrode is placed at the site of a mammary gland duct. The characteristic electrical value may be one of a capacitance value, and impedance value, a reactance value, or any other value known in the art. In embodiments, the catheter may be sized so as to be accommodated within a marker tube, which itself is sized to access the duct. In further embodiments, the kit may also comprise a guidewire, which may be fixed with respect to the catheter or capable of motion relative to the catheter.

Other kit embodiments contemplate the use of a marker dye, comprising: a nontoxic marker dye or ink that indicates the location of ductal orifices when injected into ducts of the breast; a catheter operable to access the indicated ductal orifices; and a gel composition capable of introduction through the catheter and effective to augment the size and/or shape of the breast. The dye or ink may be toluidine blue, methylene blue, indocyanine green, or any other dye known to be useful for the purpose temporarily or permanently marking the body. In further embodiments, the catheter may be sized so as to be accommodated within a marker tube, which itself is sized to access the duct. In further embodiments, the kit may also comprise a guidewire, which may be fixed with respect to the catheter or capable of motion relative to the catheter.

Embodiments of the methods include a method of breast augmentation, comprising: providing a catheter having an atraumatic tip and a lumen; locating multiple ductal orifices on a nipple using at least one of a duct locating instrument and a compound that visually enhances the location of the ducts; cannulating at least a first ductal orifice by inserting the atraumatic tip into the first orifice; and delivering an agent through the lumen and into the first orifice. The duct locating instrument may comprise one or more of the atraumatic tip, a processing unit, a conductor, and an indicator for indicating when the instrument is located at a duct. In other embodiments, the duct locating instrument may comprise one or more electrodes. The compound may comprise a dye such as toluidine blue, methylene blue, or indocyanine green. In an embodiment, the compound may be delivered by injection into the ducts. In other embodiments, the atraumatic tip may comprise a solder ball or a coil at the distal end of the catheter. In further embodiments, the catheter may be sized so as to be accommodated within a marker tube, which itself is sized to access the duct, and after insertion of the atraumatic tip into the ductal orifice, the marker tube may be advanced over the catheter and into the ductal orifice. Following delivery of the gel composition, the atraumatic tip may be removed from the ductal orifice, leaving the marker tube in place within the orifice.

In further embodiments, the method includes cannulating a second ductal orifice using the same catheter, and delivering the same or different gel composition into the second orifice. In another embodiment, multiple catheters are used to cannulate the multiple ductal orifices, followed by the delivery of the gel composition(s) to the ducts. In other embodiments the multiple catheters are used to cannulate all of the required ducts for the sake of convenience or to avoid the need of using markers. In other words, the methods contemplated include both methods in which a single catheter is used to access multiple orifices for gel delivery, and methods in which a new catheter is used for each ductal orifice that is accessed.

In another embodiment, a method is provided for breast augmentation, wherein the method comprises the steps of providing an apparatus for locating and cannulating a ductal orifice, the apparatus comprising a catheter having a distal tip and a lumen, the distal tip comprising a duct probe for locating the ductal orifice; and subsequently cannulating the ductal orifice by inserting the distal tip into the orifice; and administering locally to the duct a gel composition with or without a therapeutic or prophylactic agent. Other agents, such as vitamins, hormones, nutritional supplements, herbal extracts, etc. may also be included in the gel composition, which may promote health of the treated breast. In other embodiments, the duct probe comprises at least one electrode. Further embodiments of the method include both methods in which a single catheter is used to locate and cannulate multiple orifices, and methods in which a new catheter is used for each ductal orifice that is accessed, as described above. Further embodiments include repeating the aesthetic, therapeutic and/or prophylactic procedure at selected time intervals, e.g., a single treatment regimen could include a single treatment or treatments spaced days, weeks or months apart and this regimen could be done once or repeated once a month, once every 3 months, once every 6 months, once a year and/or once every 2-10 years, depending among other indices or the presence of marker antigens, genes, clinical state of the patient and the risk/benefit analysis with respect to development of breast cancer.

### Prophylactic and Therapeutic Agents

Once a duct has been identified and accessed using the devices described above, it is then possible to introduce an agent into the accessed duct via the device in order to prevent or treat diseases arising in the epithelial lining of the duct, as described below. It is also noted that intraductal delivery can be used to target non-epithelial cells within the breast, as the epithelial lining is selectively permeable. Thus, depending on the agent introduced, it may permeate to other cells, e.g., stromal cells. Targeting carriers can be used to fine tune the selective delivery.

Prophylactic Method—

The prophylactic method is a method of treating the ductal epithelium (and/or non-epithelial cells) of an exocrine gland to prevent initiation and/or progression of a disease that affects the exocrine gland. The method comprises contacting the ductal epithelium of the exocrine gland with an agent so as to destroy ductal epithelial cells and/or non-epithelial cells affected by the disease.

In one embodiment of the prophylactic method, epithelial cells along the ductal epithelium of a mammary gland are treated to prevent initiation and/or progression of an abnormal growth profile or morphology, e.g., hyperplastic, transformed, neoplastic etc., growth that may characterize precancerous and/or cancerous phenotypes, so as to inhibit the development of breast cancer of ductal epithelial origin. The method comprises locally contacting, by ductal cannulation or catheterization described above, the ductal epithelium of the mammary gland with an agent selected to destroy at least a portion of the epithelial cells along the ductal epithelium. The agent preferably comprises a cytotoxin (e.g., a conventional chemotherapy agent or combination of agents well known to those of skill in the art), such as for example, genistein, okadaic acid, 1-β-D-arabinofuranosyl-cytosine, arabinofuranosyl-5-aza-cytosine, cisplatin, carboplatin (including in one embodiment, a microencapsulated carboplatin by hydroxypropyl alpha-cyclodextrin), actinomycin D, asparaginase, bis-chloro-ethyl-nitroso-urea, bleomycin, chlorambucil, cyclohexyl-chloro-ethyl-nitroso-urea, cytosine arabinoside, daunomycin, etoposide, hydroxyurea, melphalan, mercaptopurine, mitomycin C, nitrogen mustard, procarbazine, teniposide, thioguanine, thiotepa, vincristine, 5-fluorouracil, 5-fluorocytosine, adriamycin, cyclophosphamide, methotrexate, vinblastine, doxorubicin, leucovorin, taxol, anti-estrogen agents such as tamoxifen, intracellular antibodies against oncogenes, the flavonol quercetin, Guan-mu-tong extract, retinoids, nontoxic retinoid analogs, and monoterpenes. In preferred embodiments, the cytotoxic agent comprises carboplatin or pegylated lipososmal doxorubicin. Alternatively, the agent may comprise a vector comprising a thymidine kinase gene, such as a Herpes simplex thymidine kinase gene, and ganciclovir, a vector comprising a HPRT gene and HAT nucleotide, a cytolytic virus, such as a Vaccinia virus, or ethanol. The method can additionally comprise contacting the ductal epithelium with a cytokine or hematopoietic growth factor, such as GM-CSF. Of course combination therapies may also be employed within the discretion of the treating physician. Indeed, the above-described preventative therapies may include combinations of the local agent with systemic treatments and/or surgical interventions, including conventional radiation, prophylactic partial mastectomies (with local treatment of remaining ducts), herbal treatments, nutritional supplements, meditation, yoga, and/or dietary changes. The prophylactic methods may also be repeated periodically to continue to suppress progression of a disease state.

In another embodiment of the prophylactic method, the ductal epithelium of an exocrine gland, such as a mammary gland, is treated prophylactically for cancer so as to inhibit the formation of cancer of ductal epithelial origin. The method comprises contacting, preferably by ductal cannulation, the ductal epithelium of the exocrine gland with an epithelium-destroying agent to destroy less than all of the ductal epithelium so as to inhibit the formation of cancer of ductal epithelial origin. Preferably, up to about 70%, 80%, 85%, 90%, 95% or 100% of the ductal epithelium is destroyed. In some embodiments, cells underlying the ductal epithelium may also be targeted, based on the selective permeability of the epithelial lining to different agents.

In one embodiment, the epithelium-destroying agent is preferably a vector comprising a thymidine kinase gene, such as that from Herpes simplex, and ganciclovir, which can be brought into contact with the ductal epithelium by any suitable means, preferably by ductal cannulation or by systemic administration, a vector comprising a HPRT gene and HAT nucleotide, which can be brought into contact with the ductal epithelium by any suitable means, preferably by ductal cannulation or by systemic administration, a vector comprising a gene, which upon transformation of a cell of the ductal epithelium and expression therein, induces apoptosis or death of the transformed cell, such as bclxs, ethanol, or a cytolytic virus, such as Vaccinia virus.

The above method can additionally comprise the administration of a cytokine or hematopoietic growth factor, such as GM-CSF. The GM-CSF can be brought into contact with the ductal epithelium of the mammary gland by any suitable means, such as by ductal cannulation of GM-CSF or a vector comprising a gene encoding GM-CSF, in which case the vector can be the same vector as the one encoding the thymidine kinase, HPRT or apoptosis inducing gene, or it can be systemically administered.

This embodiment of the prophylactic method can be used to treat any exocrine gland. However, it is particularly useful in the treatment of the mammary gland.

The above-described prophylactic method of treating a mammary gland is particularly useful in treating a mammary gland in a mammal at risk for developing breast cancer. In some embodiments, the mammary gland can be characterized as one that has never had a tumor, one that had a tumor previously but the tumor is no longer detectable due to other prior therapeutic treatment, or one that has an incipient or occult tumor, preneoplasia or ductal hyperplasia. Normally, hyperplasias and incipient and occult tumors are not detectable by means of physical examination or radiography. Accordingly, the prophylactic method will find use in cases where there is reason to take some prophylactic measures, such as when there are known inherited factors predisposing to cancers, where there are suspicious lesions present in a breast with the potential for developing into a malignancy, where there has been exposure to carcinogenic agents in the environment, where age predisposes to a cancer, where cancer of another gland, e.g., the mammary gland of the contralateral breast, suggests a propensity for developing cancer, or where there is a fear or suspicion of metastasis.

Therapeutic Method—

The therapeutic method is a method of treating the ductal epithelium of an exocrine gland therapeutically for a disease that affects the ductal epithelium of the exocrine gland. The method comprises locally contacting, preferably by ductal cannulation, the ductal epithelium of the exocrine gland with an agent that destroys at least a portion of the epithelial cells of the ductal epithelium affected by the disease.

In one embodiment of the therapeutic method, the ductal epithelium of a mammary gland is locally treated so as to destroy cancerous and noncancerous cells of the ductal epithelium thereby having a therapeutic effect that may include killing hyperproliferative, transformed, and/or neoplastic epithelial cells, shrinking tumors, reducing the number of tumors (or tumor burden) and inhibiting the spread of cancer. The method comprises locally contacting, preferably by ductal cannulation, the ductal epithelium of the mammary gland with an agent adapted to destroy epithelial cells, which treatment need not, and preferably does not, specifically target cancerous cells. The agent preferably is a conventional chemotherapeutic agent, e.g., the cytotoxins selected from the group consisting of genistein, okadaic acid, 1-β-D-arabinofuranosyl-cytosine, arabinofuranosyl-5-aza-cytosine, cisplatin, carboplatin, actinomycin D, asparaginase, bis-chloro-ethyl-nitroso-urea, bleomycin, chlorambucil, cyclohexyl-chloro-ethyl-nitroso-urea, cytosine arabinoside, daunomycin, etoposide, hydroxyurea, melphalan, mercaptopurine, mitomycin C, nitrogen mustard, procarbazine, teniposide, thioguanine, thiotepa, vincristine, 5-fluorouracil, 5-fluorocytosine, adriamycin, cyclophosphamide, methotrexate, vinblastine, doxorubicin, leucovorin, taxol, anti-estrogen agents such as tamoxifen, intracellular antibodies against oncogenes, the flavonol quercetin, Guan-mu-tong extract, retinoids, nontoxic retinoid analogs, and monoterpenes. In preferred embodiments, the cytotoxic agent comprises: carboplatin or pegylated lipososmal doxorubicin. In alternative embodiments, the agent may comprise a vector comprising a thymidine kinase gene, such as a Herpes simplex thymidine kinase gene, and ganciclovir, a vector comprising a HPRT gene and HAT nucleotide, a cytolytic virus, such as a Vaccinia virus, or ethanol. The method can additionally comprise contacting the ductal epithelium with a cytokine or hematopoietic growth factor, such as GM-CSF.

In the therapeutic method, the epithelial-destroying agent should destroy all of the diseased or malignant epithelium. In addition, the ductal epithelium immediately surrounding the diseased/malignant epithelium also preferably should be destroyed. Non-epithelial tissues may also be targeted.

Combined Therapeutic/Prophylactic Method—

A method is also provided for treating the ductal epithelium of a mammary gland both therapeutically and prophylactically for cancer. The method comprises administering to the mammary gland any conventional therapeutic regimen known and used in the art. Examples of such methods include surgical removal of the cancerous tissue, radiation therapy and systemic chemotherapy. The combination method further comprises locally contacting, either before, concomitantly with or subsequent to the conventional therapeutic treatment, the ductal epithelium of the mammary gland, e.g., by ductal cannulation, with an agent that destroys or prevents from growing at least some epithelial cells along the ductal epithelial lining, which preferably does not specifically target cancerous cells, so as to render dead or harmless any remaining cancerous cells and noncancerous cells, thereby treating, reducing and/or preventing the cancer and/or inhibiting the spread of cancer. The agent may be a cytotoxin (e.g., conventional chemotherapy agent) or a vector comprising a thymidine kinase gene, such as a Herpes simplex thymidine kinase gene, combined with ganciclovir, a vector comprising a HPRT gene combined with HAT nucleotide, a cytolytic virus, such as a Vaccinia virus, or ethanol. The method can additionally comprise contacting the ductal epithelium with a cytokine or hematopoietic growth factor, such as GM-CSF.

Alternative Prophylactic & Therapeutic Methods—

Although some preferred embodiments have been described above, the methods can be used to treat the ductal epithelium of any exocrine gland, as well as non-epithelial cells of any exocrine glands. Examples of exocrine glands, other than the mammary gland, which can be treated with the present inventive methods include, among others, the prostate, liver, gall bladder, pancreas, kidneys, sweat glands, and salivary glands. The methods are especially useful in the prophylactic and therapeutic treatment of mammary glands.

Similarly, the methods can be used to treat an exocrine gland for any disease that affects the exocrine ductal epithelium. The methods are particularly useful in the treatment of cancer, including the stages of hyperplasia, adenoma, carcinoma in situ, and carcinoma, of ductal epithelial origin.

Any method can be used to destroy the ductal epithelium and/or non-epithelial cells/tissues. It is preferred, however, in some embodiments, that the destruction is limited to the ductal epithelium or a part thereof.

Any method of contacting the ductal epithelium can be used to effect local treatment. Preferably, ductal cannulation or catheterization is used, as described above. Although any duct or lobule can be cannulated, it is preferred that the central canal or duct be cannulated. Ductal cannulation also enables direct injection of a tumor mass, if desired.

Any epithelium-destroying agent can be used to destroy the ductal epithelium. In some embodiments, it is preferred that the agent should not destroy cells other than cells of the ductal epithelium and should not result in side effects, the adversity of which may outweigh the benefits of destruction of the ductal epithelium.

Examples of agents that can be used in the context of the prophylactic and therapeutic methods of the invention include cytotoxic agents. Any cytotoxic agent known in the art and suitable for contacting the ductal epithelium of an exocrine gland of a mammal can be used. In addition to ethanol and GCV described above, other examples of cytotoxic agents and their prodrugs include genistein, okadaic acid, 1-β-D-arabinofuranosyl-cytosine, arabinofuranosyl-5-aza-cytosine, cisplatin, carboplatin, actinomycin D, asparaginase, bis-chloro-ethyl-nitroso-urea, bleomycin, chlorambucil, cyclohexyl-chloro-ethyl-nitroso-urea, cytosine arabinoside, daunomycin, etoposide, hydroxyurea, melphalan, mercaptopurine, mitomycin C, nitrogen mustard, procarbazine, teniposide, thioguanine, thiotepa, vincristine, 5-fluorouracil, 5-fluorocytosine, adriamycin, cyclophosphamide, methotrexate, vinblastine, doxorubicin, leucovorin, taxol, anti-estrogen agents such as tamoxifen, intracellular antibodies against oncogenes, the flavonol quercetin, Guan-mu-tong extract, retinoids such as fenretinide, nontoxic retinoid analogues such as N-(4-hydroxyphenyl)-retinamide (HPR), and monoterpenes such as limonene, perillyl alcohol and sobrerol. Preferably, the agent is locally administered, especially if administration of the agent is accompanied by toxic side effects. Otherwise, the agent can be administered by any suitable route, such as systemic administration. In some embodiments, the same cytotoxic agent may be administered locally via ductal cannulation and systemically, wherein the two routes of administration are provided in any order or concomitantly.

A cytolytic virus also can be used as an agent. Any cytolytic virus can be used as long as the organism mounts a rapid immunological response to it such that the virus cannot cause disease if it escapes the ductal epithelium. Examples of cytolytic viruses include Vaccinia viruses and Sindbis viruses, which can also be used as vectors. Preferably, a Vaccinia virus is used. Due to lack of mucosal immunity, Vaccinia infectious particles enter and lyse the breast epithelial cells, yet stromal immunity destroys the particles as soon as they leave the ductile tree of the exocrine gland, thereby preventing cytolysis beyond the ductal epithelium. The advantages of Vaccinia administration are that it eliminates the need for high titer virus, the need to induce cell division in the breast, and the need to administer a drug to effect cell death.

A vector comprising a suicide gene also can be used as an agent, in conjunction with an agent that destroys the ductal epithelium of an exocrine gland. The vector comprising a suicide gene, upon transformation of a cell of the ductal epithelium and expression therein, renders the transformed cell sensitive to the epithelium-destroying agent, increases the sensitivity of the transformed cell to the agent, converts the agent from a prodrug to an active drug, activates the conversion of the agent from a prodrug to an active drug, enhances the effect of the agent or, itself, produces a protein that is cytotoxic. A preferred suicide gene for use in the present inventive methods is the one described above, i.e., a thymidine kinase, such as the one from Herpes simplex, which phosphorylates GCV, which, in turn, inhibits DNA replication. Another example of a suicide gene is cytosine deaminase, which is used in conjunction with 5-fluorocytosine. If the vector comprising the suicide gene is administered locally to the ducts, the cytotoxic agent or precursor can be administered systemically, since only transfected cells will be affected. In this regard, the bystander effect, i.e., the death of neighboring uninfected cells, presumably due to transfer of toxic byproducts through gap junctions between cells in the same compartment, obviates the need for every cell in the ductal epithelium, which is to be destroyed, to be infected. However, sufficient time must be allowed between contacting the ductal epithelium with the suicide gene and the prodrug, for example, to achieve efficient killing of the breast epithelial cells.

A vector comprising an apoptosis-inducing gene also can be used as an agent that destroys the ductal epithelium of an exocrine gland (Vaux, Cell 76:777-779 (1994)). Examples of apoptosis-inducing genes include ced genes, myc genes (overexpressed), the bclxs gene, the bax gene, and the bak gene. The apoptosis-inducing gene causes death of transfected cells, i.e., by inducing programmed cell death. For example, the bclxs gene, bax gene, or bak gene can be used to inhibit bcl-2 or bcl-X_{L}, leading to apoptosis. Where necessary, a vector comprising an apoptosis-inducing gene can be used in combination with an agent that inactivates apoptosis inhibitors such as bcl-z, p35, IAP, NAIP, DAD1 and A20 proteins.

Suicide and apoptosis genes can be administered by way of a viral vector, such as an adenoviral or retroviral vector. Adenoviral vectors enable the generation of high titer recombinant viruses (10¹¹/ml) and the efficient transduction of postmitotic cells because adenoviral DNA exists as an episome in the nucleus (Verma, Molecular Medicine 1:2-3 (1994)).

According to one preferred embodiment, the gene can be under the transcriptional regulation of a Rous sarcoma viral promoter. Alternatively, it can be under the control of an epithelial tissue-specific or cell-specific promoter.

Uptake of recombinant virus can be facilitated by pretreatment or simultaneous treatment with polybrene or, for example, in the case of a retrovirus, attachment of the functional fragment of an antibody to the viral particle.

In another embodiment, the apoptosis gene or suicide gene can be present in a recombinant microorganism, which will express the gene. One particularly preferred microorganism for this purpose is the bacterium Listeria monocytogenes.

Other methods known in the art for introduction of raw DNA into cells can be used in the methods of the invention. Alternatively, liposomes, complexes between polypeptide ligands for receptors on mammary ductal epithelial cells, including complexes of antibodies and functional fragments thereof, and plasmids can be used (Mulligan, Science 260:926-931 (1993)). Epithelial cell-specific promoters, such as whey acidic protein (wap), can be used to target expression of a given gene, e.g., a suicide gene, in ductal epithelial cells. Use can also be made of wild-type tumor suppressor genes, such as p53 or Mcs-1 (rat), homeobox genes expressed in normal cells but not in cancerous cells, and the maspin gene.

Additionally, the ductal epithelium can be contacted with an agent to effect the scavenging of epithelial cells destroyed in accordance with the invention, e.g., a cytokine/growth factor. Suitable cytokines/growth factors include GM-CSF, G-CSF, IL-2, IL-4, IL-6, IL-7, hCG, TNF-α, INF-α and INF-γ. Such factors can be contacted with the ductal epithelium directly or by expression of a vector comprising a gene encoding the factor, in which case the vector can be the same one that comprises a suicide gene, for example. The factors stimulate a potent, long-lasting, and specific cell immunity, requiring both CD4 and CD8 cells. The immune response is designed to scavenge destroyed ductal epithelial cells by generating autoimmunity towards epithelial cell antigens.

The ductal epithelium is preferably contacted with the agent by introduction of the agent through the central canal or duct of the exocrine ductal epithelium, such as by ductal cannulation. However, in the case of the mammary gland, for example, there are 6-9 major ducts that emanate from the nipple and serially branch into other ducts, terminating in lobulo-alveolar structures (Russo et al. (1990), supra). Accordingly, in some circumstances, such as those in which even more localized treatment is necessary or desired, for example, by the choice of anti-cancer agent, it may be preferable to contact the ductal epithelium of the exocrine gland through one of the other ducts or through a lobulo-alveolar structure as opposed to the central canal or duct. In this regard, ductal cannulation enables intratumoral injection.

The methods of the invention can be combined with other methods of prophylactic and therapeutic treatment in addition to those cited above, such as methods that target destruction of cancer cells, e.g., by targeting of cell-surface markers, receptor ligands, e.g., ligands to gastrin-releasing peptide-like receptors, tumor-associated antigens, e.g., the 57 kD cytokeratin or the antigen recognized by the monoclonal antibody GB24, the extracellular matrix glycoprotein tamascin, antisense oncogenes such as c-fos, homeobox genes that are expressed in cancer cells but not normal cells, tumor-infiltrating lymphocytes that express cytokines, RGD-containing peptides and proteins, which are administered following surgery, lipophilic drug-containing liposomes to which are covalently conjugated monoclonal antibodies for targeting to cancer cells, low fat diet, moderate physical exercise and hormonal modulation. For prostate cancer, anti-testosterone agents can be used as well as an inhibitor of cell proliferation produced by prostatic stromal cells and C-CAM, an epithelial cell adhesion molecule.

In some embodiments, immunotherapeutic agents may by used, such as HERCEPTIN™.

In some embodiments, agents can include aptamers, antibodies (including single chain antibodies), or bispecific T-cell engagers. These agents can be used alone or in combination with other desired agents, with appropriate carriers.

Aptamers include nucleic acid molecules that specifically bind to proteins and other biomolecules due to their defined 3d structure. In some embodiments, an aptamer can be delivered to a breast duct that is capable of binding to a surface antigen of a cancerous or precancerous cell in the breast duct, such as a cancerous or precancerous epithelial cell. Suitable aptamer can be obtained, for example with the SELEX process (see, e.g. “Smart Designed Aptamers Applications & Effective Design Options,” published by Gene Link at www.genelink.com/Literature/ps/Aptamer_PG_Ver3.1.pdf, last viewed Apr. 14, 2009, incorporated by reference herein in its entirety), or by other suitable techniques including those described in U.S. Pat. Nos. 5,756,291 (Griffin, et al.) and 7,329,742 (Doyle, et al), incorporated by reference herein in their entireties. Suitable aptamers can have therapeutic or prophylactic functionality and/or can be associated with agents, such as prophylactic or therapeutic agents or imaging agents. In some embodiments, suitable aptamers can be associated with a carrier to modify the aptamer's persistence in the breast duct, whether or not it is associated with other agents.

Antibodies, including those which bind to breast cancer and precancer cells can be delivered to a breast duct as described herein. In some embodiments, an antibody of the IgG or IgM class and binds to breast cancer or precancerous cells.

In some embodiments, an antibody can be a single chain antibody which is a single peptides that exhibit antibody-like binding characteristics, but without the Fab-Fc region with dual heavy chain-light chain structure. In some embodiments, single chain antibodies can include two antibody heavy chain segments incorporated into the structure of a single peptide, as described in U.S. Pat. No. 4,704,692 (Ladner), incorporated herein by reference in its entirety. In some embodiments, single chain antibodies can include two or more antigen binding sites within a single peptide sequence, to provide specific binding to desired antigens on particular cells. In some embodiments, a single chain antibody contains antigen binding sites to bind to breast cancer cells or precancerous cells.

In some embodiments, an antibody or single chain antibody is associated with a cytotoxic agent, as described herein, or an imaging agent, as described herein, and delivered to a breast duct for therapeutic, prophylactic, or imaging uses. In some embodiments, an antibody of single chain antibody can be associated with a carrier to provide sustained deliver or for persistence in the breast duct.

Bispecific (or bifunctional) T-cell engagers (BiTE) include compounds that serve to redirect T-cells for antigen-specific elimination of antigens. BiTE molecules have a first antigen binding domain that binds to a T-cell antigen, and a second antigen-binding domain that can bind to an antigen on the target cell. BiTE molecules as well as methods of using BiTE molecules are described in U.S. patent application Ser. No. 11/645,290 (Hammond, et al.), U.S. patent application Ser. No. 11/684,979 (Bruckheimer, et al.), U.S. patent application Ser. No. 10/863,729 (Kinch, et al.), U.S. patent application Ser. No. 10/918,685 (Wands, et al.), U.S. patent application Ser. No. 10/565,771 (Kinch), U.S. patent application Ser. No. 11/259,133 (Dall'Acqua, et al.), U.S. patent application Ser. No. 11/953,653 (Kinch), U.S. patent application Ser. No. 10/854,326 (Kiener, et al.), U.S. patent application Ser. No. 11/263,230 (Losonsky, et al.), U.S. patent application Ser. No. 11/259,266 (Kinch, et al.), U.S. patent application Ser. No. 10/628,088 (Young, et al.), U.S. patent application Ser. No. 11/004,794 (Kinch), U.S. patent application Ser. No. 11/063,574 (Mao, et al.), U.S. patent application Ser. No. 11/633,729 (Chang, et al.), and U.S. patent application Ser. No. 10/461,863 (Oliver, et al.), all incorporated by reference herein in their entireties.

In some embodiments, one or more BiTE molecules can be delivered to a breast duct with our without a carrier. In some embodiments, the BiTE molecule will have an antigen-binding domain that binds to an antigen on a cancerous or precancerous cell in the breast duct. In some embodiments, BiTE molecules are associated with a carrier to provide a sustained release of BiTE molecules over the course of one day, three days, one week, two weeks, four weeks, six weeks, eight weeks, or longer. In some embodiments, the BiTE is covalently linked to a carrier by a bond that is slowly hydrolyzed under conditions present in the breast duct.

Other agents, such as vitamins, hormones, nutritional supplements, herbal extracts, etc. may also be included in the gel composition, which may promote health of the treated breast.

### Hydrogels

In some embodiments, the delivery of the agent will be facilitated by the introduction of a hydrogel. The property of preferred gel compositions are that they are low viscosocity on infusion, but following a period of time, become more viscous, and then a solid material or a hydrogel. By using a biodegradable material, it may be demonstrated that the cycle of repeated infusion and degradation of the biomaterial within a duct will allow for a greater volume of material to be infused into the duct over time.

Hydrogels are three dimensional, hydrophilic, polymeric networks which swell in water without dissolving and retain large quantities of water. They typically comprise both natural polymers such as starches and cellulose derivatives and synthetic polymers and copolymers such as polyethylene glycol and poly(glutamic acid). The networks within the hydrogel can be crosslinked either chemically or physically by cohesive bonds such as ionic interaction, hydrogen bonding or hydrophobic interactions. Alginates and chitosans are examples of polymers that are cross-linked by ionic interactions. Hydrogels are suitable for numerous applications such as implants, contact lenses, membranes for isosensors and drug delivery devices.

Suitable hydrogels for use in accordance with aspects of the present invention can be formed from synthetic polymers such as polyethylene glycol, polyethylene oxide, polyvinyl alcohol, polyvinyl pyrrolidone, polyacrylates, poly(ethylene terephthalate), poly(vinyl acetate), and copolymers and blends thereof, as well as natural polymers such as cellulose and alginate.

Additional preferred materials include those having a polymeric component, including, but not limited to, polysaccharides, polypeptides, polyethers, and derivatives thereof. Frequently, the individual polymeric strands can interact with themselves and other strands through van der Waals, ionic, or covalent linkages, either directly or through additional chemical compounds. Preferred polysaccharides include alginates, gelan gum (deacylated, high acetyl, low acetyl, high glyceryl, low glyceryl, intermediate levels of acetyls and/or glyceryl, and mixtures thereof), cellulosics, pectins, cellulose (including microcrystalline cellulose, and bacterial cellulose), agar, fucoidan, chitan, chitosan, carageenans, hyaluronic acid, propylene glycol alginate: polysaccharides containing glucose, fructose, galactose, guluronate, mannuronate, 6-carbon sugars, acids of 6-carbon sugars, 5-carbon sugars, and acids of 5-carbon sugars; etc.

In some embodiments, a polymer produced from a poly(ethylene glycol) (PEG) derivative can be used, for example, a,co-dihydroxy-PEG or a,w-diamino-PEG, but other derivatives are embraced herein. Examples of such polymers with particular molecular weights include a co-dihydroxy-PEG3,400; a,w-dihydroxy-PEG1,000; a, co-diamino-PEG.-3,400; and a,a)-diamino-PEG1,000. PEG is known to be a particularly nontoxic polymer. These derivatized PEG subunit polymers may be used as amino- and hydroxy-containing polymers for cross-linking, or may be further derivatized, for example, to prepare the polymer on which at least two thiol groups are present by derivatization with thiomalic acid. Thiomalic acid (also known as mercaptosuccinic acid) may be replaced by dimercaptosuccinic acid, thereby doubling the number of sites available for cross-linking. Increasing the extent of cross-linking the matrix results in a gel with smaller pores.

In some embodiments, a PEG hydrogel can be utilized, such as one that can be formed by crosslinking PEG scaffolds directly or through a crosslinking compound, resulting in thioether or disulfide linkages between scaffold molecules or between scaffold and crosslinking molecules. Suitable crosslinking compounds, when used, include compounds having two or more functional groups, such as thiol groups or thiol-reacting groups, or a combination of thiol and thiol reactive groups. In some embodiments, at least one of these groups can be blocked to allow the other(s) to react, and then unblocked to allow the previously blocked groups to react.

In some embodiments, a nanocarrier can be incorporated into the hydrogel by incorporating previously produced nanocarriers into the hydrogel reaction system at an appropriate point, such as to PEG scaffold solution or crosslinker solution, prior to mixing of the PEG scaffold with crosslinker, or after mixing of the PEG scaffold with crosslinker, but before an undue amount of crosslinking has occurred.

The hydrogel composition, such as PEG-based composition, can provide a platform technology suitable for use with various types of drugs to be delivered. The drugs can be either physically entrapped or modified drugs with cleavable bonds can be physically incorporated or covalently linked into the hydrogel to provide controlled release, which is otherwise not possible for highly hydrophilic drugs which traverse easily through the gel.

### Electrical Characteristic Analyzer

FIG. 9 illustrates an equivalent circuit for tissue, such as tissue including a milk duct. An analyzer 140 having electrical contact with the tissue can distinguish different types of tissue via electrical characteristics. For example, electrical characteristics can be used to distinguish between a milk duct and other tissue. This can be used as a guide for the delivery of a therapeutic agent.

In the illustrated example of FIG. 9, the biological tissue is electrically modeled as a parallel combination of a resistance R and a capacitance C (parallel RC circuit). Typically for biological tissue, the electrical values are relatively high for resistance R, and are relatively low for the capacitance C. For example, when probing external to a body in a non-invasive manner, the resistance R can be the resistance associated with the skin of the body.

Identification of biological tissue using electrical characteristics can be a relatively difficult task. One difficulty encountered is that electrical characteristics of biological tissue can vary in a very broad range. It can be relatively difficult to accommodate a very wide range of electrical characteristics for a practical analyzer. Moreover, when the biological tissue is the living tissue of a patient, electrical tests for characterization should not harm the patient.

One embodiment of the invention uses relative electrical characteristics to characterize tissue. The use of relative electrical characteristics overcomes the difficulties associated with making absolute measurements of electrical characteristics having a very broad range. In the illustrated embodiment, the electrical characteristics are determined using two different frequencies for a test signal. For example, a first test signal with a relatively pure frequency of 25 kHz is used, and then a second test signal with a relatively pure frequency of 100 kHz is used for a frequency ratio of 4:1. While illustrated in the context of 2 separate signals with a frequency ratio of 4:1, the skilled artisan will appreciate that the principles and advantages described herein are applicable to other frequency ratios, e.g., a frequency ratio of n, more than 2 signals and/or frequencies, and the like. Other and/or additional frequencies and frequency ratios will be applicable. In addition, a test signal carrying two or more frequencies can also be evaluated to yield the relative measurements.

### Magnitude Ratio |Y|

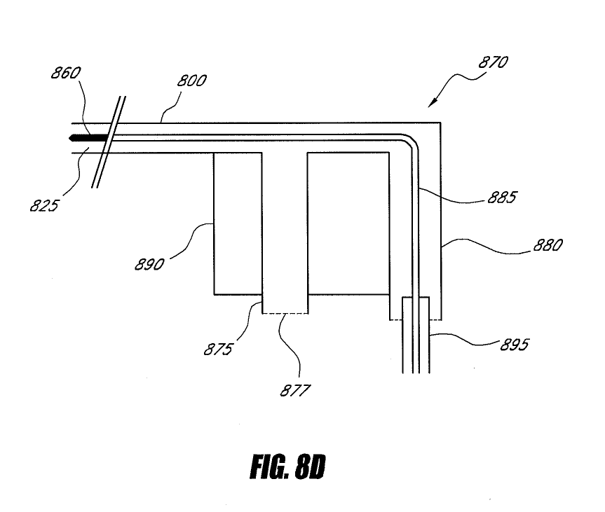

FIG. 10 is a chart illustrating magnitude ratios of admittance for a variety of fixed conductances, a 4:1 frequency ratio, and a range of capacitance values. The concept of admittance Y, which is the inverse of impedance Z, will be used in the following analysis. Admittance Y and impedance Z can be readily converted. With reference to FIG. 9, the admittance Y of the model for the biological tissue is expressed in Equation 1.

Y=G+jB (Eq. 1)

As expressed in Equation 1, the admittance is the sum of a real component (conductance G) and an imaginary component (susceptance B). For the model of the body tissue, the conductance G is the inverse of the resistance R and the susceptance is ωC, where radian frequency ω is 2πf, where f is frequency. The susceptance is frequency dependent. A magnitude of the ratio of admittance (“magnitude ratio”) |Y| is expressed in Equation 4. The admittance of the lower frequency, e.g., 25 kHz, is represented by Y₁ (Equation 2) and the admittance of the higher frequency, e.g., 100 kHz, is represented by Y₂. In the following equations, the lower frequency is f or ω, and the higher frequency is 4f or 4ω.

$$\begin{matrix}{Y}_{1} = G + jω C & ( Eq . 2 ) \\ {Y}_{4} = G + j4ω C & ( Eq . 3 ) \\ \vert Y \vert = \vert \frac{{Y}_{4}}{{Y}_{1}} \vert = \frac{\sqrt{{G}^{2} + 16 {ω}^{2} {C}^{2}}}{\sqrt{{G}^{2} + {ω}^{2} {C}^{2}}} & ( Eq . 4 )\end{matrix}$$

FIG. 10 illustrates magnitude ratios for a variety of conductances G. A range of capacitance C from 1 picofarad (pF) to 1000 pF is shown along the x-axis. The magnitude ratio |Y| is shown along the y-axis with a scale ranging from 1 to 4. From top to bottom, four example curves are shown with conductance G equal to 5 microsiemens (μS), 50 μS, 500 μS, and 5 millisiemens (mS). For all curves, a lower frequency of 25 kHz and a higher frequency of 100 kHz are used. The curves are plotted as the value for capacitance C is varied.

As illustrated by the curves, the magnitude ratio |Y| remains in a relatively narrow 4:1 range even though, in this example, the capacitance C varies by a factor of 1000 and the range of values shown for conductance also ranges by a factor of 1000. As illustrated in the chart of FIG. 10 and by inspection of Equation 4, as the capacitance C goes to zero, the magnitude ratio |Y| goes to 1. As the capacitance C goes to infinity, the magnitude ratio |Y| goes to 4. As the magnitude ratio |Y| is a monotonic function, the range of values for the magnitude ratio |Y| is 4:1, for the case with a frequency ratio of 4.

The magnitude ratio |Y| offers relatively good sensitivity to a wide range of conductance G and capacitance C. This sensitivity, represented graphically by sloped regions of the curves, can be useful to distinguish between different types of tissue. However, as illustrated by the curve representing a conductance G of 5 mS (FIG. 2), for relatively high values of conductance G (less resistance), the magnitude ratio |Y| exhibits relatively little sensitivity to capacitance values. Accordingly, the magnitude ratio |Y| can be supplemented by other techniques.

### Phase Ratio θᵣ

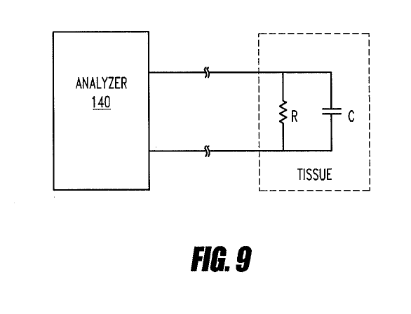

FIG. 11 is a chart illustrating phase ratios θᵣ for admittance Y for fixed conductances G, a 4:1 frequency ratio, and a range of capacitance values. The phase ratio θᵣ represents the ratio between the phase relationship at the higher frequency θ_{4f} and the phase relationship at the lower frequency θ_{f}. In one embodiment, with an AC voltage source provided as an output of the analyzer 140, the phase relationship corresponds to the phase of the current relative to the phase of the voltage.

A range of capacitance C from 1 pF to 1000 pF is shown along the x-axis. A phase ratio θᵣ is shown along the y-axis ranging in scale from 1 to 4. From top to bottom, four example curves are shown with conductance G equal to 5 μS, 50 μS, 500 μS, and 5 mS. For all curves, a lower frequency of 25 kHz and a higher frequency of 100 kHz are used. The curves are plotted as the value for capacitance C is varied.

The curves represent the plotting of the phase ratio θᵣ as expressed in Equation 5. In Equation 5, the lower frequency is f or ω, and the higher frequency is 4f or 4ω.

$${θ}_{r} = \frac{{θ}_{4 f}}{{θ}_{f}} = \frac{arctan ( \frac{4 ω C}{G} )}{arctan ( \frac{ω C}{G} )} \qquad ( Eq . 5 )$$

As illustrated in FIG. 11, even for a relatively wide 1000:1 variation in capacitance C and/or conductance G, the phase ratio θᵣ is maintained to a relatively narrow 4:1 ratio. As illustrated in FIG. 11, the phase ratio θᵣ offers relatively good sensitivity for a relatively high conductance G, such as 5 mS. With reference to earlier FIG. 10, the sensitivity of the magnitude ratio |Y| for the conductance G value of 5 mS is not particularly good, and thus the example illustrates that the magnitude ratio |Y| and the phase ratio θᵣ can supplement each other. In another example, the magnitude ratio |Y| provides relatively good sensitivity for a relatively low conductance G, such as 5 μS, in contrast to the relatively poor sensitivity for the phase ratio θᵣ for 5 μS of admittance (FIG. 11).

### Phase Difference θ_{d}

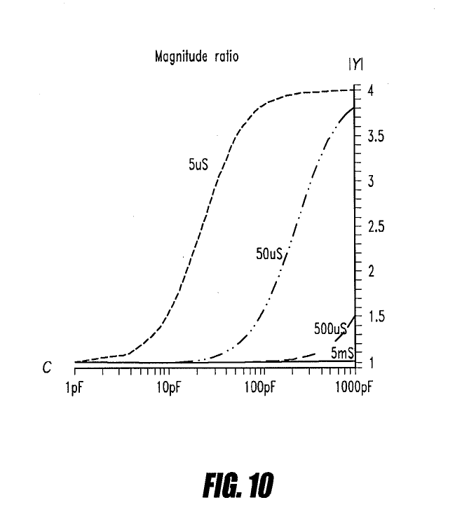

FIG. 12 is a chart illustrating phase differences θ_{d} for admittance for fixed conductances, a 4:1 frequency ratio, and a range of capacitance values. The phase ratio θ_{d} represents the difference between the phase relationship at the higher frequency θ_{4f} and the phase relationship at the lower frequency θ_{f}. In the illustrated embodiment, with an AC voltage source provided as an output of the analyzer 140, the phase relationship corresponds to the phase of the current relative to the phase of the voltage.

A range of capacitance C from 1 pF to 1000 pF is shown along the x-axis. A phase difference θ_{d} is shown along the y-axis ranging in scale from 0 to about 0.65 radians. The scale for the phase difference θ_{d} can vary depending on the values of the phase relationships θ_{4f} and θ_{f}. The phase difference θ_{d} is expressed in Equation 6 and is plotted in FIG. 12.

$${θ}_{d} = arctan ( \frac{4 ω C}{G} ) - arctan ( \frac{ω C}{G} ) \qquad ( Eq . 6 )$$

For all curves, a lower frequency of 25 kHz and a higher frequency of 100 kHz are used. The curves are plotted as the value for capacitance C is varied. In Equation 6, the lower frequency is f or ω, and the higher frequency is 4f or 4ω. As illustrated by FIG. 12, the phase difference θ_{d} is not necessarily monotonic. The first peak from the left corresponds to a conductance G of 5 μS. The second peak from the left corresponds to a conductance of 50 μS. The other two curves that are generally rising from left to right for the selected range correspond to conductances of 500 μS and to 5 mS, with the 500 μS curve higher than the 5 mS curve.

While the phase difference θ_{d} can be non-monotonic, the phase difference θ_{d} can still be useful to distinguish between different types of biological tissue in combination with the magnitude ratio |Y| (FIG. 10), the phase ratio θᵣ (FIG. 11), or combinations thereof.

As will be illustrated in the test data below, the various tests complement each other relatively well. The magnitude ratio |Y| (FIG. 10) and the phase difference θ_{d} (FIG. 12) can distinguish wide ranges of capacitance in tissues when the resistance R, such as resistance of the skin, is relatively high, such as over 20 kohms. The phase ratio θᵣ (FIG. 11) can distinguish wide ranges of capacitance in tissues when the resistance R is relatively low, such as less than 20 kohms.

### Chart of Measured Data

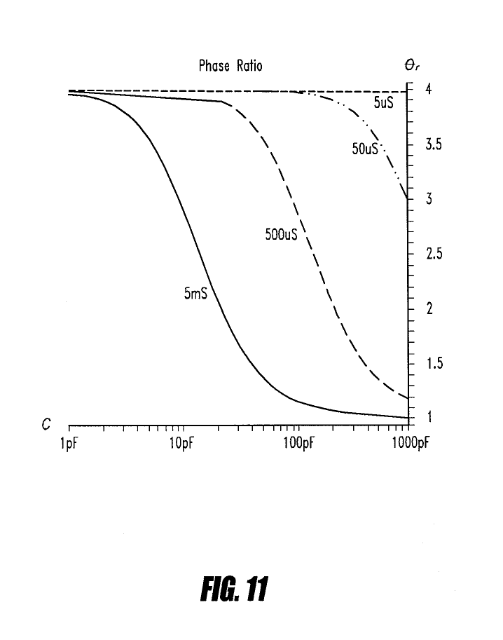

FIG. 13 is a 3-dimensional chart (x, y, z) of actual test data taken from human breast tissue removed via mastectomy. A magnitude ratio |Y| is shown along the x-axis, and the scale ranges 0.5 to 4.5. A phase ratio θᵣ is shown along the y-axis, and the scale ranges from 1 to 3.5. A phase difference θ_{d} is shown along the z-axis, and the scale ranges from 30 to 90 degrees. While illustrated in the context of distinguishing ductal orifices such as milk ducts of human breasts, the principles and advantages are applicable to distinguishing other types of tissue.

The data shown in FIG. 13 was taken by probing on the surface of human breast tissue (see FIG. 1). Data enclosed by ellipse 1302 corresponds to the electrical characteristics with the probe 120 (FIG. 1) contacting a ductal orifice O (FIG. 1). Other data shown in FIG. 13 corresponds to probe contact with other portions of the nipple N. As illustrated by the data, human tissue can vary dramatically in electrical characteristic.

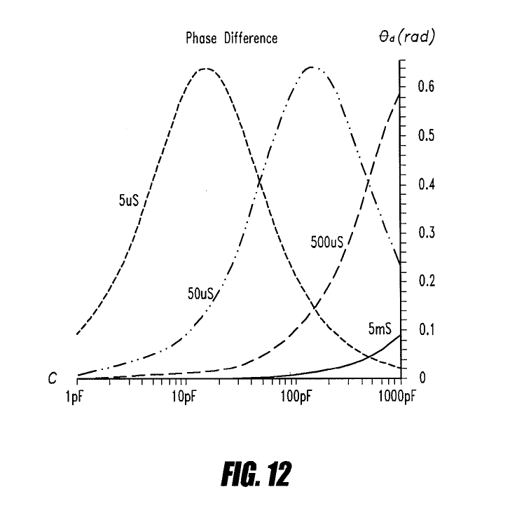

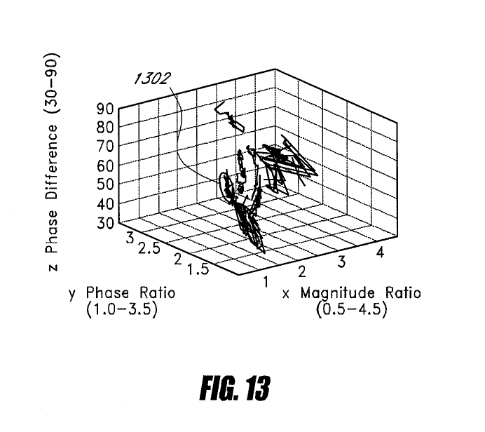

FIGS. 14 and 15 illustrate two views of the same example of using a multi-dimensional threshold to distinguish different types of tissue. The same data from FIG. 13 is shown in FIGS. 14 and 15, but from different perspective views. In the illustrated example, two criteria are used to identify tissue. In the illustrated embodiment, a first criterion is that the observed phase ratio θᵣ (y-axis) be greater than 1.585. The first criterion is not shown in FIGS. 14 and 15. It will be understood that the criterion used can vary with the frequencies utilized, the frequency ratio, and the like. In addition, the first criterion can be a pre-determined constant as shown in the illustrated example, or can be dependent on one or more other variables as shown with a second criterion.

The second criterion, based on a relationship between the magnitude ratio |Y| (x-axis) and the phase difference θ_{d} (z-axis) is represented graphically in FIGS. 14 and 15 as a sloped two-dimensional plane. Both FIGS. 14 and 15 represent the same plane. The illustrated plane is described by Equation 7.

z=74.6156x−15.7 (Eq. 7)

In Equation 7, z represents the phase difference θ_{d}, and x represents the magnitude ratio |Y|. The combination of the two criteria efficiently identifies which probed electrical characteristics correspond to ductal orifices O (FIG. 1) and which correspond to other tissue. In the illustrated embodiment, the measurements having a phase ratio θᵣ greater than 1.585 (first criterion) and having a phase difference θ_{d} (z) greater than, that is above in FIGS. 14 and 15, the equality expressed in Equation 7 are determined to be ductal orifices O (FIG. 1) and those outside of the two criteria are determined to be other tissue, such as skin surfaces, other glands, folds, and the like. These criteria combine to form a 3-dimensional threshold. While the particular second criterion expressed in Equation 7 is linear, non-linear combinations are also applicable and will be readily determined by one of ordinary skill in the art. Such non-linear combinations can be represented as curved surfaces, spheres, cylinders, cones, planes with 3 components, etc.

One advantage of the illustrated technique is that the relative formulations narrow the criteria range(s) and simplify the determination of tissue. Thus, for example, ductal orifices O (FIG. 1) can be identified with relatively little to no special calibration procedures. The technique is relatively immune to variability among breast tissue. In one embodiment, identification that a ductal orifice (O) (FIG. 1) has been located is indicated to the operator of the analyzer 140 by, for example, an audio indication, a visual indication, a vibrating indication, or a combination of the same.

### Detailed Embodiment

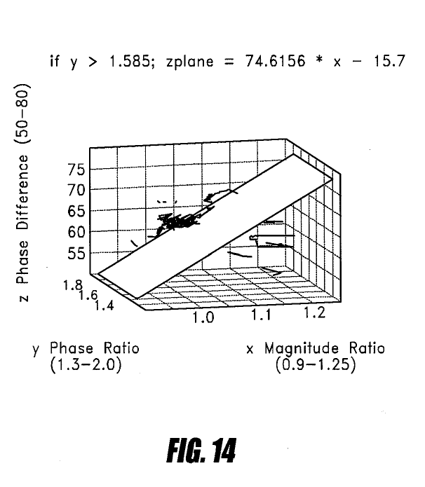

FIG. 16 is a block diagram of the analyzer 140 according to an embodiment of the invention. It will be appreciated by the skilled practitioner that the analyzer 140 can be modified in a variety of ways. For example, in another embodiment, various functional blocks can be combined, can be separated, and the like.

The analyzer 140 can include a circuit analyzer 1600, a controller 1670, a signaling audio transducer 1680, and a lookup table 1690. The circuit analyzer 1600 can include a programmable direct digital synthesizer (DDS) and digital-to-analog converter (DAC) 1610, a relatively low-output impedance amplifier 1620, a current sense amplifier 1630, an analog-to-digital converter (ADC) 1640, and a Fourier Transform processor 1650, e.g., discrete Fourier Transform processor. A coherent sampling timing signal 1660 can be provided by a clock or oscillator. In one embodiment, the circuit analyzer 1600 is embodied in a single network analyzer integrated circuit, such as an AD5933 available from Analog Devices Inc.

In the illustrated embodiment, the circuit analyzer 1600 is coupled to the patient body via, for example, the reference electrode 130 and the test electrode probe 120. For example, the reference electrode 130 can be coupled to the skin between the breasts, and the test electrode probe 120 moved to different locations by the operator. The test electrode probe 120 can include a catheter to inject an agent into a duct. However, it will be appreciated that the analyzer 140 is also useful to distinguish tissue for identification and can be used without a catheter integrated with the test electrode probe 120.

For control, the circuit analyzer 1600 is coupled to the controller 1670. For example, the controller can be a microprocessor, microcontroller, or the like that executes software or firmware. The controller 1670 can alternatively be implemented by dedicated hardware logic, or by a combination of both hardware and software. In one embodiment, the controller 1670 is an ARM7 processor available from ARM Holdings and controls the circuit analyzer 1600 via firmware control. The firmware is typically stored in nonvolatile memory, such as Flash memory.

For example, the controller 1670 can configure the direct digital synthesizer of the DDS and DAC 1610 by loading information for the particular waveforms to be generated by the DDS. These can be, for example, 25 kHz and 100 kHz sine waves. It will be understood that other frequencies can be used, that the 25 kHz and 100 kHz signals can be combined into a single signal, and that the signal does not have to be a sine wave. For example, a square wave can be used. In one embodiment, the signal that is used is coherent such that phase information can be extracted relatively efficiently. In the illustrated embodiment, under the control of the controller 1670, the DDS alternates between the signals for 25 kHz and for 100 kHz relatively rapidly in comparison to the length of time that an operator would probe a particular point on the body such that both frequencies are tested for a probed point even though only one is expressed at a time. The signal from the DDS and DAC 1610 is buffered by the amplifier 1620, and then the signal is provided to the patient body via the reference electrode 130 or the test electrode probe 120. It will be understood that a current path for the signal is not formed for the signal until the operator both the reference electrode 130 and the test electrode probe 120 are contacting the body.

The operator moves the test electrode probe 120 around to pick up the electrical signal from the body, which is buffered by the current sense amplifier 1630. In the illustrated embodiment, the circuit analyzer 1600 generates a voltage source output, and the current passing through the body tissue is analyzed. In an alternative embodiment, the circuit analyzer 1600 can be configured to generate a current source output, and the voltage drop across the body tissue can be analyzed.

Returning now to the illustrated embodiment, the current passing through the body completes the series circuit between the amplifier 1620 and the current sense amplifier 1630. The current sense amplifier 1630 converts the current into a voltage output. An output of the current sense amplifier 1630 is provided to the ADC 1640, which provides a digital representation of the sensed current to the Fourier Transform processor 1650. In one embodiment, the Fourier Transform processor 1650 is a DFT processor as provided in an AD5933 chip. The Fourier Transform processor 1650 computes the magnitude and phase of the sensed current in relation to the voltage source. From this information, the admittance Y for the model described earlier in connection with Equation 1 can be calculated and both the real conductance component G, and the imaginary susceptance component B calculated for use in Equations 4, 5, and 6 (with B equal to ωC or to 4ωC as appropriate).

One advantage to using the Fourier Transform processor 1650 is that multiple frequencies, e.g., both f and 4f, can be analyzed at the same time. However, when single frequencies are used in an alternating fashion, simpler techniques can be used to determine magnitude and phase information. For example, peak detection or root-mean-square (RMS) computation techniques can be used to determine a magnitude of the sensed current. Offsets in time between zero-crossings or waveform peaks for the voltage output waveform and the sensed current waveform can be used to derive phase shifts between voltage and current. Moreover, it should be noted that phase and time offsets are related to each other based on frequency, and that either phase information or time offsets can be used for a given frequency. Equation 8 expresses the relationship between a phase θ and time offset t_{d} for a given frequency f.

θ=t_{d}f (Eq. 8)

The controller 1670 retrieves the magnitude and phase information from the Fourier Transform processor 1650 and computes the magnitude ratio |Y|, the phase ratio θᵣ, and the phase difference θ_{d}. The controller 1670 can compare these recovered parameters to the 3-dimensional criteria stored in the lookup table 1690.

A lookup table 1690 or 3-dimensional lookup space can store thresholds for applicable criteria, such as the 2 criteria described earlier in connection with FIGS. 14 and 15. The lookup table 1690 can also be stored in nonvolatile memory, such as Flash memory or a hard disk. Alternatively, the lookup table 1690 can be calculated upon initialization by the controller 1670 from stored parameters (such as from parameters for Equation 7) and stored in any type of memory, including volatile memory, such as DRAM or SRAM. The contents of the lookup table can vary depending on which type of tissue is to be distinguished. In one embodiment, a variety of lookup tables 1690 are stored in the analyzer 140, and the controller 1670 can select the appropriate lookup table 1690 to use based on the type of tissue to be distinguished by the operator. In an alternative embodiment, the lookup table 1690 is not used and rather, the controller 1670 evaluates thresholds such as thresholds from Equation 7 directly.

Upon discrimination of tissue type, the controller 1670 can be configured by the firmware to activate the signaling audio transducer 1680 to generate a sound such that an operator can easily determine when a ductal orifice O (FIG. 1) has been probed by the test electrode probe 120. The signaling audio transducer 1680 can be a device such as a piezo buzzer, speaker, or the like. Other forms of indication that can be used in addition to or in alternative to the signaling audio transducer 1680 include vibrators, visual indicators, such as a light, and the like.

### Frequency Ratio

While described above in connection with a frequency ratio of 4:1, other frequency ranges are applicable. To obtain relatively good resolution, a frequency ratio of at least about 4 is preferably utilized, but lower values such as 2 can be utilized. Higher frequency ranges than 4 can provide even better resolution. In one embodiment, the frequency ratio is between 4 and 10.

The frequencies can also vary widely. The upper limit of the higher frequency, e.g., 4ω or 4f, should be low enough such that the signal does not radiate. For example, one upper limit is 1 MHz. The lower limit of the lower frequency, e.g., ω or f, should be high enough for relatively good measurements of relatively small amounts of capacitance. In one embodiment, the lower limit of the lower frequency is above the audible hearing range for the comfort of the patient and/or operator. In addition, relatively low frequencies, such as below around 100 Hz, should not be used around the heart.

### Process

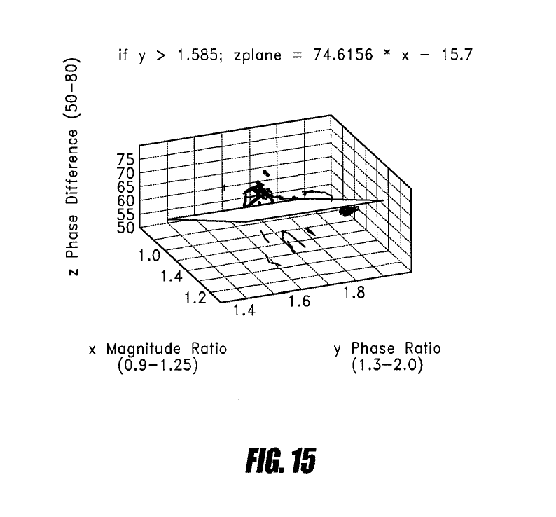

FIG. 17 illustrates a method for distinguish types of tissue based on electrical characteristics. The illustrated process can be modified in a variety of ways. For example, in another embodiment, various portions of the illustrated process can be combined, can be rearranged in an alternate sequence, can be removed, and the like. The illustrated process can be performed by the analyzer 140 (FIGS. 1 and 16). The probes 130, 120 (FIG. 1) for the analyzer 140 (FIGS. 1 and 16) can make contact with the patient's body independently of the illustrated process. In the illustrated example, the analyzer 140 (FIG. 16) has powered up, an appropriate lookup table 1690 (FIG. 16) has been generated or loaded, and the probe 130, 120 are in contact with the patient.

The process begins at a state 1710, wherein the electrical characteristics of the probed tissue are determined using at least two different frequencies. These frequencies can be provided sequentially (alternating when operating in a loop within the state 1710), or can be provided at the same time. Magnitude and phase information between current and voltage is collected. This information can be collected by, for example, a network analyzer. Typically, the signal(s) used are coherent signal(s) such that phase information can be readily extracted. Either a voltage source can be provided and current sensed, or a current source can be provided and voltage sensed. The process advances from the state 1710 to the state 1720.

In the state 1720, the process determines one or more relative relationship(s) between the electrical characteristics determined in the state 1720 for the first frequency and the second frequency. For example, rather than using impedance or admittance directly to distinguish tissue, the process can compute a relative relationship such as a ratio or difference between the characteristics measured at the first frequency and the second frequency, and then use the relative relationship(s) to distinguish types of tissue.

In the illustrated embodiment, the process computes the magnitude ratio |Y| 1722, the phase ratio θᵣ 1724, and the phase difference θ_{d} 1726, as described earlier in connection with FIGS. 10, 11, and 12, respectively. The process advances from the state 1720 to the decision block 1730.

In the decision block 1730, the process compares the one or more relative relationships to one or more thresholds representing criteria for the particular tissue that is being sought. These thresholds can be multi-dimensional as described in connection with FIGS. 13, 14, and 15 and will typically vary depending on the type of tissue to be detected. Experimental data can be used to determine appropriate thresholds for various tissue types. Such thresholds do not have to be linear and can be complexly shaped. In one embodiment, the thresholds are retrieved from a lookup table.

When the one or more relative relationships match with the criteria for the particular tissue, the process proceeds from the decision block 1730 to activate 1740 an indicator for the operator. The indicator can be, for example, a visual indicator such as a lamp, display device, or LED turning on, changing color, or otherwise visibly changing state, an audible indicator such as a buzzer, a vibrating indicator, or a combination of the same. The process is typically iterated repeatedly, and the particular iteration then ends and the process returns to the start of the loop to initiate a new iteration of the process.

When the one or more relative relationships do not match with the criteria for the particular tissue, the process then ends for the particular iteration and then returns to the beginning of the loop to begin a new iteration of the process.

### EXAMPLES

Table I illustrates applications of the method for identifying and accessing ducts to the identification of ductal orifices of several patients. The N=? column refers to the number of contact points of electrode on a nipple surface.

TABLE I

| human/nipple | | | impedance | capacitance | e.g.: ducts | guidewire |
|---|---|---|---|---|---|---|
| (right or left) | N = ? | potential | range | range | located | Photo? |
| A right | 1000 | 2 kHz | 2 kilohms to | not taken | 10 | no |
| | | | 18 megohms | | | |
| A left | 1000 | 2 kHz | 2 kilohms to | not taken | 8 | no |
| | | | 18 megohms | | | |
| B right | 100 | 5 kHz | not taken | 1 nanofarad to | 6 | yes |
| | | | | 500 picofarad | | |
| B left | 100 | 5 kHz | not taken | 1 nanofarad to | 7 | yes |
| | | | | 500 picofarad | | |
| C right | 200 | 3 kHz | 2 kilohms to | not taken | 12 | yes |
| | | | 30 megohms | | | |
| C left | 200 | 3 kHz | 2 kilohms to | not taken | 9 | yes |
| | | | 30 megohms | | | |
| B right | 10,000 | 7 kHz | 2 kilohms to | 1 nanofarad to | 7 | yes |
| | | | 10 megohms | 500 picofarad | | |
| D left | 10,000 | 7 kHz | 2 kilohms to | 1 nanofarad to | 9 | yes |
| | | | 10 megohms | 500 picofarad | | |
| E right | 10 | 10 kHz | 10 kilohms to | not taken | 4 | yes |
| | | | 200 kilohms | | | |
| E left | 10 | 10 kHz | 10 kilohms to | not taken | 6 | yes |
| | | | 200 kilohms | | | |

### Example 1

### Electrical Map of the Nipple Surface

A human nipple is dekeratinized and coated with a liquid crystal gel capable of registering a characteristic electrical signal, and a screen (a nylon wire matrix having tiny squares formed of the wire cross-hatch grid). The screen having a nylon wire grid is the size of the area of the nipple and is placed on the nipple. A reference electrode is placed at the base of the breast, and a test electrode is placed on the surface of the nipple. A potential and/or current is generated through the breast, and an electrical map is created by the variation of characteristic electrical signal values on the surface of the nipple. The readings of characteristic electrical signal measured in ohms by the machine capable of generating an electrical potential or current in an electrical circuit connected to the reference and test electrodes indicate likely locations of a ductal orifice with reference to a reference value (e.g., a value below which or above which a ductal orifice is indicated). An electronic picture of the nipple surface is retrieved from a machine for analyzing the electrical signal connected to the machine capable of generating an electrical potential or current in an electrical circuit. The machine for analyzing the electrical signal captures the electronic data comprising characteristic electrical signal values and analyzes it in a computer program, which data can be saved to a hard drive for later use. Marker tubes are placed in the regions of characteristic electrical signal indicating a ductal orifice through the fine holes of the screen. Once the entire nipple has a marker tube placed in all regions of characteristic electrical signal, i.e., the ductal orifices, the screen is removed. The marker tube-marked nipple is photographed for the patient's records. The entire process is repeated for the left nipple.

### Example 2

### Characteristic Electrical Signal Measurements in Rabbit and Human Breasts

Electrical measurements of impedance and/or capacitance were made using platinum or silver-silver chloride electrodes having a tip of between 1 μm and 1 mm. The electrode was manually placed at different locations on the surface of a nipple on a dead rabbit pelt (n=100 locations), the surface of a live rabbit nipple (n=90 locations), a masticated detached human breast (n=130 locations), and a human breast (n=70 locations). Measurements made on the surface of the human nipple at frequencies between 10 Hz and 100 kHz showed differences between the milk duct and the surrounding non-duct area of the nipple by detection of impedance measurements (as a characteristic electrical signal) of between 2 kilohms and 18 megohms and higher with some characteristic electrical signals too high to register. Differences in capacitance varied between the milk duct and the surrounding non-duct area of the nipple with values ranging from 1 nanofarad to 500 picofarad. Low impedance indicated a location of a ductal orifice that was verified by accessing an orifice at that location with a catheter. Locations that can be accessed by a catheter were registered as positive identification of a ductal orifice.

### Example 3

### Characteristic Electrical Signal Measurements Using a Bundle of 6 Electrodes on Masticated Nipple Surface

A bundle of electrodes was constructed having 6 electrodes in the bundle. Galactography needles (available from Manan, Inc. located at Gainsburg, Fla.) were cut to 3.5 cm. Cut needles were each inserted into glass capillary tubes (one needle per tube) such that 1 mm of needle protruded out of the bottom of the tube. Six capillary tubes having needles were bundled together. Each needle was connected to a lead wire at the top of the capillary tube, and the lead was connected to an LCR meter. A reference electrode was placed on the areola of a masticated breast. A potential was generated in each of the 6 electrodes (needles) at 36 different spots on the masticated breast nipple surface, i.e., the bundle was moved to 6 different locations or spots on the nipple surface. Potentials were generated at 1 kilohertz and 10 kilohertz at each of the 6 spots where the bundle rested. Impedance measurements taken at 1 kilohertz ranged from 20 kilohms to 1 megohm; impedance measurements taken at 10 kHz ranged from 10 kilohms to 144 kilohms as shown below in Table II.

TABLE II
Results of Bundle Electrode Test of Mastected Breast Nipple Surface

| | Frequency | | |
|---|---|---|---|
| spot/electrode | 1 kHz | 10 kHz | conclusions |
| spot1/electrode 1 | 71 kohms | 71 kohms | |
| 1/2 | 142 | 144 | based on both |
| | | | measurements (1 and |
| | | | 10 kHz) high impedance; |
| | | | possible duct |
| 1/3 | 29 | 27 | |
| 1/4 | 29 | 26 | |
| 1/5 | 20 | 19 | |
| 1/6 | 29 | 28 | |
| spot 2/electrode 1 | 28 | 13 | |
| 2/2 | 29 | 18 | |
| 2/3 | 100 | 22 | based on measurement |
| | | | at 1 kHz, high impedance; |
| | | | possible duct |
| 2/4 | 30 | 13 | |
| 2/5 | 25 | 11 | based on measurement |
| | | | at 10 kHz, low impedance; |
| | | | possible duct |
| 2/6 | 23 | 14 | |
| spot 3/electrode 1 | 29 | 20 | |
| 3/2 | 243 | 56 | based on measurement |
| | | | at 1 kHz, high impedance; |
| | | | possible duct |
| 3/3 | 208 | 52 | based on measurement |
| | | | at 1 kHz, high impedance; |
| | | | possible duct |
| 3/4 | 186 | 52 | |
| 3/5 | 124 | 30 | |
| 3/6 | 61 | 22 | |
| spot 4/electrode 1 | 22 | 17 | |
| 4/2 | 153 | 150 | based on both |
| | | | measurements (1 and |
| | | | 10 kHz) high impedance; |
| | | | possible duct |
| 4/3 | 46 | 13 | |
| 4/4 | 63 | 18 | |
| 4/5 | 80 | 27 | |
| 4/6 | 40 | 18 | |
| spot 5/electrode 1 | 85 | not taken | |
| 5/2 | 69 | not taken | |
| 5/3 | 11 | not taken | |
| 5/4 | 215 | not taken | |
| 5/5 | 308 | not taken | |
| 5/6 | 1000 | not taken | Based on measurement taken |
| | | | at 1 kHz; high impedance; |
| | | | possible duct |
| spot 6/electrode 1 | 128 | 32 | |
| 6/2 | 82 | 29 | |
| 6/3 | 520 | 69 | based on measurements taken |
| | | | at 1 kHz; high impedance; |
| | | | possible duct |
| 6/4 | 215 | 26 | |
| 6/5 | 33 | 19 | |
| 6/6 | 32 | 15 | |

Furthermore, the following prophylaxis and treatment examples make use of the rat mammary tumor model, which has been deemed an appropriate experimental model for understanding breast cancer in humans (Sukumar et al., Mutation Res. 333 (1-2): 37-44 (1995); Russo et al., supra). In fact, 90-100% of female rats develop mammary tumors in this model when they are administered the carcinogen NMU at 55 days of age (Sukumar, Cancer Cells 4:199-204 (1990)).

### Example 4

This example demonstrates the successful delivery of virus and other agents into the mammary ductile tree by a single injection through the teat.

ADV/CMV-β-gal (from Dr. William Burns, Johns Hopkins University) is an adenoviral 5 vector constructed with a β-galactosidase gene controlled by a cytomegaloviral promoter. It was delivered into the mammary gland by injection of a viral suspension in 20 μl of 0.2% trypan blue in Tris buffer through the teat of a rat. The nipple was extruded, and the sphincter removed by excising the nipple. In the rat, the muscle prevents fluid from regurgitating into the breast and had to be excised in order to visualize the ductal opening and administer the agent. Trypan blue was used as a tracking dye to ensure correct delivery to the ductile tree. Injection about 30 days postpartum resulted in the mammary epithelial tree being clearly visible. Ethyl alcohol (70%) was also successfully delivered by a single injection through the teat.

### Example 5

This example demonstrates that adenovirus can efficiently transduce human mammary epithelial cells in vitro.

ADV/CMV-β-gal was used to transduce HBL100 mammary epithelial cells in vitro. The β-gal enzyme in this construct contains a nuclear localization signal and results in dense nuclear staining. HBL100 cells (10²) were plated in 24-well plates, and transduced with virus at various doses and stained with X-gal 48 hrs later. Essentially all cells were infected at a moi=10⁴.

This experiment also has been performed in human mammary tumor cells MCF-7 (American Type Culture Collection (ATCC), Rockville, Md.), human mammary epithelial cells MCF-10A (ATCC), and two rat mammary cancer cell lines, RBA (from Dr. Leonard Cohen) and 37-2 (from Dr. C. Marcelo Aldaz) with the same results. More efficient adenoviral constructs have been used, thereby achieving 100% infection at a moi=10³. These experiments demonstrate successful infection by and expression of adenovirus carrying the lacZ indicator gene, which permits staining the cells blue by the expression of the enzyme β-galactosidase.

### Example 6

This example demonstrates that infection with an AdHS-tk construct followed by GCV treatment effectively kills mammary tumor cells in vitro.

RBA and NMU68 are two rat mammary tumor cell lines derived from a DMBA- and a NMU-induced tumor, respectively [DMBA=dimethylbenz[a]anthracene, NMU=N′-nitro N′-methylurea]. Each cell line was plated at a density of 5×10² in 48-well plates (1.1 cm) and allowed to settle overnight. The next morning, they were transduced with AdHS-tk (Chen et al., PNAS(USA) 91:3054-57 (1994); obtained from S. Woo and E. Aguilar-Cordova, Baylor College of Medicine, Houston, Tex.) at titers of 0, 100, 500, and 1000 moi, and then, 6 hrs later, GCV (10 μg/ml) was added to the culture media. The cells were maintained in the presence of GCV for 3 days and then counted using trypan blue exclusion as a measure of cell viability. The cell numbers were normalized to the growth of cells in the absence of GCV. The results are shown in FIG. 1. More than 80% of the cells of each cell line were killed at a moi of 10³.

### Example 7

This example demonstrates the prophylactic effect of the method of the invention.

The mammary glands (6 on each side) of six virgin 50 day old Sprague Dawley rats were injected with AdHS-tk on the left side and trypan blue on the right side or left untreated. One rat remained completely untreated with the virus or GCV and served as a positive control for NMU-induced tumorigenesis. On the day of surgery, rats were given an intramuscular injection of 5 μg estradiol valerate and were anesthetized with an isofluorane/O₂ mixture. The nipples were cannulated with a 33 gauge needle. Twenty μl of AdHS-tk diluted in trypan blue carrier (1 mM MgCl₂, 20 μg/ml polybrene in 10% glycerol, 0.4% trypan blue in saline) at a concentration of 5×10⁷ particles/μl were injected into the duct. Carrier control mammary glands received 20 μl of trypan blue carrier alone. An animal was considered treated when at least three glands were successfully injected with trypan blue and another three glands were successfully injected with AdHS-tk. The remaining glands were left untreated. Twelve hrs later, the rats were injected with 125 mg/kg body weight GCV twice daily for three days. The rats were then given a second intramuscular injection of 5 μg estradiol valerate and intraperitoneal injections of 100 μg/kg body weight GCV once daily for three days. Five to seven days after GCV treatment, the rats were given an intravenous injection of NMU dissolved in 0.05% acetic acid (Ash Stevens, CO; 50 mg NMU/kg body weight) and were subsequently monitored for general health and the appearance of tumors at weekly intervals for 8 months. The results were as shown in Table III below:

TABLE III

| | | No. of Total | % of Glands |
|---|---|---|---|
| Treatment | No. of Tumors | Glands | with Tumor |
| Untreated | 5 | 12 | 41.7 |
| Control | | | |
| Trypan Blue | 5 | 17 | 29.4 |
| And Ganciclovir | | | |
| No injection; | 5 | 20 | 25.0 |
| Ganciclovir | | | |
| AdHS-tk and | 2* | 35 | 5.7 |
| Ganciclovir | | | |

*The two tumors (size <5 mm) in this group were detected during the necropsy at the termination of the experiment 8 months later. The difference between tumors appearing in treated versus control glands was significant by Chi² analysis (p < 0.01).

The above results show that the method of the invention inhibits the formation of cancer of ductal epithelial origin in this rat model, in which NMU induces the formation of mammary tumors in 90-100% of female rats of similar age. Surprisingly and unexpectedly, this prophylactic effect was achieved without extensive destruction of the mammary glands. These data demonstrate that selective destruction of epithelial cells, e.g., key stem cells, can be sufficient to provide prophylactic protection against carcinogen-induced tumor formation in the ductal epithelium of the mammary gland.

### Example 8

This example demonstrates the efficient transfection of mammary epithelial cells in vivo.

Lytic Vaccinia virus (10⁶ Vaccinia-HA, which also carries lacZ, in 20 μl 0.2% trypan blue) was injected into the mammary glands through the teat of 45 day old virgin rats. Contralateral control glands were injected with 0.2% trypan blue. The glands were excised after 3 days. Frozen mammary gland sections were stained with X-gal and counterstained with eosin. When Vaccinia-HA was injected via the rat teat, it was able to infect the epithelial cells. At 3 days post-infection, the X-gal staining was confined primarily to the epithelial cells.

### Example 9

This example demonstrates the cytotoxicity of Vaccinia/HA on HBL100 cells in vitro.

HBL100 cells (from ATCC, Rockville, Md.) were plated in DME:F12 medium (50% Dulbecco's Modified Eagles Medium: 50% Ham's F12 Supplement) containing 10% fetal bovine serum and 10 μg/ml insulin at a density of 5×10⁴ cells/well and incubated at 37° C. overnight. Vaccinia/HA, at concentrations of 0 moi, 0.1 moi, or 1.0 moi, was added to the culture medium, and the cells were incubated at 37° C. for at least 3 days. More than 90% of the cells were dead within 72 hrs of infection at 0.1 moi.

### Example 10

This example shows the death of rat mammary tumor cells in culture by infection with Vaccinia/HA.

Cells of rat mammary cancer cell line RBA were plated in growth medium at a density of 5×10⁴ cells/well and incubated at 37° C. overnight. Vaccinia virus engineered to express β-galactosidase and hemagglutinin genes (Vaccinia/HA) was added to the culture medium at concentrations of 0 moi, 0.1 moi, or 1.0 moi and incubated at 37° C. for at least 3 days. Up to 90% of the cells were lysed within 72 hours of injection at 1.0 moi.

### Example 11

This example demonstrates the destruction of mammary epithelium by transfection with Vaccinia/HA in vivo.

The mammary glands of 45-day old virgin rats were injected through the teat with 1×10⁷ particles of Vaccinia/HA in 20 μl 0.2% trypan blue (tracking dye). Contralateral control glands were injected with 0.2% trypan blue. The glands were excised after 3 days, fixed in chloroform:methanol:acetic acid and stained in iron-hematoxylin. Branching structures of a whole-mounted mammary gland injected with tracking dye alone were visible up to the end buds and alveoli. Also visible as brown bodies were the mammary lymph nodes. Examination of a whole-mounted mammary gland of the same rat receiving Vaccinia/HA in trypan blue on the contralateral side revealed that only about 30% of the ducts remained. In addition, the lymph nodes were considerably enlarged, denoting the mounting of an immune response to clear the Vaccinia from the vicinity.

### Example 12

This study evaluated the efficacy of five test articles (four marketed anticancer agents), administered directly into the mammary gland ducts via teat infusion, for therapy and prevention of ductal carcinoma in situ in accordance with the preferred aspects of the present invention.

The objective of this study was to evaluate the efficacy of five test articles (four marketed anticancer agents), administered directly into the mammary gland ducts via teat infusion, for therapy and prevention of ductal carcinoma in situ in an N-Nitroso-N-Methylurea (MNU)-induced tumor formation rat model. Based upon previous animal studies, candidates were selected for more rigorous evaluation at B Braun Biological Test Center (BTC) in Irvine, Calif. This facility was chosen to continue the preclinical work, started by Dr. Sukumar at JHU in order to support studies with a larger number of animals, in compliance with Good Laboratory Practices.

Methods: The cytotoxic agents included carboplatin (tested at two dose levels), Taxol® (paclitaxel), Herceptin® (trastuzumab), and Doxil® (doxorubicin HCl). The agents were tested in 120 female Sprague Dawley rats (n=20 per treatment). On Day 1, each rat was injected intraperitoneally with MNU to induce tumor formation. On Day 15, each rat received one of the following six intra-ductal treatments: No treatment (control); infusion of carboplatin (0.75 mg/teat); infusion of Taxol® (0.15 mg/teat); infusion of carboplatin (0.42 mg/teat); infusion of Herceptin® (1.67 mg/teat); or infusion of Doxil® (0.15 mg/teat). Each rat was dosed in 11-12 teats except for one rat with 5 non-infusable teats. Rats were weighed prior to MNU injection, prior to intra-ductal infusion, and once weekly throughout the study. Dose levels are presented in Table IV below:

TABLE IV

| | | Test Article Administration, Day 15 (Intraductal Administration to 12 Teats) | | | | | | |
|---|---|---|---|---|---|---|---|---|
| | | | Clinical | Rat BSA | Total | Dose | Dose | Dose |
| | | | Dose | (m2) | Dose | Concentration | Volume | Level |
| Group | No. | Test Article | (mg/m2) | (BW = 100 g) | (mg/rat) | (mg/mL) | (μL/teat) | (mg/teat) |
| A | 20 | N/A | N/A | N/A | N/A | N/A | N/A | N/A |
| B | 20 | Carboplatin | 450 | 0.02 | 9 | 10 | 75 | 0.75 |
| C | 20 | Taxol ® | 90 | 0.02 | 1.8 | 2 | 75 | 0.15 |
| D | 20 | Carboplatin | 250 | 0.02 | 5 | 5.56 | 75 | 0.42 |
| E | 20 | Herceptin ® | 1000 | 0.02 | 20 | 22.2 | 75 | 1.67 |
| F | 20 | Doxil ® | 90 | 0.02 | 1.8 | 2 | 75 | 0.15 |

Rats were observed twice daily for mortality/morbidity and 1-3 times weekly for general appearance, stool appearance, signs of toxicity, and palpable mass formation until death or euthanasia. Necropsy was originally scheduled for Day 120±2 (approximately 17 weeks). Per amendment, necropsy was re-scheduled for Day 180±2 (approximately 26 weeks) for all groups except the Taxol® and Herceptin® groups, which were necropsied on Day 120±2. At necropsy, organs and tissues were grossly observed, and the entire bilateral mammary gland system, visible masses, and abnormal tissues were collected and retained in formalin.

Results: Three rats died and 15 rats were euthanized due to MNU treatment and subsequent tumor formation with adverse sequelae prior to scheduled euthanasia.

Within each treatment group, palpable masses were first detected in a few rats during Weeks 5-9. During the course of the study, the incidence of palpable masses was generally highest and most rapid in control rats and rats dosed with Taxol®, and it was lowest and least rapid in rats dosed with carboplatin (0.75 mg/teat) and Doxil®. At final observations prior to death or euthanasia, the number of rats with masses was highest in the control group (18 of 19 rats) and Taxol® group (20 of 20 rats), and it was lowest in the Doxil® group (9 of 20 rats) and carboplatin (0.75 mg/teat) group (11 of 20 rats).

During necropsy, visible masses were often characterized as vascularized or semi-vascularized, lobular or multilobular, soft or firm/solid, and abnormally colored (pale/white, yellowish, greenish, or dark). Masses ranged in size from less than 0.5 mm to more than 5 cm. Most masses were located subcutaneously in the axillary, inguinal/abdominal, perianal, and neck areas, although some were found among the internal organs.

Among rats that were necropsied, no visible masses or mammary lesions were found in 25 rats as follows: One of 19 control rats; 7 of 19 rats dosed with carboplatin (0.75 mg/teat); 5 of 20 rats dosed with carboplatin (0.42 mg/teat); 4 of 20 rats dosed with Herceptin® (1.67 mg/teat); and 8 of 20 rats dosed with Doxil® (0.15 mg/teat). Visible masses were found in all 20 rats dosed with Taxol® (0.15 mg/teat); these rats were all necropsied 4 months post-MNU injection.

The most common findings of internal organs (excluding masses) were an enlarged or discolored spleen (13 rats), an enlarged or discolored liver or attached liver-lobes (6 rats), enlarged, discolored, or fluid-filled ovaries (5 rats), fluid-filled or discolored uterine horns (4 rats), and kidney anomalies (2 rats).

Conclusions: Intraductal infusion with Doxil® (0.15 mg/teat) or carboplatin (0.75 mg/teat) was associated with the lowest incidence of tumor mass formation within 6 months of MNU-injection in female rats, while infusion with Taxol® (0.15 mg/teat) was associated with the highest incidence of mass formation.

### Example 13

This study demonstrates that the ductal orifice can be located, cannulated and cytotoxic drugs can be easily administered into breast ducts with minimal toxicity using a device in accordance with the preferred aspects of the present invention—as exemplified in FIGS. 9-17.

Intraductal administration of cytotoxic agents was shown to inhibit the development of breast cancer in Her-2/neu over-expressing mouse and MNU rat models (Murata 2006). This dose escalation study was performed to demonstrate the safety of this approach in women prior to mastectomy. The study was performed in Cancer Hospital, Chinese Academy of Medical Sciences, Beijing, China where the standard of care includes a long preoperative hospital stay prior to mastectomy. Two drugs, pegylated liposomal doxorubicin (PLD) and carboplatin (C) were administered at 3 dose levels (PLD: 10, 20, 50 mg and carboplatin 60, 120, 300 mg) with the highest dose approximating the clinical intravenous dose. There were five subjects in each group with 15 subjects treated with each drug once. After informed consent, under local anesthesia subjects underwent cannulation of 5-8 ducts with intraductal instillation of the drug. Venous blood samples were obtained for pharmacokinetic analysis. The total dosage was divided by the number of cannulated ducts to yield a dose per duct. The breast was removed surgically as planned 2-5 days post treatment and the treated ducts were marked to enable identification on pathological evaluation. Intraductal administration was generally well-tolerated with mild, transient breast discomfort upon administration associated with the rate of infusion. Clinically significant laboratory adverse events were limited to decreases in hemoglobin following mastectomy, consistent with blood loss. Neither leucopenia nor thrombocytopenia were observed in the study. In the carboplatin arm, three women at the 300 mg dose experienced mild nausea and vomiting. In the PLD arm most women had mild erythema and swelling of the breast over the 72 hours following the drug administration while the women receiving the highest dose experienced local erythema until the time of surgery. Pharmacokinetic analysis showed that carboplatin rapidly entered systemic circulation with an early peak time (tmax˜30 min) with a corresponding plasma ultrafiltrate AUC (area under the curve) consistent with the Calvert Formula using estimated GFR. Total plasma doxorubicin had delayed peak concentration times (tmax>48 hours) with a linear dose response and peak concentrations substantially lower than expected from equivalent IV dosing. No doxorubicinol metabolite was detected in the plasma. Pathological examination showed the drugs were widely distributed throughout the ductal systems reaching terminal duct lobular units, and there was a significant although variable dose-related epithelial cell loss in ducts with dye indicating drug effect.

### Example 14

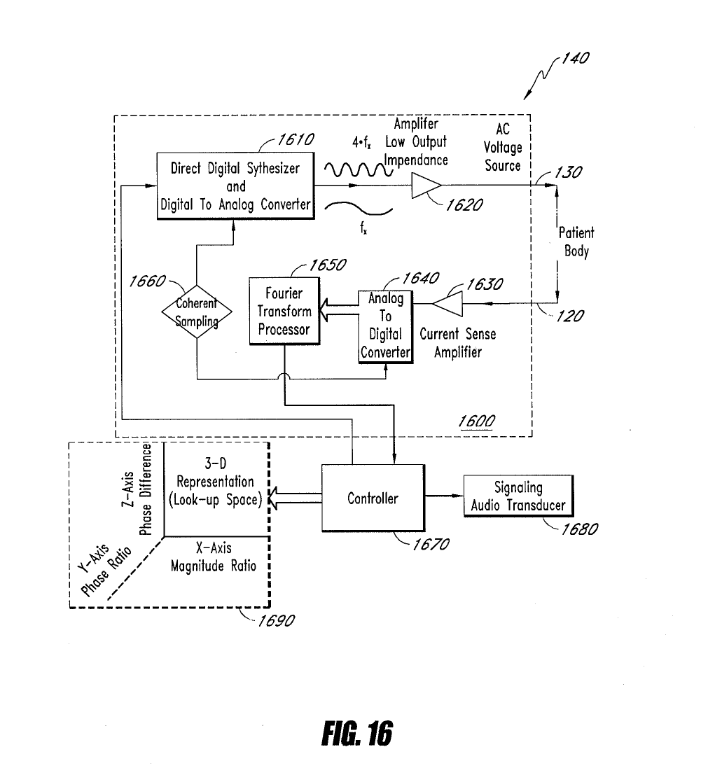

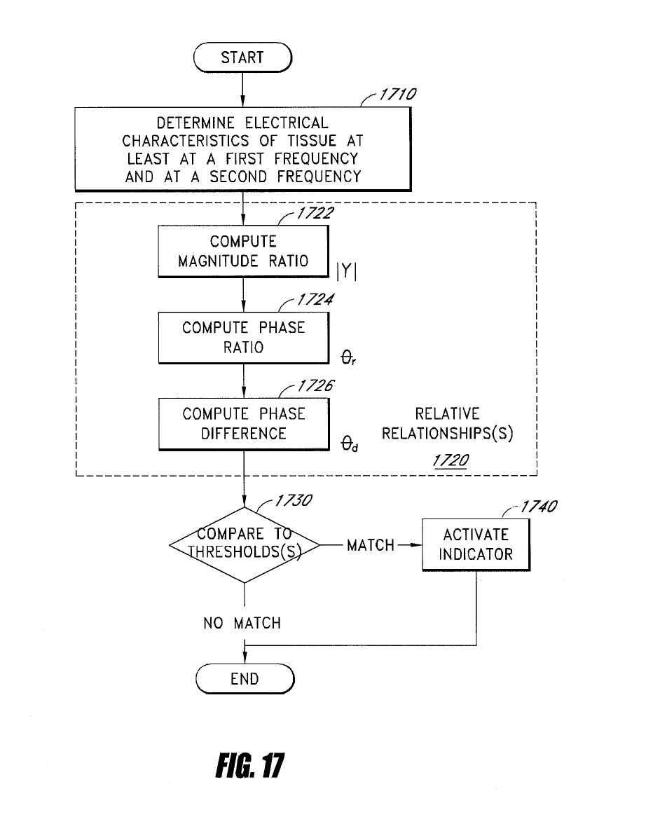

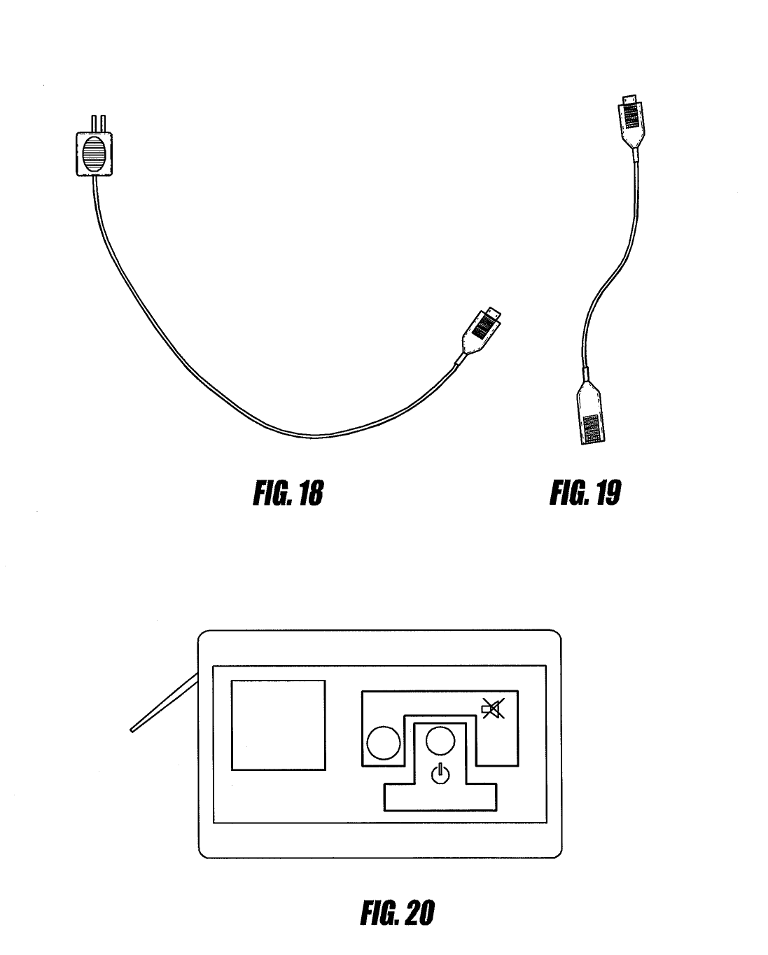

This study compares clinical indices with regard to prior art devices and a device in accordance with preferred aspects of the present invention (in this Example, “the Study Device”; more particular disclosure of the Study Device in accordance with an embodiment of the present invention. The study device is pictured in FIGS. 18-20 and included a probe-catheter with insertion catheter/probe, hub, hub anchor, fluid connecting tube, sensing cable, extension cable, electrode pad, electrode cable, electronic sensing unit (ESU), as described below.

Insertion Catheter-Probe.

The Insertion Catheter/Probe (ICP) is the working tip of the catheter and is designed to cannulate an individual mammary duct to deliver fluid without causing perforation of the mammary duct wall. It is a 7 inch long, thin-walled polymer tube over a stainless steel wire with a stainless steel ball at the tip of the catheter. The insertion catheter also acts as a sensing probe to detect the mammary duct openings on the nipple. The thin-walled clear polymer tube is designed to carry fluid from the Hub into the mammary ducts.

Hub.

The Hub is a single piece of molded plastic with 3 openings: one opening holds the ICP and the other two hold the Fluid Connecting Tube and the Sensing Cable.

Hub Anchor.

The Hub Anchor is a small circular adhesive pad used to anchor the Hub to the patient to avoid dislodging the device.

Fluid Connecting Tube.

The clear Fluid Connecting Tube carries fluid from the syringe to the Hub. It is made from a translucent polymer allowing fluid visualization. It has a standard Luer-Iock fitting on the end to allow it to connect to commercially available syringes.

Sensing Cable.

The white Sensing Cable carries an electrical signal from the stainless ball on the end of the ICP to the Electronic Sensing Unit. It is permanently affixed to the Hub and attaches to the Electronic Sensing Unit with a connector.

Electrode Pad.

The Electrode Pad is a conductive pad which connects to the Electrode Cable. The pad adheres to the skin of the patient and has a snap to connect to the Electrode Cable.

Electrode Cable.

The Electrode Cable is a connecting cable which attaches to the Electrode Pad and delivers a signal from the Electronic Sensing Unit. It attaches to the Electronic Sensing Unit using a connector.

Electronic Sensing Unit (ESU).

The Electronic Sensing Unit (ESU) aids the user in locating milk duct openings.

The ESU is composed of a battery-powered electronics circuit, a tab to turn the device on, two light emitting diodes (LED) to indicate power and sound respectively, a speaker which emits an audible signal to indicate a likely mammary duct, and a plastic enclosure.

The ESU delivers a low-voltage, high-frequency electrical signal to the patient through the Electrode Pad via the Electrode Cable. The ESU analyzes the level and characteristics of this signal at the nipple surface where the end of the Insertion Probe Catheter (ICP) is placed. The larger multi-colored LED indicates the status of the device, including when it is on and ready to use and when the device is in fault and unusable. The audible signal alerts the user that the probe is directly over a possible mammary duct opening. The sound will continue until the “sound off” button is pressed, the LACD is removed from the opening on the nipple or the LACD is removed from the ESU and replaced with a new one. The smaller LED located to the right of the status LED indicates when the device is in the “sound off” mode. When the “sound off” is selected the LED will turn yellow.

The battery life of the ESU is approximately 2 hours. After 1 and ˜hours, the larger status LED light will display a blinking green light indicating a low battery. The user has approximately an additional ˜hour to complete the procedure or replace the ESU with a new one.

Operation of the test device was in accordance with the following instructions:
The WHM Intraductal Catheter System (ICS) is a device designed to aid the user in identifying ductal orifices for subsequent cannulation and fluid instillation.
The Electronic Sensing Unit (ESU) of the ICS is intended to allow the user to more quickly and easily identify ductal orifices by guiding the user to likely areas on the nipple surface. The device does this by analyzing the electrical characteristic of the nipple. It is not intended to be able to definitively locate ductal orifices, some areas that it identifies as likely areas may not turn out to have ductal orifices and there may be other areas on the nipple surface not identified that do have ductal orifices. It is a useful aid that has been shown to simplify the ductal orifice cannulation procedure. The ESU utilizes the tip of the Locator and Catheter Device (LACD) as its probe. Furthermore, the system does not use dilators like those included with commercially available galactography kits or ductal lavage kits. This allows the user to immediately attempt to cannulate any area on the nipple that the user identifies as a likely ductal orifice without having to switch to another device. The user merely inserts the LACD into the possible ductal orifice and, if successful, the LACD becomes the catheter for fluid instillation.
9 LACD devices are included in each ICS kit which means there should be at least one LACD per ductal orifice. It is possible, but generally unnecessary, to use a single LACD in more than one ductal orifice of the same patient. The ESU has a working time of approximately 2 hours and once turned on cannot be turned off. All patient contact devices in the kit are sterile and disposable and cannot be re-sterilized or re-used in another treatment session.
   1. When the user is ready to begin identifying ducts, pull out the green plastic tab located near the side ports on the ESU. This will turn on the device. Upon start-up the device will go thru a self-test sequence. During this sequence, the power LED will initially be red then turn a solid green indicating the device is fully operational. If, instead of a solid green light, the power LED blinks green or remains red, this indicates a low battery or faulty device, respectively. In this circumstance, replace the ESU device with a new unused device. The sound status LED will also go thru a start-up sequence, blinking yellow and then turning off. If the blinking yellow LED does not turn off, the device is faulty and should be replaced with a new unused device. The device will also make a short beep on start-up to confirm the speaker is functional. If the device does not sound a short beep upon start-up depress the “sound off” button twice on the front of the ESU. If no sound is heard on upon the second depression of the “sound off” button, the device is faulty and should be replaced with a new unused device.
NOTE: When the sound status LED demonstrates no color, this indicates the sound for the device is on. When the sound status LED is yellow, this indicates the sound has been muted.
   2. If appropriate, apply the sterile Protective Sleeve to the ESU.
   3. The Electronic Sensing Unit (ESU) should be placed near the patient's breast/chest wall in order to attach the Electrode Cable and Sensing Cable for the procedure.
   4. The Electrode Pad should be placed adjacent to the areola of the breast undergoing the procedure. Cleanse the area for Electrode Pad placement with alcohol or a suitable disinfectant. Allow disinfectant to dry and remove adhesive backing cover from the Electrode Pad and attach the pad to the skin of the breast within approximately 2 inches of the areola. Subsequently, snaphthe sterile Electrode Cable to the Electrode Pad.
   5. Insert the male end of the Electrode Cable into the green labeled side port on the ESU.
   6. If necessary the sterile Extension Cable can be used to provide more length to the Sensing Cable and to avoid having to connect and disconnect the Sensing Cable from the ESU each time a new LACD is used. Insert the Extension Cable into the white labeled side port on the ESU.
   7. Determine the proper nipple and/or breast anesthetic that be may applied prior to or during the procedure. Apply anesthetic prior to locating orifices or inserting the Probe-Catheter.
   8. Once adequate anesthesia has been applied and the patient is in a supine position, don sterile gloves. Wipe the nipple/areolar complex with a suitable disinfectant and cover the breast with a sterile drape.
   9. Prepare the first sterile Locator and Catheter Device (LACD). Insert the male end of the white Sensing Cable into the white labeled side port on the ESU or to the Extension Cable. Attach a sterile 5 cc syringe containing 3-5 cc of sterile saline or solution of 1% lidocaine without epinephrine to the Fluid Connecting Tube.
Note: If patient is allergic to lidocaine, determine another anesthetic to utilize or prime tubing with sterile fluid.
   10. Prime the tubing with enough lidocaine to clear the air from the tubing. Remove protective sheet from LACD.
   11. Dry the nipple with sterile gauze and examine it to ascertain potential ductal orifices. These may be evident with creases, folds, openings on the top of the nipple. Probe these openings with the tip of the LACD to further determine if they are likely ductal orifices. If an area is likely to be near a ductal orifice the ESU will emit an audible tone. At this point, if it is desired, the sound being emitted from the ESU can be turned off by pressing the “sound off” button on the front of the ESU. When the device is muted the sound status LED will be illuminated yellow.
   12. Upon the identification of the ductal orifice either visually or with the assistance of the ESU, attempt to insert the Probe-Catheter tip of the LACD into the duct approximately 1-2 cm. The ProbeCatheter tip has a dark mark starting at 1 cm and continuing to 2 cm. This is a visual guide to aide the user in depth of insertion. If the Probe-Catheter tip will not insert into the ductal orifice, try to insert it at an oblique angle. If the Probe-Catheter tip enters the ductal orifice but there is some resistance before it reaches 1 cm, lift the nipple two to three times its normal height. If resistance is still met, attempt to instill fluid. If the tip cannot be inserted, resistance is still met, or fluid cannot be instilled, do not force the LACD into the opening. Remove the LACD and attempt to identify another opening.
   13. Once, the LACD is inserted into the duct opening, remove the protective covering from the Hub Anchor and attach it to the breast/chest wall of the patient. Slowly (approximately 0.50 cc per 30 seconds) instill 0.5 cc of sterile saline or 1% lidocaine into the orifice. If fluid cannot be instilled lift the nipple to two to three times its normal height and attempt to instill fluid again. If fluid still cannot be instilled, remove the LACD and attempt to identify another opening.
   14. Detach the Sensing Cable from the ESU. Further fluid instillation will occur after the remaining orifices have been identified.
   15. Prepare the next sterile LACD, by priming the Fluid Connecting Tube with sterile saline or 1% lidocaine and attach the male end of the Sensing Cable to the ESU or Extension Cable as in Step 9.
   16. Repeat steps 9-14 for as many orifices as desired with a new LACD for each orifice. There are approximately 5-9 ducts in the breast. The ICS includes a sufficient number of LACDs to cannulate all possible orifices, if desired.
NOTE: If the sound was turned off with the prior LACO, be sure to press the “sound off” button. A single noise will be emitted from the ESU indicating the Probe-Locator sound is no longer muted and yellow sound status LED will turn off. If this sound is not heard, or light does not turn off, press the button two more times.
   17. Once, all of the LACD devices are seated in the duct openings and sterile saline or 1% lidocaine has been instilled, detach the 5 cc syringe which contained the sterile saline or 1% lidocaine from each LACD device and replace it with the appropriate syringe containing the fluid to be instilled. This should be done for all LACD devices.
   18. With the fluid containing syringe attached, begin instilling the fluid into the duct through the Fluid Connecting Tube of the LACD. This should be done slowly (approximately 0.50 cc per 30 seconds). There may be some resistance or discomfort. If the patient feels discomfort, try to instill fluid at a slower rate. If the discomfort continues, wait 30 seconds to 1 minute to see if the discomfort ceases, if it does continue to instill fluid, if not cease instillation into that duct. FDAContinue with LACD devices inserted into duct openings. Care should be taken not to attempt to fully infuse a single duct twice in the same session.
   19. Upon completion of fluid instillation in the desired duct(s) the LACD devices and electrode pad can be removed from the patient. Discard the LACD and all associated Intraductal Catheter System devices appropriately.
To perform procedures on the opposite breast, a new Intraductal Catheter System will need to be used.

Thirty-two subjects were enrolled in the study and underwent the study procedures. There were 24 Caucasians, 6 Hispanics, 1 Asian, and 1 subject of East Indian descent. The median age was 46 years (range 23-74). The median age at menarche was 12.5 years (range 9-17) and median number of pregnancies was 1.5 (range 0-5) and the median number of live births was 0.5 (range 0-4). The median bra size was 34 (range 32-40) and median bra cup size was B (range A-DD). Fourteen subjects were post-menopausal and eighteen were pre-menopausal. As indicated in Listing 2, four subjects had prior history of excisional biopsies, two had prior core needle biopsies and one had a prior fine needle aspiration. No subjects had a history of mastitis, prior breast cancer, or lumpectomy/radiation.

All 32 of the subjects provided complete answers for the purposes of establishing eligibility; however, one subject failed to satisfy eligibility criteria due to breast implants. The subject was determined to have bilateral breast implants based on subject commentary during the procedure. A mammary duct in this subject was not cannulated by either device and the subject is excluded from all following statistical analyses and summary information. Thus there were 31 eligible subjects for the purpose of the analyses.

The protocol specified 3 cc of NS was to be infused into a duct to act as a contrast material for the purpose of confirming that ductal cannulation was successful via ultrasound. In practice approximately 1 cc of NS was infused during real time ultrasound, which was sufficient in all cases of ductal cannulation to verify that the cannulation was successful. Confirmation ultrasound images were obtained for all subjects.

Overall, the Study Device was significantly more successful in achieving ductal cannulation than was the other devices. Successful cannulation appeared to be more rapid with the Study Device with no apparent difference between the two approaches in overall pain or discomfort experienced by subjects undergoing successful cannulation. No single predicate device that combines the ability to identify and cannulate breast ducts is approved. The DucPrep™ Aspirator depends on the ability to elicit nipple aspirate fluid for duct identification. In this study, only three subjects were able to have nipple aspirate fluid elicited. These three subjects were successfully cannulated using the Pro•Duct catheter (9.7%). Ductal identification and cannulation were successful with the Study Device in 27 of 31 eligible subjects (87.1%). The median time to successful cannulation of a breast duct with the predicate devices appeared to be longer (8.3 minutes) relative to the Study Device (3.2 minutes). In general, there was only minor discomfort associated with the use of the Study Device or the Predicate Device which would be further reduced during typical practice with a nipple block. During this study, the typically available nipple block was not utilized to allow more direct pain comparisons.

There did not appear to be any correlation between success or failure with either technique and age, age at menarche, menopausal status, and number of live births, bra size, bra cup size or prior history of surgery. The failure to successfully cannulate the one subject with nipple inversion is consistent with the presence of underlying fibrosis (which can range from moderate to severe) in this condition. The investigator determined after one attempt that the nipple inversion was too high a grade to allow for successful cannulation.

All publications, patents, and patent applications cited herein are hereby incorporated by reference to the same extent as if each individual document were individually and specifically indicated to be incorporated by reference and were set forth in its entirety herein.

While this invention has been described with emphasis upon preferred embodiments, it will be obvious to those of ordinary skill in the art that the preferred embodiments may be varied. It is intended that the invention may be practiced otherwise than as specifically described herein.

### LIST OF REFERENCES

1. Wrensch M R, Petrakis N L, King E B, Milke R, Mason L, Chew K L, Lee M M, Ernster V L, Hilton J F, Schweitzer R, Goodson W H, and Hunt T K, (1991) Am. J. Epidemiology, v. 135 (2): 130-141.
2. King, E B, Chew K L, Petrakis N L, Ernster V L, (1983) JNCI 71 (6) 1115-1121.
3. Papanicolaou G N, Holmquist D G, Bader G M, Falk E A (1958) Cancer, 11:377-409.
4. Goodson W H & King E B, THE BREAST: Comprehensive Management of Benign and Malignant Diseases; Chapter 4: Discharges and Secretions of the Nipple volume 1; pp. 51-74 (2.sup.nd Ed. 1998; Eds. Kirby & Copeland, Philadelphia, Pa.)
5. Love & Barsky, (1996) Lancet 348: 997-999
6. Barsky & Love (1996) “Pathological analysis of breast duct endoscoped mastectomies” Laboratory Investigation, Modem Pathology, Abstract 67
7. Lewis (1997) Biophotonics International, pages 27-28, May/June 1997
8. Sartorious (1995) Breast Cancer Res. Treat. 35: 255-266
9. Sartorious (1987) Contrast Ductography for the recognition and localization of benign and malignant breast lesions: An improved technique” in Logan (ed.) Breast Carcinoma, New York, Wiley pp. 281-300
10. Petrakis (1993) Cancer Epidem. Biomarker Prev. 2:3-10
11. Petrakis (1986) Breast Cancer Res. Treat 8: 7-19
12. Wrensch et al (1992) Am. J. Epidem. 135:130-141
13. Wrensch et al (1990) Breast Cancer Res Treat 15: 39-21
14. Wrensch et al (1989) Cancer Res. 49:2168-2174
15. Makita et al (1991) Breast Cancer Res Treat 18: 179-188
16. Okazaki et al (1991) Jpn J. Clin. Oncol. 21:188-193
17. Diner et al (1981) American J. Radiology 137: 853
18. Tabar et al (1983) Radiology 149: 31
19. Threatt et al (1987) Ductography p. 119 Basset and Gold eds, Grune & Stratton, Orlando
20. Pansera F (1990) Medical Hypotheses 33: 107-111
21. Faupel and Hsu, (1996) Electropotentials in the clinical assessment of breast neoplasia, Dixon eds 37-44

What is claimed is:

1. A kit, comprising:
an analyzer configured to indicate a characteristic electrical value in an electrical circuit comprising a duct of a mammary gland;
a catheter operable to access the duct when the analyzer indicates the characteristic electrical value, wherein the catheter comprises an electrode operably coupled to the analyzer; and
a therapeutically or prophylactically effective amount of an agent in a form capable of introduction through the catheter.

2. The kit of claim 1, wherein the agent is effective to destroy at least a portion of the ductal epithelium.

3. A method of delivering material to a milk duct of a mammary gland, the method comprising:
passing an electrical current or potential through the mammary gland and adjacent tissue using a catheter having a lumen and distal end region comprising an electrode;
identifying a ductal orifice of the mammary gland based on measured values of the electrical current or potential;
introducing said catheter into said ductal orifice; and
delivering the material through the delivery lumen and into the milk duct.

4. The method of claim 3, wherein the material comprises at least one of a hydrogel, a nanocarrier, a nanogel particle, an aggregated nanogel particle, an imaging agent, and a therapeutic or prophylactic agent.

5. The method of claim 3, wherein the material comprises a crosslinked PEG.

6. The method of claim 3, wherein the material comprises an imaging agent associated with a hydrogel or a nanocarrier or a nanogel particle or an aggregated nanogel particle.

7. The method of claim 6, wherein the association is a covalent bond.

8. The method of claim 4, wherein the imaging agent is a dye or contrast agent.

9. The method of claim 4, wherein the imaging agent is visible by visual observation or by radiographic, MRI, PET, or SPEC examination.

10. The method of claim 4, wherein the therapeutic or prophylactic agent is an epithelial cell destroying material.

11. The method of claim 10, wherein the therapeutic or prophylactic agent is a chemotherapeutic material.

12. The method of claim 10, wherein the therapeutic or prophylactic agent is selected from the group consisting of genistein, okadaic acid, 1-β-D-arabinofuranosyl-cytosine, arabinofuranosyl-5-aza-cytosine, cisplatin, carboplatin, actinomycin D, asparaginase, bis-chloro-ethyl-nitroso-urea, bleomycin, chlorambucil, cyclohexyl-chloro-ethyl-nitroso-urea, cytosine arabinoside, daunomycin, etoposide, hydroxyurea, melphalan, mercaptopurine, mitomycin C, nitrogen mustard, procarbazine, teniposide, thioguanine, thiotepa, vincristine, 5-fluorouracil, 5-fluorocytosine, adriamycin, cyclophosphamide, methotrexate, vinblastine, doxorubicin (and liposomal doxorubicin), leucovorin, taxol, anti-estrogen agents such as tamoxifen, intracellular antibodies against oncogenes, the flavonol quercetin, Guan-mu-tong extract, retinoids, nontoxic retinoid analogs, sclerosants, immunotherapeutics, HERCEPTIN™, and monoterpenes.

13. The method of claim 3, wherein the material forms a map of the milk duct.

14. The method of claim 13, further comprising surgically removing at least a portion of the mapped milk duct.
